(19) United States
(12) Patent Application Publication
(10) Pub. No.: US 2004/0136998 A1
(43) Pub. Date: Jul. 15, 2004

(54) Methods and compositions for treating or preventing insulin-related disorders using binding agents specific for prostate specific membrane antigen

(76) Inventor: Neil H. Bander, New York, NY (US)

Correspondence Address: FISH & RICHARDSON PC, 225 FRANKLIN ST, BOSTON, MA, 02110, US

(21) Appl. No.: 10/688,015

(22) Filed: Oct. 17, 2003

Related U.S. Application Data
(60) Provisional application No. 60/422,396, filed on Oct. 30, 2002.

(51) Int. Cl. A61K 39/395

(52) U.S. Cl. 424/155.1

(57) ABSTRACT

Methods and compositions for treating, preventing, or diagnosing insulin-related disorders, e.g., diabetes mellitus, e.g., Type 2 diabetes, are disclosed. The methods and compositions of the invention use binding agents, e.g., antibodies, specific for the extracellular domain of human prostate specific membrane antigen (PSMA).

### CLAIM OF PRIORITY

This application claims priority under 35 USC § 119(e) to U.S. Patent Application Serial No. 60/422,396, filed on Oct. 30, 2002, the entire contents of which are hereby incorporated by reference.

### FIELD OF THE INVENTION

The invention relates to the use of a binding agent specific for prostate specific membrane antigen (PSMA) to treat or prevent insulin-related disorders, e.g., diabetes.

### BACKGROUND OF THE INVENTION

Insulin-related disorders are a group of diseases and disorders that are related to metabolism and include obesity, hyperglycemia, hypoglycemia, hyperinsulinemia, insulin-resistance and diabetes mellitus, as well as other disorders involving insulin-stimulated glucose transport.

Diabetes mellitus, one of the best-known insulin-related disorders, has reached epidemic proportions worldwide. Over the next decade the number of afflicted individuals is projected to exceed 200 million, possibly reaching 250 million persons. Diabetes mellitus is a chronic disease caused by an inherited and/or acquired deficiency in the production of insulin by the pancreas, or by the ineffectiveness of the insulin produced. This deficiency results in increased concentrations of glucose in the blood, which in turn damage many of the body's systems, in particular the blood vessels and nerves.

There are two principle variants of diabetes, as well as several other less common variants. Type 1 diabetes (also known as “insulin-dependent” diabetes) in which the pancreas fails to produce the insulin, which is essential for survival, develops most frequently in children and adolescents, but is being increasingly noted later in life. Type 2 diabetes (also known as “non-insulin-dependent” diabetes) results from the body's inability to respond properly to the action of insulin produced by the pancreas. Type 2 diabetes accounts for about 90% of all diabetes cases worldwide, and occurs most frequently in adults, but is increasingly being noted in adolescents as well. According to the WHO, Fact Sheet N^{o} 138, 4/02, approximately 150 million people have diabetes mellitus worldwide, and this number may well double by the year 2025.

Impaired glucose tolerance (IGT) and impaired fasting glycemia (IFG) refer to levels of blood glucose concentration above the normal range, but below those which are diagnostic for diabetes. Subjects with IGT and/or IFG are at substantially higher risk of developing diabetes and cardiovascular disease than those with normal glucose tolerance.

The health consequences of insulin-related disorders can be extremely severe. For instance, diabetes mellitus is associated with damage to the small blood vessels in the retina which results in loss of vision known as diabetic retinopathy, a leading cause of blindness and visual disability. After 15 years of living with diabetes, approximately 2% of people become blind, and about 10% develop severe visual impairment. Certain types of glaucoma and cataract may also be more common in people with diabetes.

Diabetes is also a leading cause of secondary kidney failure, but its frequency varies between populations and is also related to the severity and duration of the primary disease. Heart disease contributes to approximately 50% of all deaths among people with diabetes in industrialized countries. Risk factors for heart disease in people with diabetes include smoking, high blood pressure, high serum cholesterol and obesity.

Diabetic neuropathy, which can lead to sensory loss and damage to the limbs, as well as impotence in diabetic men, may be the most common complication of diabetes, as up to 50% of people with diabetes are affected to some degree. Major risk factors of diabetic neuropathy are the level and duration of elevated blood glucose.

Diabetic foot disease is one of the most costly complications of diabetes, especially in communities with inadequate footwear. Vascular and neurological disease processes associated with diabetic foot disease often lead to ulceration and subsequent limb amputation. Diabetes is the most common cause of non-traumatic amputation of the lower limb, which may be prevented by regular inspection and good care of the foot.

Diabetic ketoacidosis (DKA) and hyperglycemia hyperosmolar nonketotic coma (HHNC) are two of the most serious acute metabolic complications of diabetes. Although DKA most often occurs in patients with Type 1 diabetes mellitus, patients with Type 2 diabetes mellitus are also susceptible to DKA under certain conditions. DKA is characterized by high blood glucose, high ketone bodies, and metabolic acidosis, and occurs in 2-5% of people with Type 1 diabetes. The altered carbohydrate, lipid, and protein metabolism in DKA results in fluid and electrolyte deficiencies, impaired renal function, and metabolic acidosis. HHNC is associated with acute or subacute deterioration of CNS function and severe dehydration, low blood pressure, tachycardia, low grade fever, nausea, vomiting, distention, and pain, lethargy, hallucinations, and psychosis, seizures, hemiparesis, myoclonus, nystagmus, and rarely coma.

Existing methods for treating insulin-related disorders are generally sub-optimal. For example, conventional methods of treatment for diabetic and other patients who need exogenous insulin generally require that the patient undergo injection of insulin one or more times each day. Many people find it extremely difficult to comply with the diet and exercise requirements that are prescribed for the less severe forms of insulin-related disorders, leading to an increase in the severity of their condition and occurrence of the consequences mentioned above. In addition, a large percentage of the population is allergic to and therefore unable to take advantage of popular pharmaceutical treatments based on sulfonylurea. Absent strict adherence to the prescribed regimen of oral therapeutics or insulin and/or diet and exercise, diabetes patients experience a wide range of debilitating symptoms as described above, which can progress to coma and ultimately death.

Accordingly, there exists a need for improved therapeutic modalities for preventing and treating insulin-related disorders.

### SUMMARY OF THE INVENTION

The invention is based, in part, on the discovery that administration to a subject of an antibody to the extracellular domain of human prostate specific membrane antigen (PSMA) reduced the need for exogenous insulin in a patient suffering from diabetes. Following treatment with the anti-PSMA antibody, the patient, who was previously dependent on exogenous insulin for survival, was able to stop taking exogenous insulin for an extended period of time. Accordingly, the invention provides methods and compositions for treating or preventing insulin-related disorders using binding agents, e.g., antibodies or antigen binding fragments thereof, specific for PSMA, e.g., the extracellular region of PSMA. Methods of the invention can be used, for example, to treat or prevent a disease, e.g., an insulin-related disorder, by administering to the subject a PSMA binding agent in an amount effective to treat or prevent such disorder.

Examples of insulin-related disorders that can be treated or prevented using the methods of the invention include obesity, hyperglycemia, hypoglycemia, hyperinsulinemia, insulin-resistance, impaired glucose tolerance (IGT), impaired fasting glucose (IFG) and diabetes mellitus (e.g. Type 1 or Type 2 diabetes) as well as other disorders of glycemia, including those due to genetic defects of β cell function; genetic defects in insulin action; diseases of the exocrine pancreas; endocrinopathies; drug or chemical induced disorders of glycemia; infections; uncommon forms of immune-mediated diabetes; other genetic syndromes sometimes associated with diabetes; or gestational diabetes. Typically, the insulin-related disorder is Type II diabetes.

In some embodiments, the methods and compositions can be used to treat or prevent an insulin-related disorder in a subject having a co-existing, aggravating or precipitating condition such as a condition characterized by unwanted cell proliferation, e.g., cancer or non-malignant, hyperproliferative disorders, e.g., a skin disorder, e.g., psoriasis, or other disorders associated with the expression of PSMA in the neovasculature. Cancerous disorders include prostate cancer. Non-malignant conditions include benign hyperproliferation of the prostate. Preferably, administration of a binding agent specific for PSMA can alleviate at least one symptom of the insulin-related disorder as well as at least one symptom of the co-existing, aggravating, or precipitating condition.

The present method can be performed, e.g., on cells present in a subject, e.g., a human or animal subject, e.g., an experimental animal subject, as part of an in vivo (e.g., therapeutic or prophylactic) protocol.

In preferred embodiments, the binding agent used in the methods and compositions of the invention, interacts with, e.g., binds to, to PSMA, preferably human PSMA, with high affinity and specificity. For example, the binding agent binds to human PSMA with an affinity constant of at least 10⁷ M⁻¹, preferably, between 10⁸ M⁻¹ and 10¹⁰ M⁻¹, or about 10⁹ M⁻¹. Preferably, the binding agent binds to the extracellular domain of PSMA, and most preferably, the extracellular domain of human PSMA (e.g., amino acids 44-750 of human PSMA).

The binding agent can be an antibody (e.g., a monospecific, or a recombinant or modified antibody) or an antigen-binding fragment thereof, a small molecule, or a PSMA ligand. Preferably, the modified antibodies are those having one or more complementarity determining regions (CDRs) from a J591, J415, J533 or E99 antibody.

In a preferred embodiment, the binding agent is an anti-PSMA monospecific antibody (e.g., a monoclonal antibody) or an antigen-binding fragment thereof. The anti-PSMA antibodies (e.g., recombinant or modified antibodies) can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)₂ or scFv fragment, or one or more CDRs). An antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-PSMA antibody includes a heavy and light chain constant region substantially from a human antibody, e.g., a human IgG1 constant region or a portion thereof. In some embodiments, the anti-PSMA antibodies are human antibodies.

The antibody (or fragment thereof) can be a murine or a human antibody. Examples of preferred murine monoclonal antibodies that can be used include a E99, J415, J533 and J591 antibody, which are produced by hybridoma cell lines having an ATCC Accession Number HB-12101, HB-12109, HB-12127, and HB-12126, respectively. Also within the scope of the invention are methods and composition using antibodies, or antigen-binding fragments thereof, which bind overlapping epitopes of, or competitively inhibit, the binding of the anti-PSMA antibodies disclosed herein to PSMA, e.g., antibodies which bind overlapping epitopes of, or competitively inhibit, the binding of monoclonal antibodies E99, J415, J533 or J591 to PSMA. Any combination of anti-PSMA antibodies can be used, e.g., two or more antibodies that bind to different regions of PSMA, e.g., antibodies that bind to two different epitopes on the extracellular domain of PSMA.

In some embodiments, the binding agent is an anti-PSMA antibody that binds to all or part of the epitope of an antibody described herein, e.g., a J591, E99, J415, and J533 antibody. The anti-PSMA antibody can inhibit, e.g., competitively inhibit, the binding of an antibody described herein, e.g., a J591, E99, J415, and J533 antibody, to human PSMA. An anti-PSMA antibody may bind to an epitope, e.g., a conformational or a linear epitope, which epitope when bound prevents binding of an antibody described herein, a J591, E99, J415, and J533 antibody. The epitope can be in close proximity spatially or functionally associated, e.g., an overlapping or adjacent epitope in linear sequence or conformationally to the one recognized by the J591, E99, J415, or J533 antibody.

In one embodiment, the anti-PSMA antibody binds to an epitope located wholly or partially within the region of about amino acids 120 to 500, preferably 130 to 450, more preferably, 134 to 437, or 153 to 347, of human PSMA. Preferably, the epitope includes at least one glycosylation site, e.g., at least one N-linked glycosylation site (e.g., the N-linked glycosylation site located at about amino acids 190-200, preferably at about amino acid 195, of human PSMA).

In other embodiments, the antibodies (or fragments thereof) are a recombinant or modified anti-PSMA antibody chosen from, e.g., a chimeric, a humanized, a deimmunized, or an in vitro generated antibody. As discussed herein, the modified antibodies can be CDR-grafted, humanized, deimmunized, or more generally, antibodies having CDRs from a non-human antibody, e.g., murine J591, J415, J533 or E99 antibody and a framework that is selected as less immunogenic in humans, e.g., less antigenic than the murine framework in which a murine CDR naturally occurs. In one embodiment, a modified antibody is a deimmunized anti-PSMA antibody, e.g., a deimmunized form of E99, J415, J533 or J591 (e.g., a deimmunized form of an antibody produced by a hybridoma cell line having an ATCC Accession Number HB-12101, HB-12109, HB-12127 and HB-12126, respectively). Preferably, the antibody is a deimmunized form of J591 or J415 (referred to herein as “deJ591” or “deJ415” respectively). Most preferably, the antibody is a deimmunized form of J591.

The binding agent, e.g., the anti-PSMA antibody, or antigen-binding fragment thereof, described herein can be used alone, e.g., can be administered to a subject, or used in vitro, in non-derivatized or unconjugated forms.

In some embodiments, the binding agent, e.g., an anti-PSMA antibody or fragment thereof, is also internalized with PSMA which permits intercellular delivery of a molecular entity conjugated to the antibody. The binding agent, e.g., an anti-PSMA antibody, or antigen-binding fragment thereof, can be derivatized or linked to another molecular entity, typically a label or a therapeutic (e.g., a cytotoxic or cytostatic) agent. The molecular entity can be, e.g., another peptide, protein, a non-peptide chemical compound, isotope, etc. The anti-PSMA antibody, or antigen-binding fragment thereof, can be functionally linked, e.g., by chemical coupling, genetic fusion, non-covalent association or otherwise, to one or more other molecular entities. For example, the anti-PSMA antibody, or antigen-binding fragment thereof, can be coupled to a label, such as a fluorescent label, a biologically active enzyme label, a radioisotope (e.g., a radioactive ion), a nuclear magnetic resonance active label, a luminescent label, or a chromophore. In other embodiments, the anti-PSMA antibody, or antigen-binding fragment thereof, can be coupled to a therapeutic agent, e.g., a cytotoxic moiety, e.g., a therapeutic drug, a radioisotope, molecules of plant, fungal, or bacterial origin, or biological proteins (e.g., protein toxins), or mixtures thereof. The therapeutic agent can be an intracellularly active drug or other agent, such as short-range radiation emitters, including, for example, short-range, high-energy α-emitters, as described herein. In some preferred embodiments, the anti-PSMA antibody, or antigen binding fragment thereof, can be coupled to a molecule of bacterial origin, e.g., a maytansinoid (e.g., maytansinol or the DM1 maytansinoid). A radioisotope can be an α-, β-, or γ-emitter, or an β- and γ-emitter. Radioisotopes useful as therapeutic agents include yttrium (⁹⁰Y), lutetium (¹⁷⁷Lu), actinium (²²⁵ Ac), praseodymium, astatine (²¹¹At), rhenium (¹⁸⁶Re), bismuth (²¹²Bi or ²¹³ Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one of the therapeutic isotopes listed above. The anti-PSMA antibody, or antigen-binding fragment thereof can also be linked to another antibody to form, e.g., a bispecific or a multispecific antibody.

The subject can be a mammal, e.g., a primate, preferably a higher primate, e.g., a human (e.g., a patient having, or at risk of, a disorder described herein, e.g., an insulin-related disorder). In one embodiment, the subject is a patient having diabetes mellitus, e.g., Type 1 or Type 2 diabetes, preferably, the subject has Type 2 diabetes. The subject can also be an experimental animal, e.g., an animal model for an insulin-related disorder, e.g., a NOD mouse, an ob/ob mouse, a db/db mouse, a Zucker fatty rat, or a streptozotocin-induced mouse or rat. In some embodiments, the subject further has a co-existing, aggravating or precipitating condition, e.g., a condition which can directly or indirectly exacerbate the symptoms of the insulin-related disorder. In one embodiment, the co-existing, aggravating or precipitating condition can be a disorder characterized by unwanted cell proliferation, e.g., cancer (e.g., prostate or non-prostate cancer), or other non-malignant hyperproliferative disorders. In other embodiments, the co-existing, aggravating or precipitating condition is a skin disorder, e.g., psoriasis. Thus, in some embodiments, the subject can be a patient having both an insulin-related disorder, e.g., diabetes mellitus, e.g., Type 2 diabetes, and a disorder characterized by unwanted cell proliferation, e.g., cancer, or a skin disorder, e.g., psoriasis.

The PSMA binding agent, e.g., a PSMA binding agent as described herein, can be administered to the subject systemically (e.g., intravenously, intramuscularly, by infusion, e.g., using an infusion device, subcutaneously, transdermally, or by inhalation). In those embodiments where the PSMA binding agent is a small molecule, it can be administered orally.

The methods of the invention can further include the step of monitoring the subject, e.g., for an improvement in one or more indicators of diabetes, e.g., reduced insulin dependence, increased sensation in response to monofilament stimulation, increased C-peptide levels, increased or decreased urine glucose levels, decreased urine ketone levels, reduced glycated hemoglobin (Hba1c) levels, reduced fasting plasma glucose levels, increased or decreased SMBG results, reduced lipid levels, reduced microalbumin levels/negative microalbuminuria tests, reduced BUN (Blood Urea Nitrogen) levels, and/or decreased serum creatinine levels; a reduction in the subject's symptoms, e.g., diabetic neuropathy, diabetic foot disease, renal impairment, heart disease, and/or diabetic retinopathy, or any parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same PSMA binding agent or for additional treatment with additional agents. Generally, a decrease in one or more of the parameters described above is indicative of the improved condition of the subject.

The methods of the invention can further include the step of analyzing a nucleic acid or protein from the subject, e.g., analyzing the genotype of the subject. In one embodiment, a nucleic acid encoding human PSMA and/or an upstream or downstream component(s) of human PSMA signaling, e.g., an extracellular or intracellular activator or inhibitor of human PSMA, is analyzed. The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the PSMA binding agent to thereby determine appropriate dosage(s) and treatment regimen(s) of the PSMA binding agent (e.g., amount per treatment or frequency of treatments) for prophylactic or therapeutic treatment of the subject.

The invention also features methods and compositions that can be used in combination with other therapeutic modalities. Accordingly, the PSMA binding agent can be administered in combination with a systemic agent selected from the group consisting of systemic glucocorticoids, sulfones, aminoquinolines, cytotoxic agents, antimetabolic agents, retinoids, antihistamines, immunosuppressive drugs, immunomodulatory drugs, thalidomide, and agents useful in the treatment of diabetes, e.g., insulin, sulfonylureas (e.g., meglitinides and nateglinides), biguanides, thiazolidinediones, and alpha-glucosidase inhibitors. The PSMA binding agent and the systemic agent may be administered sequentially or simultaneously.

Any suitable combination and sequence of systemic agents can be used. The PSMA binding agent and the systemic agents can be administered during periods of active disease (e.g., when the subject is actively suffering from one or both of an insulin-related disorder and a co-existing, aggravating or precipitating condition such as a condition characterized by unwanted cell proliferation), and/or during a period of remission or less active disease (e.g., when one or both of an insulin-related disorder and a co-existing, aggravating or precipitating condition are in remission or reduced in severity). The PSMA binding agent and the systemic agents can be administered before treatment, concurrently with treatment, post treatment, or during remission of the disorder.

The invention also features a composition for systemic administration, which includes a binding agent which binds specifically to PSMA, e.g., a binding agent as described herein, and an agent (also referred to herein as a “second agent”) that reduces, ameliorates, or prevents an insulin-related disorder. In other embodiments, the invention features methods and compositions that can be used in combination with an agent that reduces, ameliorates, or prevents an insulin-related disorder. Examples of agents that can be used in the compositions of the invention in combination with the binding agent include, but are not limited to agents useful in the treatment of diabetes, e.g., insulin, sulfonylureas (e.g., meglitinides and nateglinides), biguanides, thiazolidinediones, and alpha-glucosidase inhibitors. The PSMA binding agent and the secondary agent can be administered sequentially or simultaneously. In yet other embodiments, the methods can be used in combination with immunomodulatory agents, e.g., interleukin-2 (IL-2) or interferon (IFN).

In preferred embodiments, the amount of a second agent therapeutically effective to treat a subject having an insulin-related disorder is reduced compared to the amount of the second agent therapeutically effective to treat the subject in the absence of the PSMA binding agent. For example, in one embodiment, the subject has diabetes and the amount of exogenous insulin administered to treat the diabetes is reduced or eliminated as compared to the amount of exogenous insulin administered to treat diabetes in the subject in the absence of the PSMA binding agent. The reduction and/or elimination of a second agent in a subject having an insulin-related disorder can be over an extended period of time, e.g., 15 days, 1 month, 2 months, 3 months, 6 months, 1, 2, 3, 4 years or more. In another embodiment, the amount of the second agent administered to treat the insulin-related disorder is reduced by at least 5%, 10%, 20%, 30%, 50% or more as compared to the amount administered in the absence of administeration of the PSMA binding agent. The reduction of the second agent can refer to the time frame of the administration(s) or the amount of the administration or both.

In other embodiments, the methods of the invention include administering to the subject a PSMA binding agent, e.g., a PSMA binding agent as described herein, in combination with a cytotoxic agent, in an amount effective to treat or prevent an insulin-related disorder. The binding agent and the cytotoxic agent can be administered simultaneously or sequentially. In one embodiment, the combination of the PSMA binding agent and cytotoxic agent may treat, e.g., kill or ablate a cell, e.g., a PSMA-expressing cell, e.g., a prostate cell, a vascular endothelial cell proximate to the cell, or an epidermal or dermal cell, associated with a co-existing, aggravating or precipitating condition, and thereby treat or prevent an insulin-related disorder, e.g., an insulin-related disorder exacerbated by the co-existing or precipitating condition.

Exemplary cytotoxic agents that can be administered in combination with the PSMA binding agents include antimicrotubule agents, topoisomerase inhibitors, antimetabolites, mitotic inhibitors, alkylating agents, intercalating agents, agents capable of interfering with a signal transduction pathway, agents that promote apoptosis and radiation, cyclophosphamide, busulfan, dibromomannitol, streptozotocin, mitomycin C, cis-dichlorodiamine platinum (II) (DDP) cisplatin), anthracyclines, and anti-mitotic agents, inter alia.

In preferred embodiments, the invention features methods and compositions for treating a subject having an insulin-related disorder as well as unwanted cell proliferation, e.g., cancer, or a skin disease, e.g., psoriasis, by administering to the subject a PSMA binding agent in combination with one or more systemic agents and/or cytotoxic agents. The systemic agent may be as described herein, e.g., agents useful in the treatment of insulin-related disorders, e.g., diabetes. The cytotoxic agent may be as described herein, e.g., an agent useful in the treatment of a disorder characterized by unwanted cell proliferation, e.g., cancer, or a skin disease, e.g., psoriasis.

The compositions of the invention can further include a pharmaceutically acceptable carrier, excipient or stabilizer.

In another aspect, the invention features methods of diagnosing or screening for subjects at risk for an insulin-related disorder, e.g., diabetes, by screening for elevated or otherwise abnormal PSMA levels to evaluate the subject for a disorder (e.g., a disorder characterized by unwanted cell proliferation, e.g., cancer, or a skin disorder, e.g., psoriasis) which can directly or indirectly exacerbate an insulin related disorder, to evaluate if the subject is at risk for developing an insulin-related disorder.

Subjects at risk for insulin-related disorders such as diabetes include obese subjects, subjects with impaired glucose tolerance, and subjects having hyperinsulinemia.

A subject who has been determined to be at risk for an insulin-related disorder can then be treated for the disorder, e.g., by administration of a PSMA binding agent, but prophylactic measures can be used to prevent the insulin-related disorder, e.g., by modification in diet or exercise, by administration of oral anti-diabetics, or other methods known in the art.

In another aspect, the invention features a kit, which includes a PSMA binding agent, e.g., a PSMA binding agent as described herein, for use alone or in combination with a systemic agent, e.g., a second agent as described herein, along with instructions on how to use the PSMA binding agent or the combination of such agents, e.g., to treat or prevent an insulin-related disorder or an insulin-related disorder in conjunction with a co-existing, aggravating, or precipitating condition, e.g., cancer, e.g., prostate or non-prostate cancer.

Other features and advantages of the instant invention will become more apparent from the following detailed description and claims.

### BRIEF DESCRIPTION OF THE DRAWINGS

FIGS. 1A-1B depict the amino acid sequence of murine J591 heavy and light chain variable region, respectively. The location of the CDRs is indicated in the Figures; the amino acid numbering is according the Kabat numbering (see, Kabat, E. A., et al. (1991) Sequences of Proteins of Immunological Interest, Fifth Edition, U.S. Department of Health and Human Services, NIH Publication No. 91-3242). Note that the CDRs are considered to encompass the Chothia loops and the Kabat hypervariable regions together and the sequences have been annotated accordingly. Heavy Chain: CDR1 is depicted in SEQ ID NO:1; CDR2 is depicted in SEQ ID NO:2; CDR3 is depicted in SEQ ID NO:3; the framework excluding CDR regions is depicted in SEQ ID NO:7; and the framework including CDR regions is depicted in SEQ ID NO:19. Light Chain: CDR1 is depicted in SEQ ID NO:4; CDR2 is depicted in SEQ ID NO:5; CDR3 is depicted in SEQ ID NO:6; the framework excluding CDR regions is depicted in SEQ ID NO:8; and the framework including CDR regions is depicted in SEQ ID NO:20.

FIGS. 2A-2B depict the amino acid sequence of deimmunized J591 heavy and light chain variable region, respectively. The location of the CDRs is indicated in the Figures; the amino acid numbering is according the Kabat numbering (see, Kabat, E. A., et al. (1991) supra). Note that the CDRs are considered to encompass the Chothia loops and the Kabat hypervariable regions together and the sequences have been annotated accordingly. Heavy Chain: CDR1 is depicted in SEQ ID NO:1; CDR2 is depicted in SEQ ID NO:2; CDR3 is depicted in SEQ ID NO:3; framework 1 is depicted in SEQ ID NO:9; framework 2 is depicted in SEQ ID NO:10; framework 3 is depicted in SEQ ID NO:11; framework 4 is depicted in SEQ ID NO:12; the framework excluding CDR regions is depicted in SEQ ID NO:17; and the framework including CDR regions is depicted in SEQ ID NO:21. Light Chain: CDR1 is depicted in SEQ ID NO:4; CDR2 is depicted in SEQ ID NO:5; CDR3 is depicted in SEQ ID NO:6; framework 1 is depicted in SEQ ID NO:13; framework 2 is depicted in SEQ ID NO:14; framework 3 is depicted in SEQ ID NO:15; framework 4 is depicted in SEQ ID NO:16; the framework excluding CDR regions is depicted in SEQ ID NO:18; and the framework including CDR regions is depicted in SEQ ID NO:22.

FIGS. 3A-3B depict an alignment of the murine J591 and deimmunized heavy chain variable regions, respectively (3A; SEQ ID NO:19 and 21, respectively) and light chain variable regions (3B; SEQ ID NO:20 and 22, respectively). Potential T cell epitopes (identified using a peptide threading program) in murine J591 VH and VK are shown in FIGS. 3A-3B, respectively. The 13-mer peptides predicted to bind to MHC class II are marked by the underlining, the CDRs are located at residues 26 to 35, 50 to 66 and 99 to 104 of FIG. 3A, and residues 24 to 34, 50 to 56 and 89 to 97 of FIG. 3B, and residues altered in the deimmunized heavy and light chain variable regions are boxed. Where possible, amino acid substitutions are those commonly used in human germline VH regions. The amino acid numbering is linear, not according to Kabat.

FIGS. 4A-4B depict the nucleotide sequences of the deimmunized J591 heavy and light chain variable region, respectively. FIG. 4A shows an alignment of the coding and noncoding nucleotide strands of deimmunized J591 heavy chain variable region (SEQ ID NOs:23 and 24, respectively) with the corresponding amino acid sequence (SEQ ID NO:27). FIG. 4B shows an alignment of the coding and noncoding nucleotide strands of deimmunized J591 light chain variable region (SEQ ID NOs:25 and 26, respectively) with the corresponding amino acid sequence (SEQ ID NO:28). The location of the signal peptide and CDRs 1-3 is indicated in each alignment.

FIG. 5 depicts an alignment of the amino acid sequences for the murine and several deimmunized heavy chain variable regions of the J415 antibody. The murine amino acid sequence is shown as J415VH (SEQ ID NO:47); the deimmunized sequences are depicted as J415DIVH1 (amino acid residues 18 to 133 of SEQ ID NO:54), J415DIVH2 (SEQ ID NO:59), J415DIVH3 (SEQ ID NO:60), and J415DIVH4 (SEQ ID NO:49). The preferred sequence is J415DIVH4 (SEQ ID NO:49). The amino acid replacements are indicated by the boxed residues. A consensus sequence is labeled “majority” (SEQ ID NO:61).

FIG. 6 depicts an alignment of the amino acid sequences for the murine and several deimmunized light chain variable regions of the J415 antibody. The murine amino acid sequence is shown as J415VK (SEQ ID NO:48); the deimmunized sequences are depicted as J415DIVK1 (amino acid residues 18 to 124 of SEQ ID NO:57), J41-5DIVK2 (SEQ ID NO:62), J415DIVK3 (SEQ ID NO:63), J415DIVK4 (SEQ ID NO:64), J415DIVK5 (SEQ ID NO:50), J415DIVK6 (SEQ ID NO:65), J415DIVK7 (SEQ ID NO:66), and J415DIVK8 (SEQ ID NO:67). The preferred sequence is J415DIVK5 (SEQ ID NO:50). The amino acid replacements are indicated by the boxed residues. A consensus sequence is labeled “majority” (SEQ ID NO:68).

FIG. 7A depicts the nucleic acid coding sequence, the amino acid sequence, and the nucleic acid reverse complement sequence of the deimmunized J415 heavy chain variable region (J415DIVH1) (SEQ ID NO:53-55, respectively). The relative location of the signal sequence, intron and J415DIVH 1 amino acid sequence is indicated, as well as some restriction sites.

FIG. 7B depicts the nucleic acid coding sequence, the amino acid sequence, and the nucleic acid reverse complement sequence of the murine J415 heavy chain variable region (SEQ ID NO:125, 47, and 126, respectively). The relative locations of the CDRs and some restriction sites are indicated.

FIG. 7C depicts an alignment of the amino acid sequence of the murine J415 heavy chain variable region (SEQ ID NO:47) and a consensus sequence for Kabat subgroup murine VHIIIC (MUVHIII, SEQ ID NO:69). A consensus majority sequence based on the alignment is also shown (SEQ ID NO:70).

FIG. 8A depicts the nucleic acid coding sequence, the amino acid sequence, and the nucleic acid reverse complement sequence of the deimmunized J415 light chain variable region (J415DIVK1) (SEQ ID NO:56-58, respectively). The relative location of the signal sequence, intron and J415DIVK1 amino acid sequence is indicated, as well as some restriction sites.

FIG. 8B depicts the nucleic acid coding sequence, the amino acid sequence, and the nucleic acid reverse complement sequence of the murine J415 light chain variable region (SEQ ID NOs:127, 48, and 128, respectively). The relative locations of the CDRs and some restriction sites are also indicated.

FIG. 8C depicts an alignment of the amino acid sequence of the murine J415 light chain variable region (SEQ ID NO:48) and a consensus sequence for Kabat subgroup murine variable light chain (MuVKI, SEQ ID NO:71). A consensus majority sequence based on the alignment is also shown (SEQ ID NO:72).

FIG. 9A depicts the nucleic acid coding sequence, the amino acid sequence, and the nucleic acid reverse complement sequence of the murine J533 heavy chain variable region (SEQ ID NO:73-75, respectively). The relative locations of the CDRs and restriction sites are indicated.

FIG. 9B depicts an alignment of the amino acid sequence of the murine J533 heavy chain variable region (SEQ ID NO:74) and a consensus sequence for Kabat subgroup murine variable heavy chain (MuVHIIA, SEQ ID NO:79). A consensus majority sequence based upon the alignment is also shown (SEQ ID NO:80).

FIG. 10A depicts the nucleic acid coding sequence, the amino acid sequence, and the nucleic acid reverse complement sequence of the murine J533 light chain variable region (SEQ ID NO:76-78, respectively). The relative locations of the CDRs and some restriction sites are indicated.

FIG. 10B depicts an alignment of the amino acid sequence of the murine J533 light chain variable region (SEQ ID NO:77) and a consensus sequence for Kabat subgroup murine MuVKIII, SEQ ID NO:81). A consensus majority sequence based upon the alignment is also shown (SEQ ID NO:82).

FIG. 11A depicts the nucleic acid coding sequence, the amino acid sequence, and the nucleic acid reverse complement sequence of the murine E99 heavy chain variable region (SEQ ID NO:83-85, respectively). The relative locations of the CDRs and some restriction sites are indicated.

FIG. 11B depicts an alignment of the amino acid sequence of the murine E99 heavy chain variable region (SEQ ID NO:84) and a consensus sequence for Kabat subgroup murine variable heavy chain (MuVHIB, SEQ ID NO:89). A consensus majority sequence based upon the alignment is also shown (SEQ ID NO:90).

FIG. 12A depicts the nucleic acid coding sequence, the amino acid sequence, and the nucleic acid reverse complement sequence of the murine E99 light chain variable region (SEQ ID NO:86-88, respectively). The relative locations of the CDRs and some restriction sites are indicated.

FIG. 12B depicts an alignment of the amino acid sequence of the murine E99 light chain variable region-(SEQ ID NO:87) and a consensus sequence for Kabat subgroup murine variable light chain (MuVKI, SEQ ID NO:91). A consensus majority sequence based upon the alignment is also shown (SEQ ID NO:92).

### DETAILED DESCRIPTION OF THE INVENTION

The invention is based, in part, on the discovery that administration of an antibody to the extracellular domain of prostate specific membrane antigen (PSMA) reduced the need for exogenous insulin in a patient suffering from prostate cancer and an insulin-related disorder, namely, diabetes. The patient was initially treated with the anti-PSMA antibody to treat his prostate cancer. Following treatment with the anti-PSMA antibody, the patient, who was previously dependent on exogenous insulin for survival, was able to completely stop taking exogenous insulin for an extended period of time. Methods of using the PSMA binding agents to treat insulin-related disorders as well as treat, e.g., ablate or kill, PSMA-expressing cells, are encompassed by the invention. Accordingly, in one aspect, the invention provides methods and compositions for treating or preventing insulin-related disorders using binding agents, e.g., antibodies or antigen-binding fragments thereof, specific for PSMA. In some embodiments, the invention further provides methods and compositions for treating or preventing insulin-related disorders secondary to or associated with a primary disorder characterized by unwanted cellular proliferation. As used herein “secondary to” refers to a disorder that is precipitated, e.g., brought on, or aggravated, e.g., made worse, by another disorder, e.g., a primary disorder. The term “exacerbated by” is also used to refer to insulin-related disorders which are secondary to another disorder or condition. For example, an insulin-related disorder, e.g., diabetes mellitus, can be exacerbated by or secondary to, e.g., precipitated or aggravated by, a primary disorder characterized by unwanted cell proliferation, e.g., cancer, e.g., prostate or non-prostate cancer.

In order that the present invention may be more readily understood, certain terms are first defined. Additional definitions are set forth throughout the detailed description.

As used herein, “PSMA” or “prostate-specific membrane antigen” protein refers to mammalian PSMA, preferably human PSMA protein. Human PSMA includes the two protein products, PSMA and PSM′, encoded by the two alternatively spliced mRNA variants (containing about 2,653 and 2,387 nucleotides, respectively) of the PSMA cDNA disclosed in Israeli et al. (1993) Cancer Res. 53:227-230; Suet al. (1995) Cancer Res. 55:1441-1443; U.S. Pat. No. 5,538,866, U.S. Pat. No. 5,935,818, and WO 97/35616, the contents of which are hereby incorporated by reference. The long transcript of PSMA encodes a protein product of about 100-120 kDa molecular weight characterized as a type II transmembrane receptor having sequence identity with the transferrin receptor and having NAALADase activity (Carter et al. (1996) Proc. Natl. Acad. Sci. USA 93:749-753). Accordingly, the term “human PSMA” refers to at least two protein products, human PSMA and PSM′, which have or are homologous to (e.g., at least about 85%, 90%, 95% identical to) an amino acid sequence as shown in Israeli et al. (1993) Cancer Res. 53:227-230; Su et al. (1995) Cancer Res. 55:1441-1443; U.S. Pat. No. 5,538,866, U.S. Pat. No. 5,935,818, and WO 97/35616; or which is encoded by (a) a naturally occurring human PSMA nucleic acid sequence (e.g., Israeli et al. (1993) Cancer Res. 53:227-230 or U.S. Pat. No. 5,538,866); (b) a nucleic acid sequence degenerate to a naturally occurring human PSMA sequence; (c) a nucleic acid sequence homologous to (e.g., at least about 85%, 90%, 95% identical to) the naturally occurring human PSMA nucleic acid sequence; or (d) a nucleic acid sequence that hybridizes to one of the foregoing nucleic acid sequences under stringent conditions, e.g., highly stringent conditions.

A “PSMA binding agent” is an agent which interacts with (e.g., binds to) PSMA, preferably human PSMA. Preferably, the PSMA binding agent interact with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866). Preferably, the interaction, e.g., binding, occurs with high affinity, e.g., affinity constant of at least 10⁷ M⁻¹, preferably, between 10⁸ M⁻¹ and 10¹⁰ M⁻¹, or about 10⁹ M⁻¹, and specificity. Preferably, the PSMA binding agent treats, e.g., ablates or kills, a cell, e.g., a PSMA-expressing cell. The mechanism by which the PSMA binding agent treats, e.g., ablates or kills, the cell is not critical to the practice of the invention. In one embodiment, the PSMA binding agent may bind to and be internalized with the PSMA expressed in the cells and/or vascular endothelial cells proximate to the cells. In those embodiments, the binding agent can be used to target a second moiety, e.g., a cytotoxic agent, to the cell. The cell can be killed directly by the PSMA binding agent by binding directly to the cell or the vascular endothelial cells proximate thereto. Alternatively, the PSMA binding agent can treat, e.g., kill or ablate, or otherwise change the properties of the vascular endothelial cells to which it binds so that blood flow to the cells proximate thereto is reduced, thereby causing the cells to be killed or ablated. Examples of PSMA binding agents include anti-PSMA antibodies (e.g., monospecific, monoclonal (e.g., human or rodent), recombinant or modified, e.g., chimeric, CDR-grafted, humanized, deimmunized, in vitro generated antibodies; small molecules or peptidomimetics.

An “anti-PSMA antibody” is an antibody that interacts with (e.g., binds to) PSMA, preferably human PSMA protein. Preferably, the anti-PSMA antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866). In one embodiment, the anti-PSMA antibody binds all or part of the epitope of an antibody described herein, e.g., J591, E99, J415, and J533. The anti-PSMA antibody can inhibit, e.g., competitively inhibit, the binding of an antibody described herein, e.g., J591, E99, J415, and J533, to human PSMA. An anti-PSMA antibody may bind to an epitope, e.g., a conformational or a linear epitope, which epitope when bound prevents binding of an antibody described herein, J591, E99, J415, and J533. The epitope can be in close proximity spatially or functionally-associated, e.g., an overlapping or adjacent epitope in linear sequence or conformationally to the one recognized by the J591, E99, J415, or J533 antibody. In one embodiment, the anti-PSMA antibody binds to an epitope located wholly or partially within the region of about amino acids 120 to 500, preferably 130 to 450, more preferably, 134 to 437, or 153 to 347, of human PSMA (amino acid residues correspond to the human PSMA sequence disclosed in U.S. Pat. No. 5,538,866). Preferably, the epitope includes at least one glycosylation site, e.g., at least one N-linked glycosylation site (e.g., the N-linked glycosylation site located at about amino acids 190-200, preferably at about amino acid 195, of human PSMA) (amino acid residues correspond to the human PSMA sequence disclosed in U.S. Pat. No. 5,538,866).

In a preferred embodiment, the interaction, e.g., binding, between an anti-PSMA antibody and PSMA occurs with high affinity (e.g., affinity constant of at least 107 M⁻¹, preferably, between 10⁸ M⁻¹ and 10¹⁰, or about 10⁹ M⁻¹) and specificity. Preferably, the anti-PSMA antibody treats, e.g., ablates or kills, a cell, e.g., a PSMA-expressing cell. The mechanism by which the anti-PSMA antibody treats, e.g., ablates or kills, the cell is not critical to the practice of the invention. In one embodiment, the anti-PSMA antibody may bind to and be internalized with the PSMA expressed in the cells and/or vascular endothelial cells proximate to the cells. In those embodiments, the anti-PSMA antibody can be used to target a second moiety, e.g., a cytotoxic or labeling agent, to the cell. In other embodiments, the anti-PSMA antibody may mediate host-mediated-killing, e.g., complement- or ADCC-mediated killing, of the cell and/or the vascular cell proximate thereto, upon binding to the extracellular domain of PSMA. The cell can be killed directly by the anti-PSMA antibody by binding directly to the cell or the vascular endothelial cells proximate thereto. Alternatively, the anti-PSMA antibody can treat, e.g., kill or ablate, or otherwise change the properties of the vascular endothelial cells to which it binds so that blood flow to the cells proximate thereto is reduced, thereby causing the cells to be killed or ablated. Examples of anti-PSMA antibodies include, e.g., monospecific, monoclonal (e.g., human), recombinant or modified, e.g., chimeric, CDR-grafted, humanized, deimmunized, and in vitro generated anti-PSMA antibodies.

As used herein, the term “treat” or “treatment” is defined as the application or administration of a PSMA binding agent to a subject, e.g., a patient, or application or administration to an isolated tissue or cell from a subject, e.g., a patient, which tissue or cell is returned to the patient. The binding agent can be administered alone, or in combination with a second agent. The subject can be a patient having an insulin-related disorder (e.g., an insulin-related disorder as described herein), a symptom of an insulin-related disorder or a predisposition toward an insulin-related disorder. The treatment can be to cure, heal, alleviate, relieve, alter, remedy, ameliorate, improve or affect the disorder, the symptoms of the disorder or the predisposition toward the disorder. While not wishing to be bound by theory, treating is believed to cause the inhibition, ablation, or killing of a cell in vitro or in vivo, or otherwise reducing capacity of a cell, e.g., an aberrant cell, or a pathway associated with a PSMA-expressing cell, to mediate a disorder, e.g., a disorder as described herein (e.g., an insulin-related disorder, e.g., diabetes mellitus, e.g., Type 2 diabetes). Further, treating is believed to cause the inhibition, ablation, or killing of a cell in vitro or in vivo, or otherwise reducing capacity of a cell, e.g., an aberrant cell, or a pathway associated with a PSMA-expressing cell, to mediate a primary disorder, e.g., a primary disorder associated with unwanted cellular proligeration, that precipitates or aggravates, or exacerbates, a secondary insulin-related disorder as described herein (e.g., diabetes mellitus, e.g., Type 2 diabetes).

As used herein, an amount of a PSMA binding agent, e.g., an anti-PSMA antibody, effective to treat a disorder, or a “therapeutically effective amount” refers to an amount of the agent which is effective, upon single or multiple dose administration to a subject, in treating a cell (e.g., a PSMA-expressing cell, or a vascular cell proximate thereto), or in prolonging curing, alleviating, relieving or improving a subject with the disorder as described herein, beyond that expected in the absence of such treatment.

As used herein, an amount of a PSMA binding agent, e.g., an anti-PSMA antibody, effective to prevent a disorder, e.g., an insulin-related disorder as described herein, or a “prophylactically effective amount” of the agent refers to an amount of a binding agent, e.g., an anti-PSMA antibody, e.g., an anti-PSMA antibody as described herein, which is effective, upon single- or multiple-dose administration to the subject, in preventing or delaying the occurrence of the onset or recurrence of the disorder, or treating a symptom thereof.

The terms “induce,” “inhibit,” “potentiate,” “elevate,” “increase,” “decrease,” or the like, e.g., which denote quantitative differences between two states, refer to a difference, e.g., a statistically significant difference, between the two states. For example, “an amount effective to inhibit the proliferation of the PSMA-expressing hyperproliferative cells” means that the rate of growth of the cells will be different, e.g., statistically significantly different, from the untreated cells.

In another example, “an amount of a second agent to treat an insulin-related disorder” means that the dosage of the second agent will be different, e.g., statistically different, than the dosage in the absence of administration of a PSMA binding agent.

As used herein, “specific binding” refers to the property of the binding agent, preferably the antibody, to: (1) to bind to PSMA, e.g., human PSMA protein, with an affinity of at least 1×10⁷ M⁻¹, and (2) preferentially bind to PSMA, e.g., human PSMA protein, with an affinity that is at least two-fold, 50-fold, 100-fold, 1000-fold, or more greater than its affinity for binding to a non-specific antigen (e.g., BSA, casein) other than PSMA.

As used herein, the term “antibody” refers to a protein comprising at least one, and preferably two, heavy (H) chain variable regions (abbreviated herein-as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Preferably, each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or from a sequence generated by phage display, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., a mouse, framework modified to decrease antigenicity in humans, or a synthetic framework, e.g., a consensus sequence. These are sometimes referred to herein as modified immunoglobulins. A modified antibody, or antigen binding fragment thereof, includes at least one, two, three or four modified immunoglobulin chains, e.g., at least one or two modified immunoglobulin light and/or at least one or two modified heavy chains. In one embodiment, the modified antibody is a tetramer of two modified heavy immunoglobulin chains and two modified light immunoglobulin chains.

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to a portion of an antibody which specifically binds to PSMA (e.g., human PSMA), e.g., a molecule in which one or more immunoglobulin chains is not full length but which specifically binds to PSMA (e.g., human PSMA protein). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated (e.g., by phage display) antibodies, and may optionally include constant regions derived from human germline immunoglobulin sequences.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a homology limitation of the invention) are a Blossum 62 scoring matrix with a gap penalty of 12, a gap extend penalty of 4, and a frameshift gap penalty of 5.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the binding agent polypeptides of the invention may have additional conservative or non-essential amino acid substitutions, which do not have a substantial effect on the polypeptide functions. Whether or not a particular substitution will be tolerated, i.e., will not adversely affect desired biological properties, such as binding activity can be determined as described in Bowie, J U et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

Anti-PSMA Antibodies

Many types of anti-PSMA antibodies, or antigen-binding fragments thereof, are useful in the methods of this invention. The antibodies can be of the various isotypes, including: IgG (e.g., IgG1, IgG2, IgG3, IgG4), IgM, IgA1, IgA2, IgD, or IgE. Preferably, the antibody is an IgG isotype. The antibody molecules can be full-length (e.g., an IgG1 or IgG4 antibody) or can include only an antigen-binding fragment (e.g., a Fab, F(ab′)₂, Fv or a single chain Fv fragment). These include monoclonal antibodies, recombinant antibodies, chimeric antibodies, humanized antibodies, deimmunized antibodies, as well as antigen-binding fragments of the foregoing.

As described in more detail below, antibodies (preferably, monoclonal antibodies from differing organisms, e.g., rodent, sheep, human) against a predetermined antigen can be produced using art-recognized methods. Once the antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions. A light and the heavy immunoglobulin chains can be generated and co-expressed into the appropriate host cells.

Monoclonal anti-PSMA antibodies can be used in the methods of the invention. Preferably, the monoclonal antibodies bind to the extracellular domain of PSMA (i.e., an epitope of PSMA located outside of a cell). Examples of preferred murine monoclonal antibodies to human PSMA include, but are not limited to, E99, J415, J533 and J591, which are produced by hybridoma cell lines having an ATCC Accession Number HB-12101, HB-12109, HB-12127, and HB-12126, respectively, all of which are disclosed in U.S. Pat. No. 6,107,090 and U.S. Pat. No. 6,136,311, the contents of which are expressly incorporated by reference. Most preferably, the murine monoclonal antibody is J591, produced by HB-12126.

Additional monoclonal antibodies to PSMA can be generated using techniques known in the art. Monoclonal antibodies can be produced by a variety of techniques, including conventional monoclonal antibody methodology e.g., the standard somatic cell hybridization technique of Kohler and Milstein, Nature 256: 495 (1975). See generally, Harlow, E. and Lane, D. (1988) Antibodies: A Laboratory Manual, Cold Spring Harbor Laboratory Press, Cold Spring Harbor, N.Y. Although somatic cell hybridization procedures are preferred, in principle, other techniques for producing monoclonal antibody can be employed e.g., viral or oncogenic transformation of B lymphocytes. The preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Useful immunogens for the purpose of this invention include PSMA (e.g., human PSMA)-bearing cells (e.g., dermal or epidermal cells from a subject with psoriasis or prostate tumor cell lines, e.g., LNCap cells); isolated or purified PSMA, e.g., human PSMA, e.g., biochemically isolated PSMA, or a portion thereof, e.g., the extracellular domain of PSMA. Techniques for generating antibodies to PSMA are described in U.S. Pat. No. 6,107,090, U.S. Pat. No. 6,136,311, the contents of all of which are expressly incorporated by reference.

Human monoclonal antibodies (mAbs) directed against human proteins can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Anti-PSMA antibodies or fragments thereof useful in the present invention may also be recombinant antibodies produced by host cells transformed with DNA encoding immunoglobulin light and heavy chains of a desired antibody. Recombinant antibodies may be produced by known genetic engineering techniques. For example, recombinant antibodies may be produced by cloning a nucleotide sequence, e.g., a cDNA or genomic DNA sequence, encoding the immunoglobulin light and heavy chains of the desired antibody from a hybridoma cell that produces an antibody useful in this invention. The nucleotide sequence encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector. Prokaryotic or eukaryotic host cells may be used.

Expression in eukaryotic host cells is preferred because such cells are more likely than prokaryotic cells to assemble and secrete a properly folded and immunologically active antibody. However, any antibody produced that is inactive due to improper folding may be renaturable according to well known methods (Kim and Baldwin, “Specific Intermediates in the Folding Reactions of Small Proteins and the Mechanism of Protein Folding”, Ann. Rev. Biochem. 51, pp. 459-89 (1982)). It is possible that the host cells will produce portions of intact antibodies, such as light chain dimers or heavy chain dimers, which also are antibody homologs according to the present invention.

It will be understood that variations on the above procedure are useful in the present invention. For example, it may be desired to transform a host cell with DNA encoding either the light chain or the heavy chain (but not both) of an antibody. Recombinant DNA technology may also be used to remove some or all of the DNA encoding either or both of the light and heavy chains that is not necessary for PSMA binding, e.g., the constant region may be modified by, for example, deleting specific amino acids. The molecules expressed from such truncated DNA molecules are useful in the methods of this invention. In addition, bifunctional antibodies may be produced in which one heavy and one light chain are anti-PSMA antibody and the other heavy and light chain are specific for an antigen other than PSMA, or another epitope of PSMA.

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

An antibody or an immunoglobulin chain can be humanized by methods known in the art. Once the murine antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions.

Murine anti-PSMA antibodies can be sequenced using art-recognized techniques. As an example, hybridomas expressing murine antibodies J533, J415 and E99 were propagated in culture in RPMI 1640 medium supplemented with 10% fetal calf serum. The isotype of the antibodies secreted was confirmed as IgG1κ, IgG1κ, and IgG3κ respectively. These monoclonal antibodies, like J591, bind to the external domain of prostate specific membrane antigen. J591, J533 and E99 recognize the same epitope, while J415 recognizes an independent epitope. Total RNA for each monoclonal was prepared from 107 hybridoma cells. V_{H} and V_{K} cDNA was prepared using reverse transcriptase and mouse K constant region and mouse IgG constant region primers. The first strand cDNAs were amplified by PCR using a variety of mouse signal sequence primers (6 for V_{H} and 7 for V_{K}). The amplified DNAs were gel-purified and cloned into the vector pT7Blue. The V_{H} and V_{K} clones obtained were screened for correct inserts by PCR and the DNA sequence of selected clones determined by the dideoxy chain termination method (see Table 1).

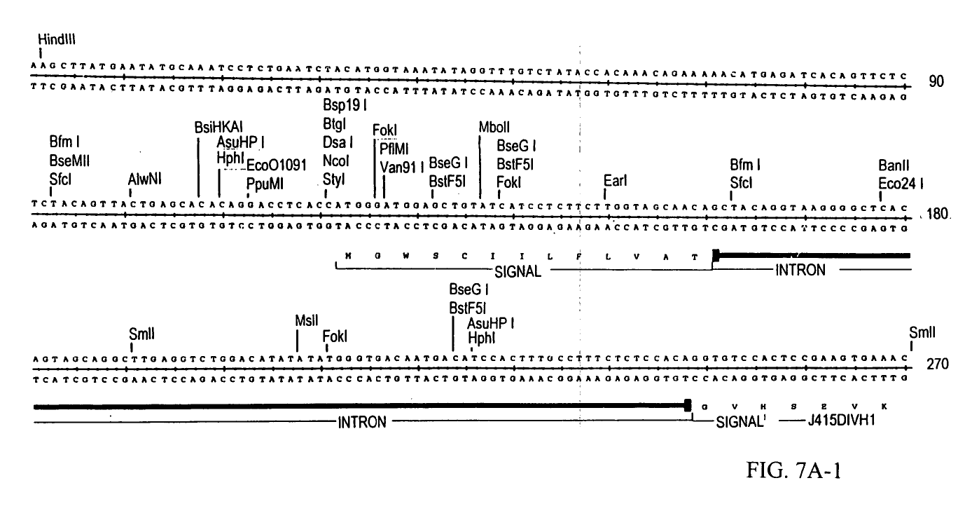

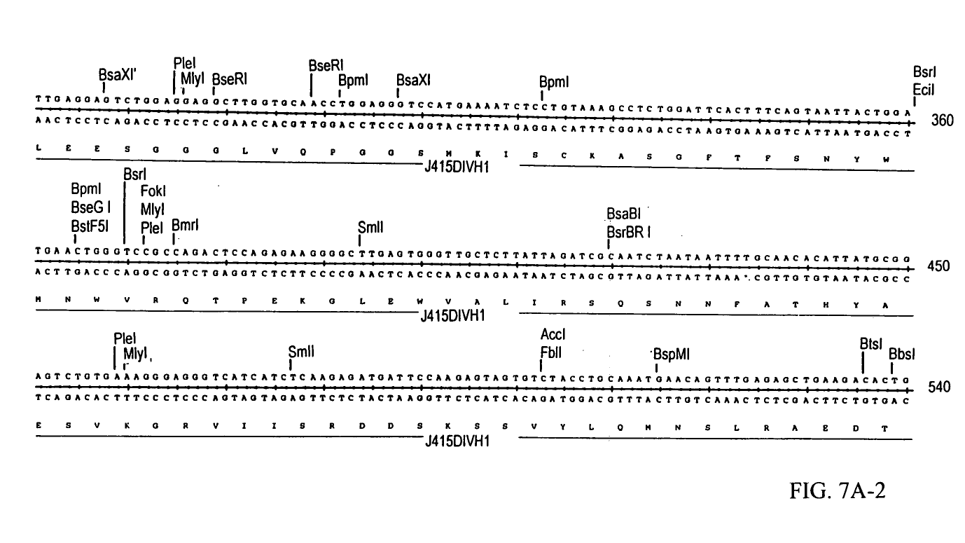

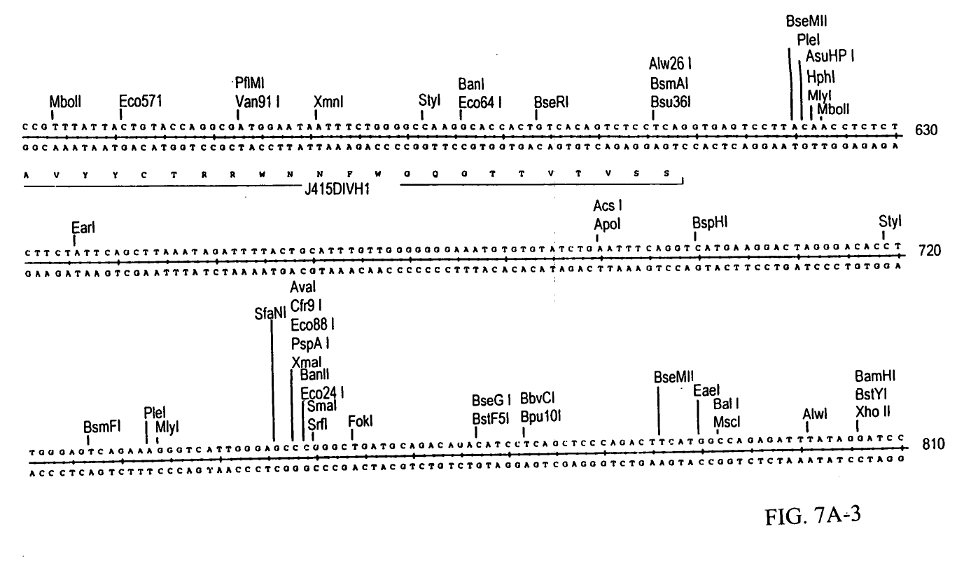

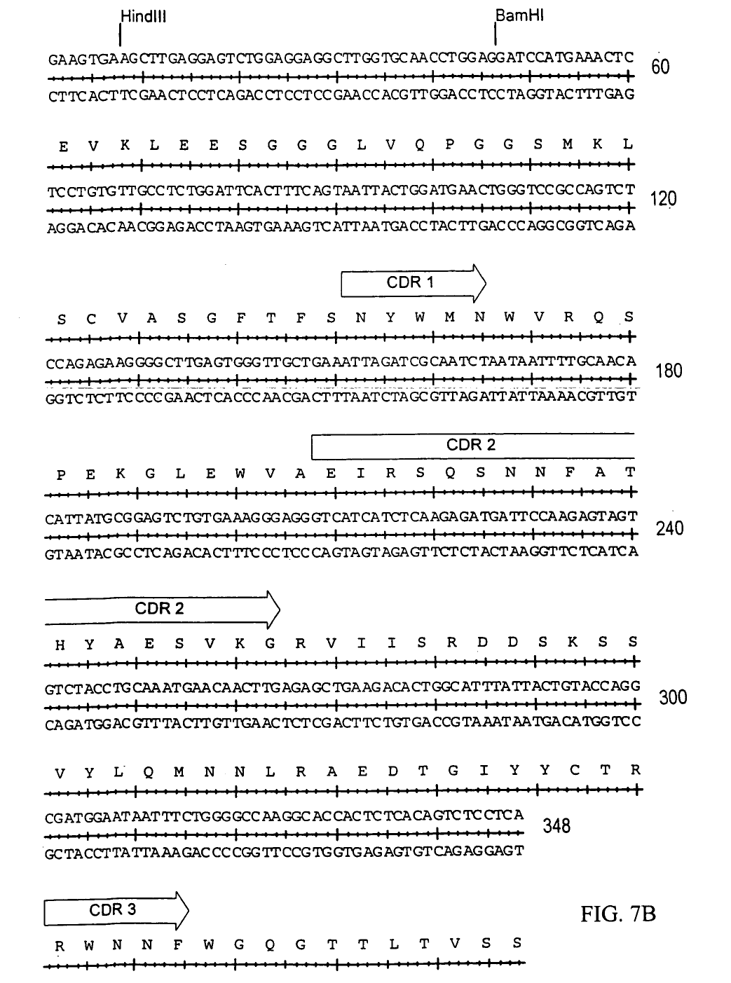

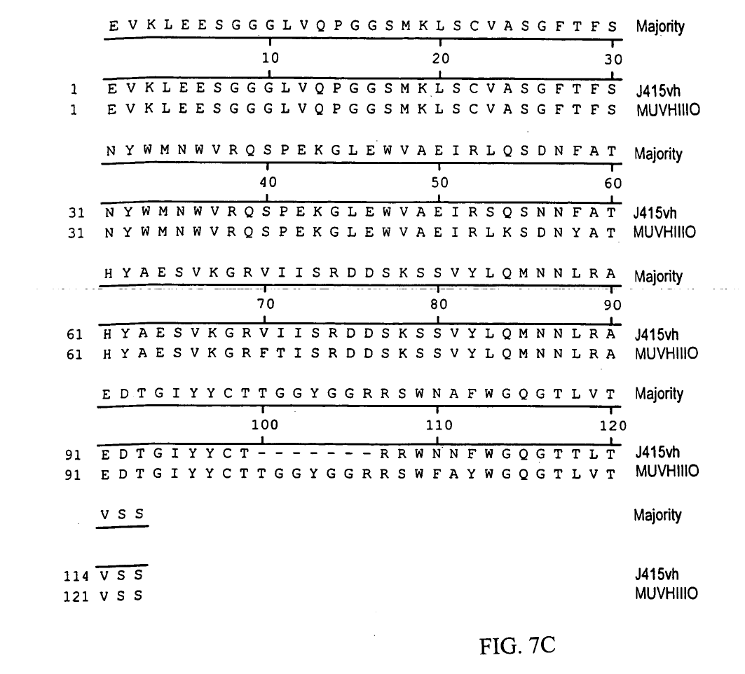

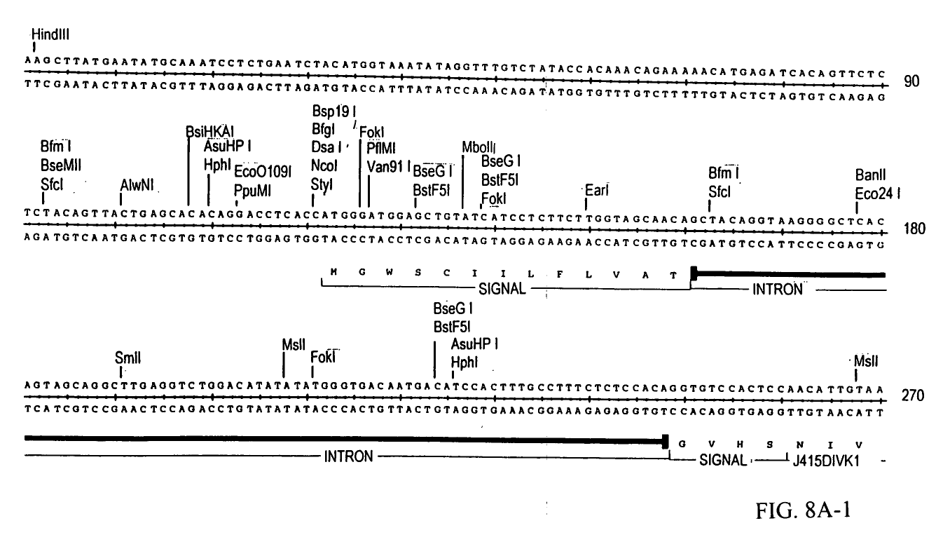

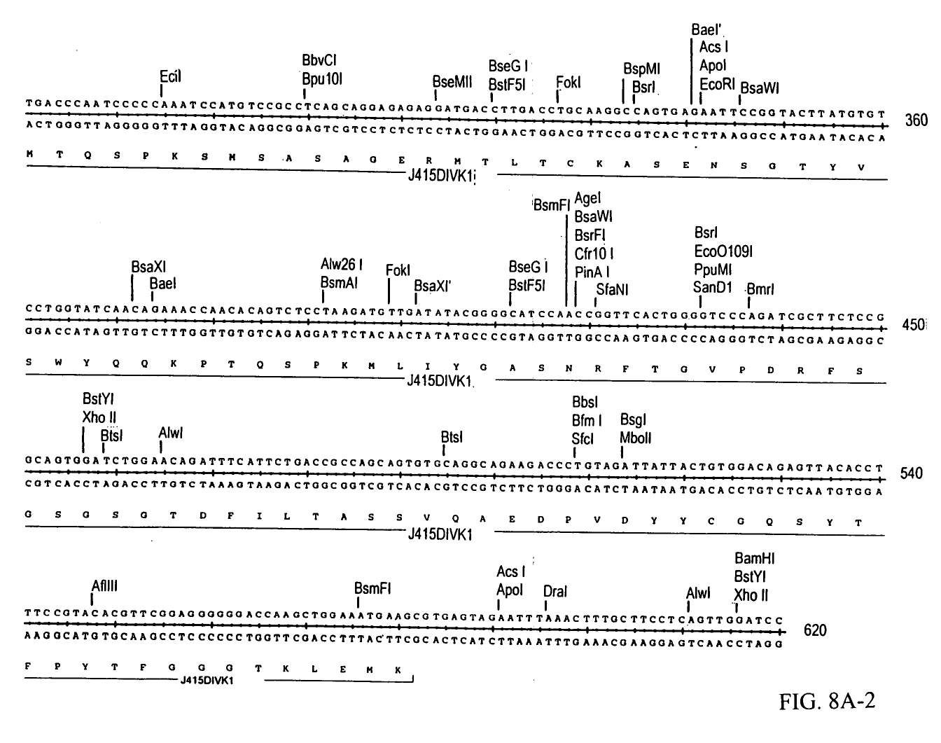

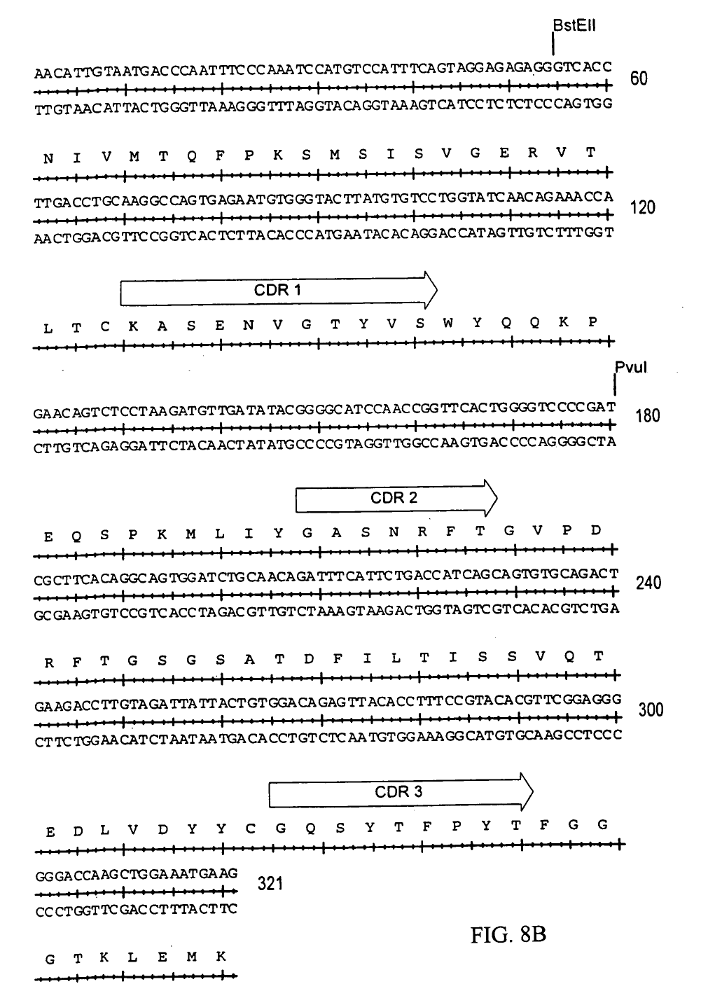

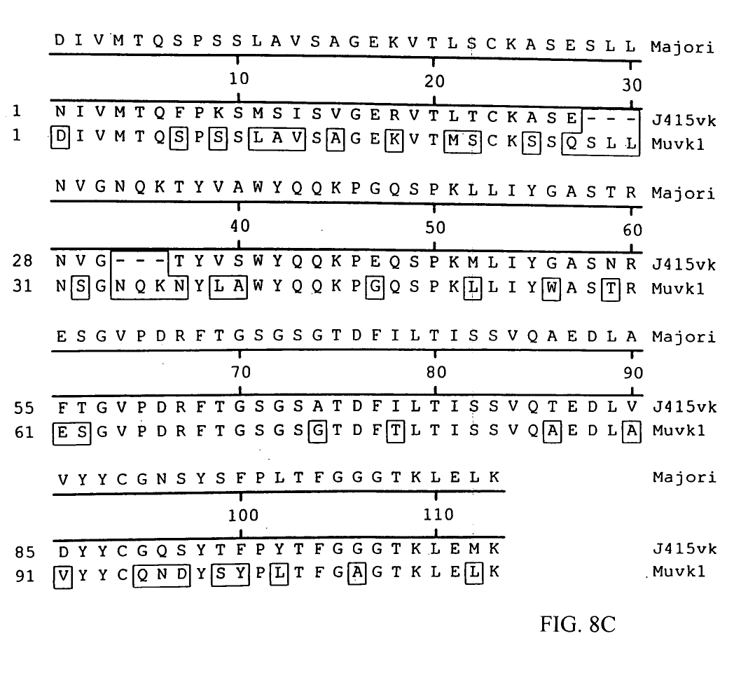

The DNA and amino acid sequences for the heavy and light chain variable regions from J415 were obtained and are shown in FIGS. 7B (V_{H}) and 8B (V_{K}) (also, see Table 1). The location of the CDRs is shown. J415 VH can be assigned to Mouse Heavy Chains Subgroup IIIC (Kabat E A et al; ibid). The sequence of J415 VH compared to the consensus sequence for this subgroup is shown in FIG. 7C. J415 VK can be assigned to Mouse Kappa Chains Subgroup I (Kabat E A et al; ibid). The sequence of J415 VK compared to the consensus sequence for this subgroup is shown in FIG. 8C.

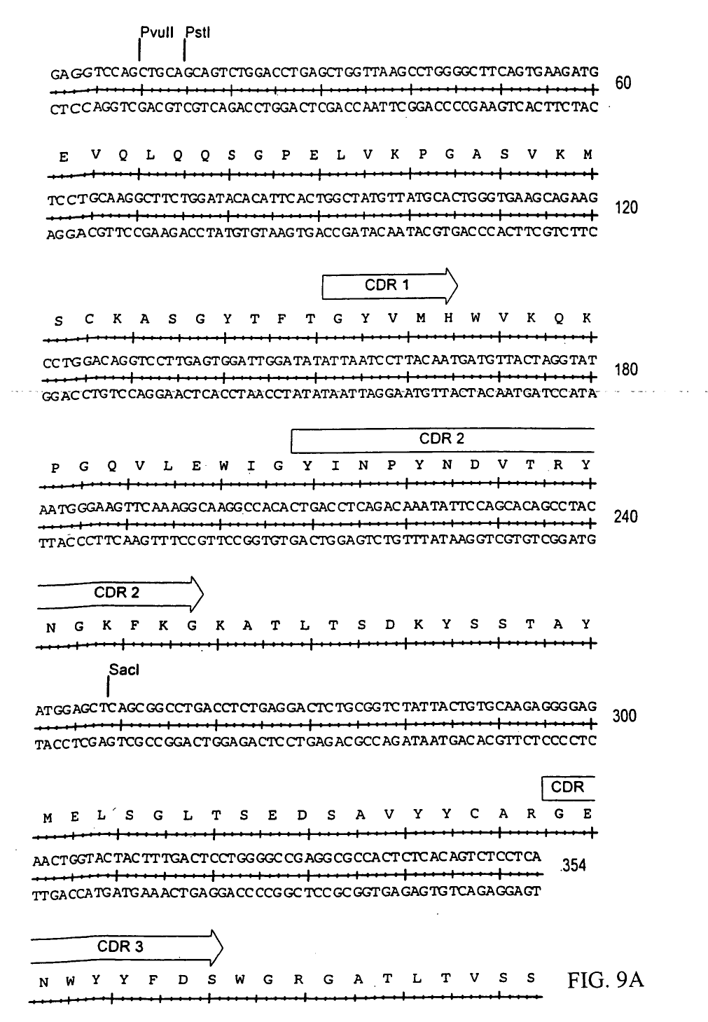

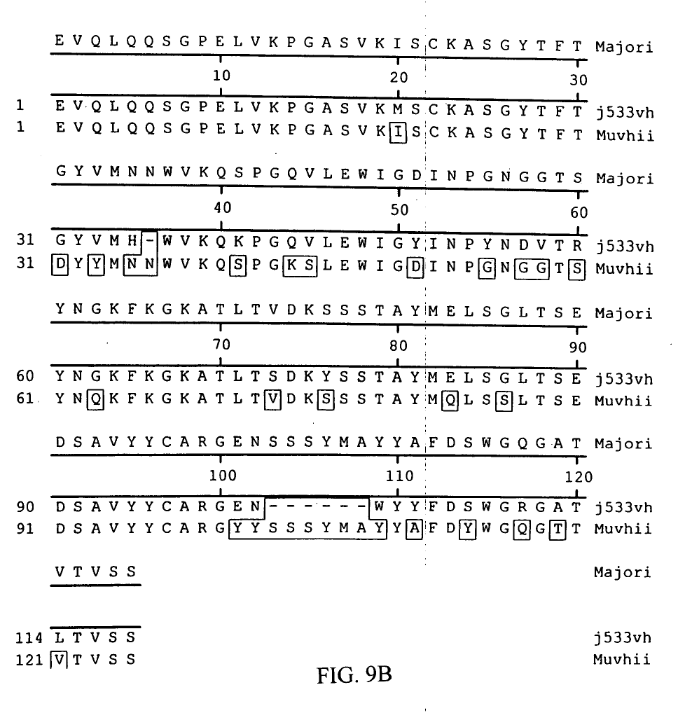

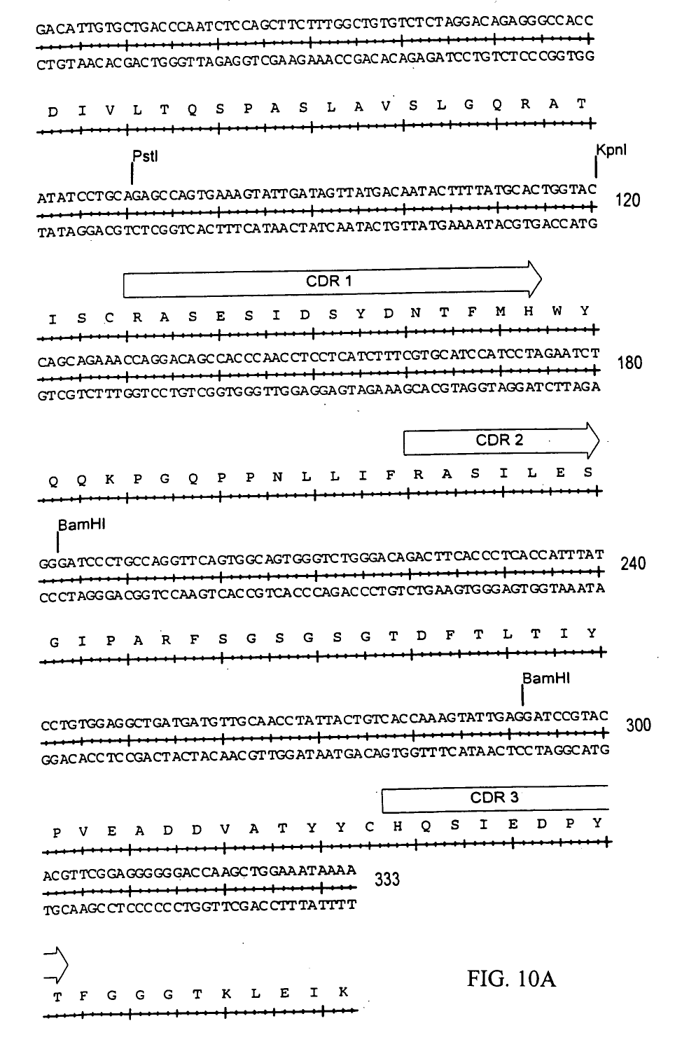

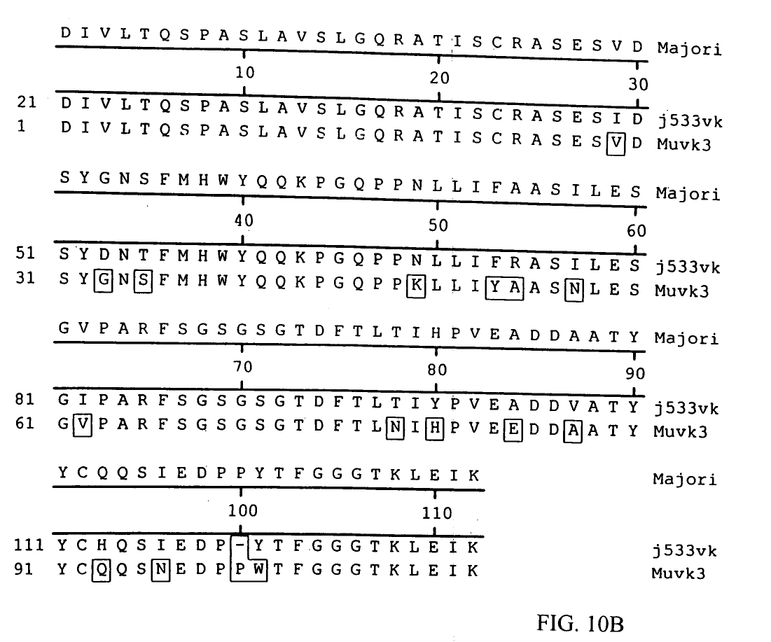

The DNA and amino acid sequences encoding the heavy and light chain variable regions of J533 were obtained and are shown in FIGS. 9A (V_{H}) and 10A (V_{K}) (see also Table 1). The location of the CDRs is shown in each figure. J533 V_{H} can be assigned to Mouse Heavy Chains Subgroup IIA (Kabat E A et al; Sequences of proteins of Immunological Interest, US Department of Health and Human Services, 1991). The sequence of J533 V_{H} compared to the consensus sequence for this subgroup is shown in FIG. 9B. J533 V_{K} can be assigned to Mouse Kappa Chains Subgroup III (Kabat E A et al; ibid). The sequence of J533 V_{K} compared to the consensus sequence for this subgroup is shown in FIG. 10B.

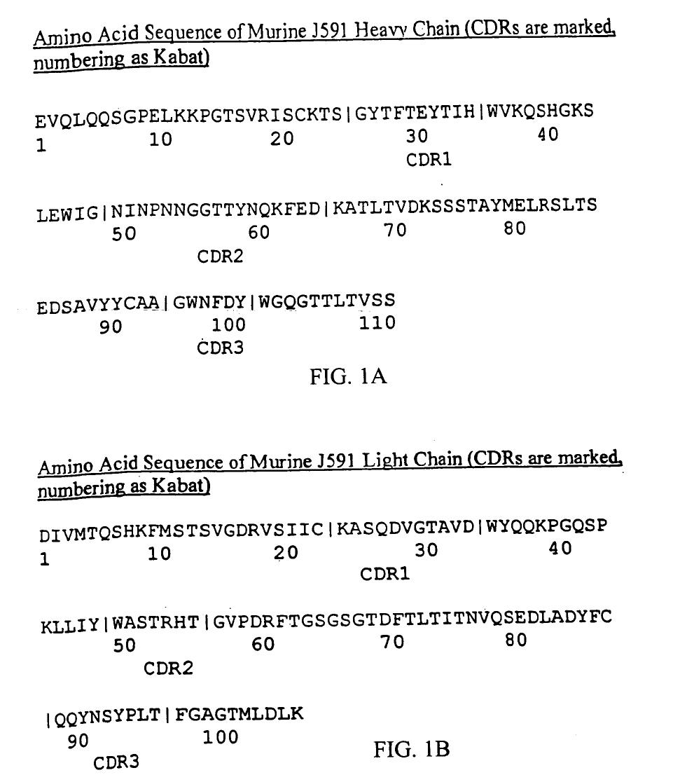

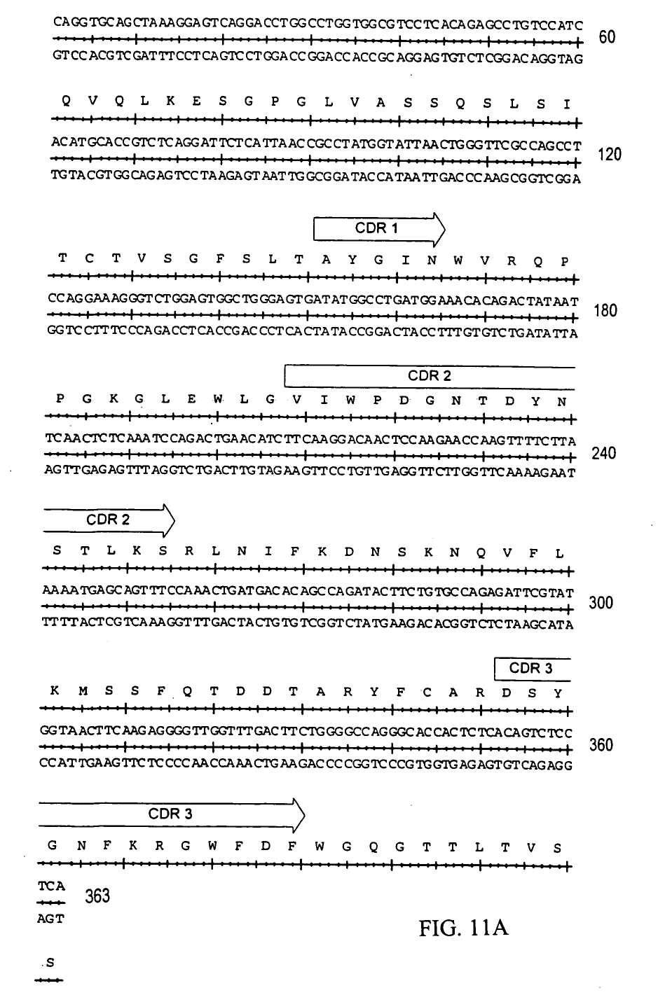

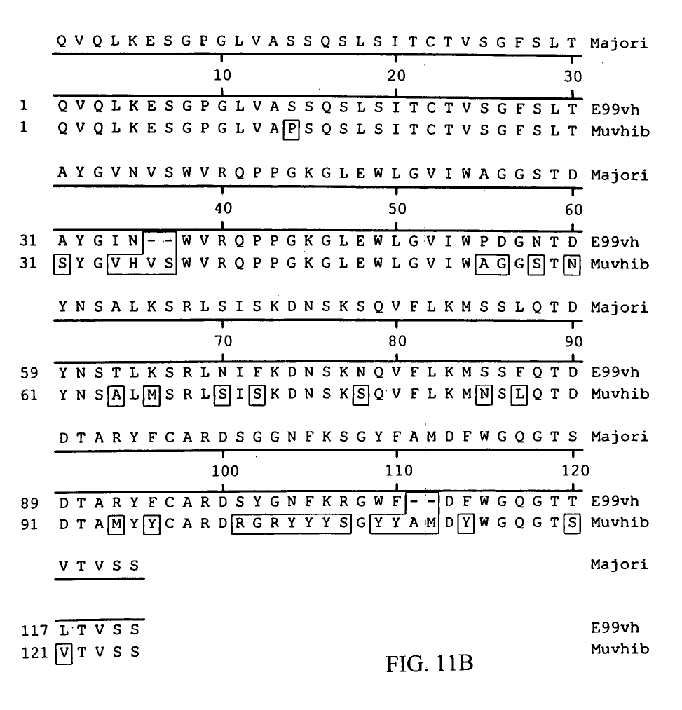

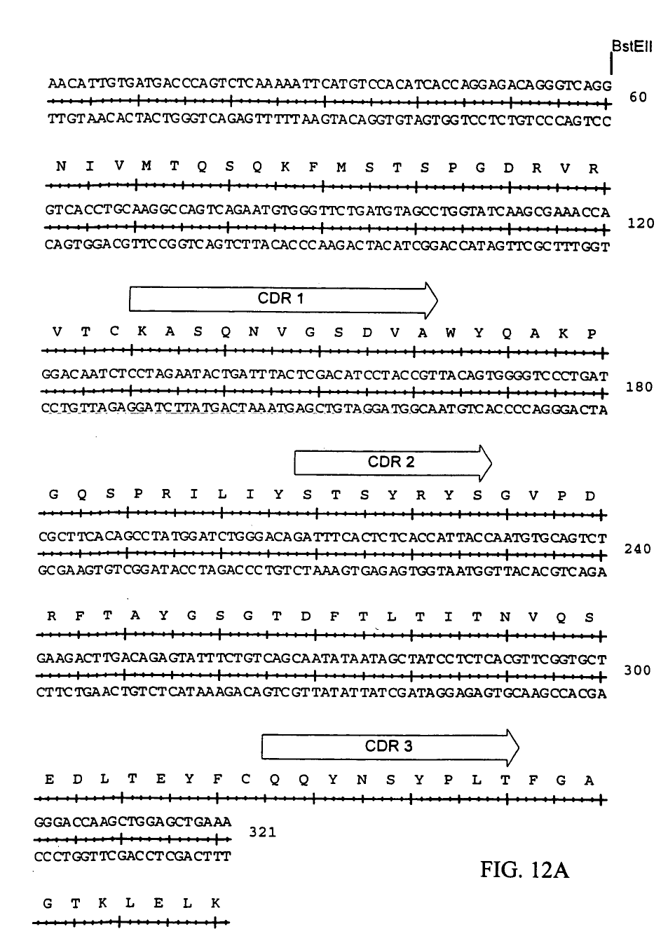

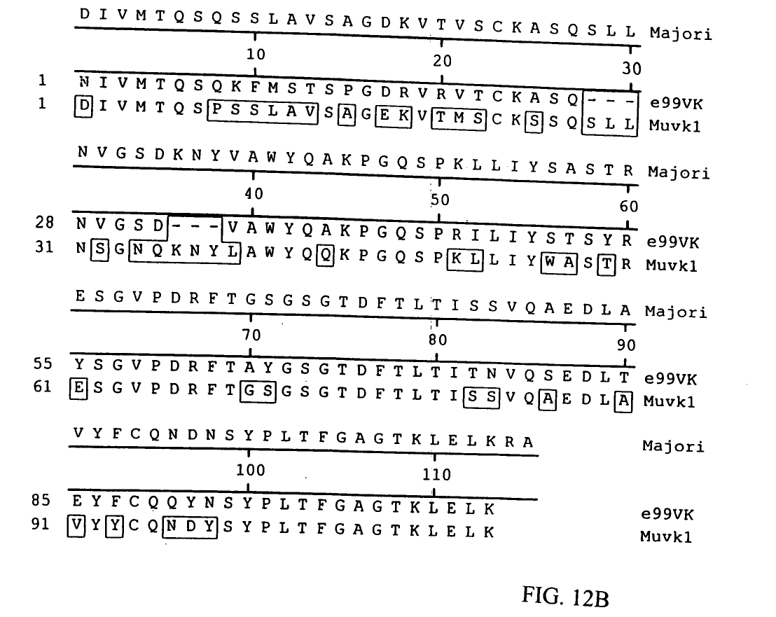

The DNA and amino acid sequences of the heavy and light chain variable regions of E99 were obtained and are shown in FIGS. 11A (V_{H}) and 12A (V_{K}) (see also Table 1). The location of the CDRs is shown. E99 V_{H} can be assigned to Mouse Heavy Chains Subgroup IB (Kabat E A et al; ibid). The sequence of E99 V_{H} compared to the consensus sequence for this subgroup is shown in FIG. 1B. E99 V_{K} can be assigned Mouse Kappa Chains Subgroup I (Kabat E A et al; ibid). The sequence of E99 V_{K} compared to the consensus sequence for this subgroup is shown in FIG. 12B.

The amino acid sequence and nucleotide sequences encoding the variable region of antibodies J415, deJ415, J591, deJ591, J533 and E99 are provided below in Table 1.

TABLE 1
Antibody variable chain sequences

| | | | | |
|---|---|---|---|---|
| | | | SEQ | |
| | | | ID | |
| NAME | Organism | FIG. | NO: | SEQUENCE |

| | | | | |
|---|---|---|---|---|
| V_{H} J415 | Mus musculus | | 125 | gaagtgaagcttgaggagtctggaggaggcttggtgcaacctgg |
| | | | | aggatccatgaaactctcctgtgttgcctctggattcactttcagtaat |
| | | | | tactggatgaactgggtccgccagtctccagagaaggggcttgag |
| | | | | tgggttgctgaaattagatcgcaatctaataattttgcaacacattatg |
| | | | | cggagtctgtgaaagggagggtcatcatctcaagagatgattcca |
| | | | | agagtagtgtctacctgcaaatgaacaacttgagagctgaagaca |
| | | | | ctggcatttattactgtaccaggcgatggaataatttctggggccaa |
| | | | | ggcaccactctcacagtctcctca |
| V_{H} Variable | Mus musculus | | 19 | EVQLQQSGPELKKPGTSVRISCKTSGYTFT |
| Region | | | | EYTIHWVKQSHGKSLEWIGNTNPNNGGTT |
| J591 | | | | YNQKFEDKATLTVDKSSSTAYMELRSLTS |
| | | | | EDSAVYYCAAGWNFDYWGQGTTLTVSS |
| V_{H} J415 | Mus musculus | | 126 | Tgaggagactgtgagagtggtgccttggccccagaaattattcca |
| (complementary | | | | tcgcctggtacagtaataaatgccagtgtcttcagctctcaagttgtt |
| strand of | | | | catttgcaggtagacactactcttggaatcatctcttgagatgatgac |
| SEQ ID NO:125 | | | | cctccctttcacagactccgcataatgtgttgcaaaattattagattg |
| | | | | cgatctaatttcagcaacccactcaagccccttctctggagactgg |
| | | | | cggacccagttcatccagtaattactgaaagtgaatccagaggca |
| | | | | acacaggagagtttcatggatcctccaggttgcaccaagcctcctc |
| | | | | cagactcctcaagcttcacttc |
| V_{L} J415 | Mus musculus | | 127 | aacattgtaatgacccaatttcccaaatccatgtccatttcagtagga |
| | | | | gagagggtcaccttgacctgcaaggccagtgagaatgtgggtact |
| | | | | tatgtgtcctggtatcaacagaaaccagaacagtctcctaagatgtt |
| | | | | gatatacggggcatccaaccggttcactggggtccccgatcgctt |
| | | | | cacaggcagtggatctgcaacagatttcattctgaccatcagcagt |
| | | | | gtgcagactgaagaccttgtagattattactgtggacagagttacac |
| | | | | ctttccgtacacgttcggaggggggaccaagctggaaatgaag |
| V_{L} Variable | Mus musculus | | 20 | DIVMTQSHKFMSTSVGDRVSIIGKASQDV |
| Region | | | | GTAVDWYQQKPGQSPKLLIYWASTRHTG |
| J591 | | | | VPDRFTGSGSGTDFTLTITNVQSEDLADYF |
| | | | | CQQYNSYPLTFGAGTMLDLK |
| V_{L} J415 | Mus musculus | | 128 | cttcatttccagcttggtcccccctccgaacgtgtacggaaaggtgt |
| (complementary | | | | aactctgtccacagtaataatctacaaggtcttcagtctgcacactg |
| strand of | | | | ctgatggtcagaatgaaatctgttgcagatccactgcctgtgaagc |
| SEQ ID NO:127) | | | | gatcggggaccccagtgaaccggttggatgccccgtatatcaaca |
| | | | | tcttaggagactgttctggtttctgttgataccaggacacataagtac |
| | | | | ccacattctcactggccttgcaggtcaaggtgaccctctctcctact |
| | | | | gaaatggacatggatttgggaaattgggtcattacaatgtt |
| V_{H} Variable | Artificial-deimmunized | | 21 | EVQLVQSGPEVKKPGATVKISCKTSGYTFT |
| Region | | | | EYTLHWVKQAPGKGLEWIGNINPNNGGTT |
| (Deimm) | heavy chain | | | YNQKFEDKATLTVDKSTDTAYMELSSLRS |
| J591 | J591 | | | EDTAVYYCAAGWNFDYWGQGTLLTVSS |
| V_{L} Variable | Artificial-deimmunized | | 22 | DIQMTQSPSSLSTSVGDRVTLTCKASQDV |
| Region | | | | GTAVDWYQQKPGPSPKLLIYWASTRIITGI |
| (Deimm) | light chain | | | PSRFSGSGSGTDFTLTISSLQPEDFADYYCQ |
| J591 | J591 | | | QYNSYPLTFGPGTKVDLK |
| V_{H} | Artificial-deimmunized | | 23 | Aagcttatgaatatgcaaatcctctgaatctacatggtaaatatagg |
| Deimmunized | | | | tttgtctataccacaaacagaaaaacatgagatcacagttctctctac |
| J591 | heavy chain | | | agttactgagcacacaggacctcaccatgggatggagctgtatcat |
| | J591 | | | cctcttcttggtagcaacagctacaggtaaggggctcacagtagca |
| CDS(122-166) | | | | ggcttgaggtctggacatatatatgggtgacaatgacatccactttg |
| & | | | | cctttctctccacaggtgtccactccgaggtccaactggtacagtct |
| CDS (249-605) | | | | ggacctgaagtgaagaagcctggggctacagtgaagatatcctg |
| | | | | caagacttctggatacacattcactgaatataccatacactgggtga |
| | | | | agcaggcccctggaaagggccttgagtggattggaaacatcaatc |
| | | | | ctaacaatggtggtaccacctacaatcagaagttcgaggacaagg |
| | | | | ccacactaactgtagacaagtccaccgatacagcctacatggagc |
| | | | | tcagcagcctaagatctgaggatactgcagtctattattgtgcagct |
| | | | | ggttggaactttgactactggggccaagggaccctgctcaccgtct |
| | | | | cctcaggtgagtccttacaacctctctcttctattcagcttaaatagat |
| | | | | tttactgcatttgttgggggggaaatgtgtgtatctgaatttcaggtca |
| | | | | tgaaggactagggacaccttgggagtcagaaagggtcattggga |
| | | | | gcccgggctgatgcagacagacatcctcagctcccagacttcatg |
| | | | | gccagagatttataggatcc |
| V_{H} | Artificial-deimmunized | | 24 | ggatcctataaatctctggccatgaagtctgggagctgaggatgtc |
| Deimmunized | | | | tgtctgcatcagcccgggctcccaatgaccctttctgactcccaag |
| (complementary | heavy chain | | | gtgtccctagtccttcatgacctgaaattcagatacacacatttcccc |
| strand of | J591 | | | cccaacaaatgcagtaaaatctatttaagctgaatagaagagagag |
| SEQ ID NO:23) | | | | gttgtaaggactcacctgaggagacggtgagcagggtcccttggc |
| | | | | cccagtagtcaaagttccaaccagctgcacaataatagactgcagt |
| J591 | | | | atcctcagatcttaggctgctgagctccatgtaggctgtatcggtgg |
| | | | | acttgtctacagttagtgtggccttgtcctcgaacttctgattgtaggt |
| | | | | ggtaccaccattgttaggattgatgtttccaatccactcaaggccctt |
| | | | | tccaggggcctgcttcacccagtgtatggtatattcagtgaatgtgt |
| | | | | atccagaagtcttgcaggatatcttcactgtagccccaggcttcttc |
| | | | | acttcaggtccagactgtaccagttggacctcggagtggacacct |
| | | | | gtggagagaaaggcaaagtggatgtcattgtcacccatatatatgt |
| | | | | ccagacctcaagcctgctactgtgagccccttacctgtagctgttgc |
| | | | | taccaagaagaggatgatacagctccatcccatggtgaggtcctgt |
| | | | | gtgctcagtaactgtagagagaactgtgatctcatgtttttctgtttgt |
| | | | | ggtatagacaaacctatatttaccatgtagattcagaggatttgcata |
| | | | | ttcataagctt |
| V_{L} | Artificial-deimmunized | | 25 | aagcttatgaatatgcaaatcctctgaatctacatggtaaatataggt |
| Deimmunized | | | | ttgtctataccacaaacagaaaaacatgagatcacagttctctctac |
| J591 | light chain | | | agttactgagcacacaggacctcaccatgggatggagctgtatcat |
| | J591 | | | cctcttcttggtagcaacagctacaggtaaggggctcacagtagca |
| CDS (122-166) | | | | ggcttgaggtctggacatatatatgggtgacaatgacatccactttg |
| & | | | | cctttctctccacaggtgtccactccgacatccagatgacccagtct |
| CDS (249-581) | ccctcatccctgtccacatcagtaggagacagggtcaccctcacct | | | |
| | | | | gtaaggccagtcaagatgtgggtactgctgtagactggtatcaaca |
| | | | | gaaaccaggaccatctcctaaactactgatttattgggcatccactc |
| | | | | ggcacactggaatccctagtcgcttctcaggcagtggatctggga |
| | | | | cagacttcactctcaccatttctagtcttcagcctgaagactttgcag |
| | | | | attattactgtcagcaatataacagctatcctctcacgttcggtcctg |
| | | | | ggaccaaggtggacatcaaacgtgagtagaatttaaactttgcttc |
| | | | | ctcagttggatcc |
| V_{L} | Artificial-deimmunized | | 26 | ggatccaactgaggaagcaaagtttaaattctactcacgtttgatgt |
| Deimmunized | | | | ccaccttggtcccaggaccgaacgtgagaggatagctgttatattg |
| (complimentar | light chain | | | ctgacagtaataatctgcaaagtcttcaggctgaagactagaaatg |
| y strand of | J591 | | | gtgagagtgaagtctgtcccagatccactgcctgagaagcgacta |
| SEQ ID NO:25) | | | | gggattccagtgtgccgagtggatgcccaataaatcagtagtttag |
| | | | | gagatggtcctggtttctgttgataccagtctacagcagtacccaca |
| J591 | | | | tcttgactggccttacaggtgagggtgaccctgtctcctactgatgt |
| | | | | ggacagggatgagggagactgggtcatctggatgtcggagtgga |
| | | | | cacctgtggagagaaaggcaaagtggatgtcattgtcacccatata |
| | | | | tatgtccagacctcaagcctgctactgtgagccccttacctgtagct |
| | | | | gttgctaccaagaagaggatgatacagctccatcccatggtgagg |
| | | | | tcctgtgtgctcagtaactgtagagagaactgtgatctcatgtttttct |
| | | | | gtttgtggtatagacaaacctatatttaccatgtagattcagaggattt |
| | | | | gcatattcataagctt |
| V_{H} | Artificial-deimmunized | | 27 | MGWSCIILFLVATATGVHSEVQLVQSGPE |
| Deimmunized | | | | VKKPGATVKISCKTSGYTFTEYTIIHWVKQ |
| (predicted a.a. | heavy chain | | | APGKGLEWIGNLNPNNGGTTYNQKFEDKA |
| of SEQ ID NO:23) | J591 | | | TLTVDKSTDTAYMELSSLRSEDTAVYYCA |
| | | | | AGWNFDYWGQGTLLTVSS |
| J591 | | | | |
| V_{L} | Artificial-deimmunized | | 28 | MGWSCIILFLVATATGVHSDIQMTQSPSSL |
| Deimmunized | | | | STSVGDRVTLTCKASQDVGTAVDWYQQK |
| (predicted a.a. | light chain | | | PGPSPKLLIYCASTRHTGLPSRFSGSGSGTD |
| of SEQ ID NO:25) | J591 | | | FTLTISSLQPEDFADYYGQQYNSYPLTFGP |
| | | | | GTKVDIK |
| J591 | | | | |
| V_{H} Variable | Mus musculus | | 47 | EVKLEESGGGLVQPGGSMKLSCVASGFTF |
| Region | | | | SNYWMNWVRQSPEKGLEWVAEIRSQSNN |
| J415 | | | | FATHYAESVKGRVIISRDDSKSSVYLQMN |
| | | | | NLRAEDTG1YYCTRRWNNFWGQGTTLTV |
| | | | | SS |
| V_{L} Variable | Mus musculus | | 48 | NIVMTQFPKSMSISVGERVTLTCKASENVG |
| Region | | | | TYVSWYQQKPEQSPKMLWGASNRPTGVP |
| J415 | | | | DRFTGSGSATDFILTISSVQTEDLVDYYCG |
| | | | | QSYTFPYTFGGGTKLEMK |
| V_{H} Variable | Artificial-deimmunized | | 49 | EVKLEESGGGLVQPGGSMKISCVASGFTFs |
| Region | | | | NYWMNWVRQSPEKGLEWVAEIRSQSNNF |
| (Deimm) | heavy chain | | | ATHYAESVKGRVIISRDDSKSSVYLQMNS |
| J415-4 | J415-4 | | | LRAEDTAVYYCTRRWNNFWGQGTTVTVS |
| | | | | S |
| V_{L} Variable | Artificial-deimmunized | | 50 | N1VMTQFPKSMSASAGERMTLTGKASENV |
| Region | | | | GTYVSWYQQKPTQSPKMLIYGASNRFTGV |
| (Deimm) | light chain | | | PDRFSGSGSGTDFILTISSVQAEDLVDYYC |
| J415-5 | J415-5 | | | GQSYTFPYTFGGGTKLEMK |
| V_{H} | Artificial-deimmunized | | 51 | gaagtgaaacttgaggagtctggaggaggcttggtgcaacctgg |
| Deimmunized | | | | agggtccatgaaaatctcctgtgttgcctctggattcactttcagtaa |
| J415-4 | heavy chain | | | ttactggatgaactgggtccgccagtctccagagaaggggcttga |
| J4 | 15-4 | | | gtgggttgctgaaattagatcgcaatctaataattttgcaacacattat |
| | | | | gcggagtctgtgaaagggagggtcatcatctcaagagatgattcc |
| | | | | aagagtagtgtctacctgcaaatgaacagtttgagagctgaagaca |
| | | | | ctgccgtttattactgtaccaggcgatggaataatttctggggccaa |
| | | | | ggcaccactgtcacagtctcctca |
| V_{L} | Artificial-deimmunized | | 52 | aacattgtaatgacccaatttcccaaatccatgtccgcctcagcagg |
| Deimmunized | | | | agagaggatgaccttgacctgcaaggccagtgagaatgtgggta |
| J415-5 | light chain | | | cttatgtgtcctggtatcaacagaaaccaacacagtctcctaagatg |
| J415-5 | | | | ttgatatacggggcatccaaccggttcactggggtcccagatcgct |
| | | | | tctccggcagtggatctggaacagatttcattctgaccatcagcagt |
| | | | | gtgcaggcagaagaccttgtagattattactgtggacagagttaca |
| | | | | cctttccgtacacgttcggaggggggaccaagctggaaatgaag |
| V_{H} | Artificial-deimmunized | | 53 | aagcttatgaatatgcaaatcctctgaatctacatggtaaatataggt |
| Deimmunized | | | | ttgtctataccacaaacagaaaaacatgagatcacagttctctctac |
| J415-1 | heavy chain | | | agttactgagcacacaggacctcaccatgggatggagctgtatca |
| | J415-1 | | | tcctcttcttggtagcaacagctacaggtaaggggctcacagtagc |
| CDS(122-160) | | | | aggcttgaggtctggacatatatatgggtgacaatgacatccacttt |
| & | | | | gcctttctctccacaggtgtccactccgaagtgaaacttgaggagt |
| CDS(249-608) | | | | ctggaggaggcttggtgcaacctggagggtccatgaaaatctcct |
| | | | | gtaaagcctctggattcactttcagtaattactggatgaactgggtcc |
| | | | | gccagactccagagaaggggcttgagtgggttgctcttattagatc |
| | | | | gcaatctaataattttgcaacacattatgcggagtctgtgaaaggga |
| Mature (18-133) | | | | gggtcatcatctcaagagatgattccaagagtagtgtctacctgca |
| | | | | aatgaacagtttgagagctgaagacactgccgtttattactgtacca |
| | | | | ggcgatggaataatttctggggccaaggcaccactgtcacagtct |
| | | | | cctcaggtgagtccttacaacctctctcttctattcagcttaaatagat |
| | | | | tttactgcatttgttgggggggaaatgtgtgtatctgaatttcaggtca |
| | | | | tgaaggactagggacaccttgggagtcagaaagggtcattggga |
| | | | | gcccgggctgatgcagacagacatcctcagctcccagacttcatg |
| | | | | gccagagatttataggatcc |
| V_{H} | Artificial-deimmunized | | 54 | MGWSCIILFLVATGVHSEVKLEESGGGLV |
| Deimmunized | | | | QPGGSMKISCKASGFTFSNYWMNWVRQT |
| (predicted a.a. | heavy chain | | | PEKGLEWVALIRSQSNNEATHYAESVKGR |
| of SEQ ID NO:53) | J415-1 | | | VIISRDDSKSSVYLQMNSLRAEDTAVYYC |
| | | | | TRRWNNEWGQGTTVTVSS |
| J415-1 | | | | |
| V_{H} | Artificial-deimmunized | | 55 | ggatcctataaatctctggccatgaagtctgggagctgaggatgtc |
| Deimmunized | | | | tgtctgcatcagcccgggctcccaatgaccctttctgactcccaag |
| (complimentary | heavy chain | | | gtgtccctagtccttcatgacctgaaattcagatacacacatttcccc |
| strand of | J415-1 | | | cccaacaaatgcagtaaaatctatttaagctgaatagaagagagag |
| SEQ ID NO:53) | | | | gttgtaaggactcacctgaggagactgtgacagtggtgccttggc |
| | | | | cccagaaattattccatcgcctggtacagtaataaacggcagtgtct |
| J415-1 | | | | tcagctctcaaactgttcatttgcaggtagacactactcttggaatca |
| | | | | tctcttgagatgatgaccctccctttcacagactccgcataatgtgtt |
| | | | | gcaaaattattagattgcgatctaataagagcaacccactcaagcc |
| | | | | ccttctctggagtctggcggacccagttcatccagtaattactgaaa |
| | | | | gtgaatccagaggctttacaggagattttcatggaccctccaggttg |
| | | | | caccaagcctcctccagactcctcaagtttcacttcggagtggaca |
| | | | | cctgtggagagaaaggcaaagtggatgtcattgtcacccatatata |
| | | | | tgtccagacctcaagcctgctactgtgagccccttacctgtagctgt |
| | | | | tgctaccaagaagaggatgatacagctccatcccatggtgaggtc |
| | | | | ctgtgtgctcagtaactgtagagagaactgtgatctcatgtttttctgt |
| | | | | ttgtggtatagacaaacctatatttaccatgtagattcagaggatttg |
| | | | | catattcataagctt |
| V_{L} | Artificial-deimmunized | | 56 | aagcttatgaatatgcaaatcctctgaatctacatggtaaatataggt |
| Deimmunized | | | | ttgtctataccacaaacagaaaaacatgagatcacagttctctctac |
| J415-1 | light chain | | | agttactgagcacacaggacctcaccatgggatggagctgtatca |
| | J415-1 | | | tcctcttcttggtagcaacagctacaggtaaggggctcacagtagc |
| CDS (122-160) | | | | aggcttgaggtctggacatatatatgggtgacaatgacatccacttt |
| | | | | gcctttctctccacaggtgtccactccaacattgtaatgacccaatc |
| & | | | | ccccaaatccatgtccgcctcagcaggagagaggatgaccttgac |
| CDS (249-581) | | | | ctgcaaggccagtgagaattccggtacttatgtgtcctggtatcaac |
| | | | | agaaaccaacacagtctcctaagatgttgatatacggggcatccaa |
| | | | | ccggttcactggggtcccagatcgcttctccggcagtggatctgga |
| | | | | acagatttcattctgaccgccagcagtgtgcaggcagaagaccct |
| | | | | gtagattattactgtggacagagttacacctttccgtacacgttcgga |
| | | | | ggggggaccaagctggaaatgaagcgtgagtagaatttaaacttt |
| | | | | gcttcctcagttggatcc |
| V_{L} | Artificial-deimmunized | | 57 | MGWSGI1ILFLVATGVHSNIVMTQSPKSMS |
| Deimmunized | | | | ASAGERMTLTCKASENSGTYVSWYQQKP |
| (predicted a.a. | light chain | | | TQSPKML1YGASNRFTGVPDRFSGSGSGTD |
| of SEQ ID NO:56) | J415-1 | | | FILTASSVQAEDPVDYYGGQSYTFPYTFGG |
| | | | | GTKLEMK |
| J415-1 | | | | |
| V_{L} | Artificial-deimmunized | | 58 | ggatccaactgaggaagcaaagtttaaattctactcacgcttcatttc |
| Deimmunized | | | | cagcttggtcccccctccgaacgtgtacggaaaggtgtaactctgt |
| (complimentary | light chain | | | ccacagtaataatctacagggtcttctgcctgcacactgctggcgg |
| strand of | J415-1 | | | tcagaatgaaatctgttccagatccactgccggagaagcgatctgg |
| SEQ ID NO:56) | | | | gaccccagtgaaccggttggatgccccgtatatcaacatcttagga |
| | | | | gactgtgttggtttctgttgataccaggacacataagtaccggaatt |
| J415-1 | | | | ctcactggccttgcaggtcaaggtcatcctctctcctgctgaggcg |
| | | | | gacatggatttgggggattgggtcattacaatgttggagtggacac |
| | | | | ctgtggagagaaaggcaaagtggatgtcattgtcacccatatatat |
| | | | | gtccagacctcaagcctgctactgtgagccccttacctgtagctgtt |
| | | | | gctaccaagaagaggatgatacagctccatcccatggtgaggtcc |
| | | | | tgtgtgctcagtaactgtagagagaactgtgatctcatgtttttctgttt |
| | | | | gtggtatagacaaacctatatttaccatgtagattcagaggatttgca |
| | | | | tattcataagctt |
| V_{H} | Artificial-deimmunized | | 59 | EVKLEESGGGLVQPGGSMKISGVASGFTFS |
| Deimmunized | | | | NYWMNWVRQTPEKGLEWVALIRSQSNNF |
| J415-2 | heavy chain | | | ATHYAESVKGRVHSRDDSKSSVYLQMNS |
| J415-2 | | | | LRAEDTAVYYCTRRWNNFWNNFWGQGTTVTVS |
| | | | | S |
| V_{H} | Artificial-deimmunized | | 60 | EVKLEESGGGLVQPGGSMKISCVASGFTFS |
| Deimmunized | | | | NYWMNWVRQTPEKGLEWVAEIRSQSNNF |
| 3415-3 | heavy chain | | | ATHYAESVKGRVIISRDDSKSSVYLQMI4S |
| | J415-3 | | | LRAEDTAVYYCTRRWNNFWGQGTITVTVS |
| | | | | S |
| J415 V_{H} (DI) | Artificial-deimmunized | | 61 | EVKLEESGGGLVQPGGSMKISCVASGFTFS |
| majority | majority | | | NYWMNWVRQTPEKGLEWVAELRSQSNNF |
| sequence | sequence | | | ATHYAESVKGRVHSRDDSKSSVYLQMNS |
| | | | | LRAEDTAVYYCTRRWNNFWGQGTFI7VTVS |
| | | | | S |
| V_{L} | Artificial-deimmunized | | 62 | NIVMTQSPKSMSASAGERMTLTCKASENV |
| Deimmunized | | | | GTYVSWYQQKPTQSPKMLIYGASNRFTGV |
| J415-2 | light chain | | | PDRFSGSGSGTDFILTASSVQAEDPVDYYC |
| | J415-2 | | | GQSYTFPYTFGGGTKLEMK |
| V_{L} | Artificial-deimmunized | | 63 | N1VMTQSPKSMSASAGERMTLTCKASENV |
| Deimmunized | | | | GTYVSWYQQKPTQSPKMLIYGASNRFTGV |
| J415-3 | light chain | | | PDRFSGSGSGTDFILTASSVQAEDLVDYYC |
| | J415-3 | | | GQSYTFPYTFGGGTKLEMK |
| V_{L} | Artificial-deimmunized | | 64 | NIVMTQSPKSMSASAGERMTLTCKASENV |
| Deimmunized | | | | GTYVSWYQQKPTQSPKMLIYGASNRFTGV |
| J415-4 | light chain | | | PDRFSGSGSGTDFILTISSVQAEDLVDYYC |
| | J415-4 | | | GQSYTFPYTFGGGTKLEMK |
| V_{L} | Artificial-deimmunized | | 65 | N1VMTQFPKSMSASAGERMTLTCKASENV |
| Deimmunized | | | | GTYVSWYQQKPEQSPKMLIYGASNRFTGV |
| J415-6 | light chain | | | PDRFSGSGSGTDFILTISSVQAEDLVDYYC |
| | J415-6 | | | GQSYTFPYTFGGGTKLEMK |
| V_{L} | Artificial-deimmunized | | 66 | N1VMTQFPKSMSASAGERVTLTCKASENV |
| Deimmunized | | | | GTYVSWYQQKPTQSPKMLIYGASNRFTGV |
| J415-7 | light chain | | | PDRFSGSGSGTDHLTISSVQAEDLVDYYC |
| | J415-7 | | | GQSYTFPYTFGGGTKLEMK |
| V_{L} | Artificial-deimmunized | | 67 | NIVMTQFPKSMSASAGERMTLTCKASENS |
| Deimmunized | | | | GTYVSWYQQKIPEQSPKML1YGASNRFTGV |
| 3415-8 | light chain | | | PDRFSGSGSGTDFILTISSVQAEDLVDYYC |
| | J415-8 | | | GQSYTFPYTFGGGTKLEMK |
| J415 V_{L} (DI) | Artificial-deimmunized | | 68 | N1VMTQFPKSMSASAGERMTLTCKASENV |
| majority | majority | | | GTYVSWYQQKPTQSPKMLIYGASNRFTGV |
| sequence | sequence | | | PDRFSGSGSGTDFLLTISSVQAEDLVDYYC |
| | | | | GQSYTFPYTFGGGTKLEMK |
| MuV_{H}IIIC | Mus musculus | | 69 | EVKLEESGGGLVQPGGSMKLSCVASGFTF |
| | | | | SNYWMNWVRQSPEKGLEWVAEIRLKSDN |
| | | | | YATHYAESVKGRFTISRDDSKSSVYLQMN |
| | | | | NLRAEDTGIYYCTTGGYGGRRSWFAYWG |
| | | | | QGTLVTVSS |
| J415 V_{H}/MuV | Artificial-majority | | 70 | EVKLEESGGGLVQPGGSMIKLSCVASGETF |
| _{H}IIIC | sequence | | | SNYWMNWVRQSPEKGLEWVAEIRLQSDN |
| majority | | | | FATHYAESVKGRVHSRDDSKSSVYLQMN |
| sequence | | | | NLRAEDTGIYYCTTGGYGGRRSWNAFWG |
| | | | | QGTLVTVSS |
| Mu_{V}L1 | Mus musculus | | 71 | DIVMTQSPSSLAVSAGEKVTMSCKSSQSLL |
| | | | | NSGNQKNYLAWYQQKPGQSPKLLIYWAS |
| | | | | TRESGVPDRFTGSGSGTDFTLTISSVQAED |
| | | | | LAVYYCQNDYSYPLTFGAGTKLELK |
| J415V_{L}/MuV | Artificial-majority | | 72 | DIVMTQSPSSLAVSAGEKVTLSCKASESLL |
| _{L}1 majority | sequence | | | NVGNQKTYVAWYQQKPGQSPKLLIYGAS |
| sequence | | | | TRESGVPDRFTGSGSGTDFILTISSVQAEDL |
| | | | | AVYYCGNSYSFPLTFGGGTKLELK |
| J533 V_{H} | Mus musculus | | 73 | gaggtccagctgcagcagtctggacctgagctggttaagcctggg |
| | | | | gcttcagtgaagatgtcctgcaaggcttctggatacacattcactgg |
| CDS (1-354) | | | | ctatgttatgcactgggtgaagcagaagcctggacaggtccttgag |
| | | | | tggattggatatattaatccttacaatgatgttactaggtataatggga |
| | | | | agttcaaaggcaaggccacactgacctcagacaaatattccagca |
| | | | | cagcctacatggagctcagcggcctgacctctgaggactctgcgg |
| | | | | tctattactgtgcaagaggggagaactggtactactttgactcctgg |
| | | | | ggccgaggcgccactctcacagtctcctca |
| J533 V_{H} | Mus musculus | | 74 | EVQLQQSGPELVKPGASVKMSCKASGYTF |
| (predicted | | | | TGYVMHWVKQKPGQVLEWIGYINPYNDV |
| amino acid of | | | | TRYNGKFKGKATLTSDKYSSTAYMELSGL |
| SEQ ID NO:73) | | | | TSEDSAVYYCARGENWYYFDSWGRGATL |
| | | | | TVSS |
| J533 V_{H} | Mus musculus | | 75 | tgaggagactgtgagagtggcgcctcggccccaggagtcaaagt |
| (complementary | | | | agtaccagttctcccctcttgcacagtaatagaccgcagagtcctc |
| strand of | | | | agaggtcaggccgctgagctccatgtaggctgtgctggaatatttg |
| SEQ ID NO:73) | | | | tctgaggtcagtgtggccttgcctttgaacttcccattatacctagta |
| | | | | acatcattgtaaggattaatatatccaatccactcaaggacctgtcc |
| | | | | aggcttctgcttcacccagtgcataacatagccagtgaatgtgtatc |
| | | | | cagaagccttgcaggacatcttcactgaagccccaggcttaacca |
| | | | | gctcaggtccagactgctgcagctggacctc |
| J533 V_{L} | Mus musculus | FIG. | 76 | gacattgtgctgacccaatctccagcttctttggctgtgtctctagga |
| | | 10A | | cagagggccaccatatcctgcagagccagtgaaagtattgatagtt |
| CDS (1-333) | | | | atgacaatacttttatgcactggtaccagcagaaaccaggacagcc |
| | | | | acccaacctcctcatctttcgtgcatccatcctagaatctgggatcc |
| | | | | ctgccaggttcagtggcagtgggtctgggacagacttcaccctca |
| | | | | ccatttatcctgtggaggctgatgatgttgcaacctattactgtcacc |
| | | | | aaagtattgaggatccgtacacgttcggaggggggaccaagctg |
| | | | | gaaataaaa |
| J533 V_{L} | Mus musculus | FIG. | 77 | DIVLTQSPASLAVSLGQRATISCRASESIDS |
| (predicted | | 10A | | YDNTFMHWYQQKPGQPPNLLLFRASILES |
| amino acid of | | | | GIPARFSGSGSGTDFTLTIYPVEADDVATY |
| SEQ ID NO:76) | | | | YCHQSLEDPYTFGGGTKLEIK |
| J533 V_{L} | Mus musculus | FIG. | 78 | ttttatttccagcttggtcccccctccgaacgtgtacggatcctcaat |
| (complementar | | 10A | | actttggtgacagtaataggttgcaacatcatcagcctccacaggat |
| y strand of | | | | aaatggtgagggtgaagtctgtcccagacccactgccactgaacc |
| SEQ ID NO:76) | | | | tggcagggatcccagattctaggatggatgcacgaaagatgagg |
| | | | | aggttgggtggctgtcctggtttctgctggtaccagtgcataaaagt |
| | | | | attgtcataactatcaatactttcactggctctgcaggatatggtggc |
| | | | | cctctgtcctagagacacagccaaagaagctggagattgggtcag |
| | | | | cacaatgtc |
| MuV_{H}II | Mus musculus | | 79 | EVQLQQSGPELVKPGASVKISCKASGyTFT |
| | | | | DYYMNNWVKQSPGKSLEWIGDINPGNGG |
| | | | | TSYNQKFKGKATLTVDKSSSTAYMQLSSL |
| | | | | TSEDSAVYYCARGYYSSSYMAYYAFDYW |
| | | | | GQGTTVTVSS |
| J533V_{H}/MuV | Artificial-majority | | 80 | EVQLQQSGPELVKPGASVKISCKASGYTFT |
| majority | sequence | | | GYVMNNWVKQSPGQVLEWIGDINPGNGG |
| sequence | | | | TSYNGKFKGKATLTVDKSSSTAYMELSGL |
| | | | | TSEDSAVYYCARGENSSSYMAYYAFDSW |
| | | | | GQGATVTVSS |
| MuV_{L}-3 | Mus musculus | | 81 | D1VLTQSPASLAVSLGQRATISCRASESVDS |
| | | | | YGNSFMHWYQQKPGQPPKLL1YAASNLES |
| | | | | GVPARFSGSGSGTDFTLNIIIPVEEDDAATY |
| | | | | YCQQSNEDPPWTFGGGTKLEIK |
| J533V_{L}/MuV_{L}-3 | Artificial-marjority | FIG. | 82 | DIVLTQSPASLAVSLGQRATISCRASESVDS |
| majority | sequence | 10B | | YGNSFMHWYQQKiPGQPPNLLLFAASILES |
| sequence | | | | GVPARFSGSGSGTDFTLTLHPVEADDAATY |
| | | | | YCQQSIEDPPYTFGGGTKLEIK |
| E99 V_{H} | Mus musculus | FIG. | 83 | caggtgcagctaaaggagtcaggacctggcctggtggcgtcctc |
| | | 11A | | acagagcctgtccatcacatgcaccgtctcaggattctcattaacc |
| CDS (1-363) | | | | gcctatggtattaactgggttcgccagcctccaggaaagggtctgg |
| | | | | agtggctgggagtgatatggcctgatggaaacacagactataattc |
| | | | | aactctcaaatccagactgaacatcttcaaggacaactccaagaac |
| | | | | caagttttcttaaaaatgagcagtttccaaactgatgacacagccag |
| | | | | atacttctgtgccagagattcgtatggtaacttcaagaggggttggtt |
| | | | | tgacttctggggccagggcaccactctcacagtctcctca |
| E99 V_{H} | Mus musculus | FIG. | 84 | QVQLKESGPGLVASSQSLSITCTVSGFSLT |
| (predicted | | 11A | | AYGLNWVRQPPGKGLEWLGVIWPDGNTD |
| amino acid of | | | | YNSTLKSRLNLFKDNSKNQVFLKMSSFQT |
| SEQ ID NO:83) | | | | DDTARYFCARDSYGNFKRGWFDFWGQGT |
| | | | | TLTVSS |
| E99 V_{H} | Mus musculus | FIG. | 85 | tgaggagactgtgagagtggtgccctggccccagaagtcaaacc |
| (complementary | | 11A | | aacccctcttgaagttaccatacgaatctctggcacagaagtatctg |
| strand of | | | | gctgtgtcatcagtttggaaactgctcatttttaagaaaacttggttctt |
| SEQ ID NO:83) | | | | ggagttgtccttgaagatgttcagtctggatttgagagttgaattata |
| | | | | gtctgtgtttccatcaggccatatcactcccagccactccagaccct |
| | | | | ttcctggaggctggcgaacccagttaataccataggcggttaatga |
| | | | | gaatcctgagacggtgcatgtgatggacaggctctgtgaggacgc |
| | | | | caccaggccaggtcctgactcctttagctgcacctg |
| E99 V_{L} | Mus musculus | FIG. | 86 | aacattgtgatgacccagtctcaaaaattcatgtccacatcaccagg |
| CDS (1-321) | | 12A | | agacagggtcagggtcacctgcaaggccagtcagaatgtgggtt |
| | | | | ctgatgtagcctggtatcaagcgaaaccaggacaatctcctagaat |
| | | | | actgatttactcgacatcctaccgttacagtggggtccctgatcgctt |
| | | | | cacagcctatggatctgggacagatttcactctcaccattaccaatg |
| | | | | tgcagtctgaagacttgacagagtatttctgtcagcaatataatagct |
| | | | | atcctctcacgttcggtgctgggaccaagctggagctgaaa |
| E99 V_{L} | Mus musculus | FIG. | 87 | N1VMTQSQKFMSTSPGDRVRVTCKASQNV |
| (predicted | | 12A | | GSDVAWYQAKPGQSPRILIYSTSYRYSGVP |
| amino acid of | | | | DRFTAYGSGTDFTLTITNVQSEDLTEYFCQ |
| SEQ ID NO:86) | | | | QYNSYPLTFGAGTKLELK |
| E99 V_{L} | Mus musculus | FIG. | 88 | tttcagctccagcttggtcccagcaccgaacgtgagaggatagcta |
| (complementary | | 12A | | ttatattgctgacagaaatactctgtcaagtcttcagactgcacattg |
| strand of | | | | gtaatggtgagagtgaaatctgtcccagatccataggctgtgaagc |
| SEQ ID NO:86) | | | | gatcagggaccccactgtaacggtaggatgtcgagtaaatcagta |
| | | | | ttctaggagattgtcctggtttcgcttgataccaggctacatcagaac |
| | | | | ccacattctgactggccttgcaggtgaccctgaccctgtctcctggt |
| | | | | gatgtggacatgaatttttgagactgggtcatcacaatgtt |
| MuV_{H}IB | Mus musculus | FIG. | 89 | QVQLKESGPGLVASSQSLSITGTVSGFSLT |
| | | 11B | | AYGINWVRQPPGKGLEWLGV1WPDGNTD |
| | | | | YNSTLKSRLNIFKDNSKNQVFLKMSSFQT |
| | | | | DDTARYFCARDSYGNFKRGWFDFWGQGT |
| | | | | TLTVSS |
| E99 V_{H}/MuV_{H} | Artificial-marjority | FIG. | 90 | QVQLKESGPGLVASSQSLSITCTVSGFSLT |
| 1B | sequence | 11B | | AYGINWVRQPPGKGLEWLGVIWPDGNTD |
| majority | | | | YNSTLKSRLNIFKDNSKNQVFLKMSSFQT |
| sequence | | | | DDTARYFCARDSYGNFKRGWFDFWGQGT |
| | | | | TLTVSS |
| MuV_{L}-1 | Mus musculus | FIG. | 91 | D1VMTQSPSSLAVSAGEKVTMSCKSSQSLL |
| | | 12B | | NSGNQKNYLAWYQQKPGQSPKLLIYWAS |
| | | | | TRESGVPDRFTGSGSGTDFTLTISSVQAED |
| | | | | LAVYYGQNDYSYPLTFGAGTKLELK |
| E99 V_{L}/MuV_{L} | Artificial-majority | FIG. | 92 | DIVMTQSQSSLAVSAGDKVTVSCKASQSL |
| -1 | sequence | 12B | | LNVGSDKNYVAWYQAKIPGQSPKLLIYSAS |
| majority | | | | TRESGVPDRFTGSGSGTDFTLTISSVQAED |
| sequence | | | | LAVYFCQNDNSYPLTFGAGTKLELKRA |

Humanized or CDR-grafted antibody molecules or immunoglobulins can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference.

Winter describes a CDR-grafting method that may be used to prepare the humanized antibodies of the present invention (UK Patent Application GB 2188638A, filed on Mar. 26, 1987; Winter U.S. Pat. No. 5,225,539), the contents of which are expressly incorporated by reference. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above. The recombinant DNA encoding the humanized antibody, or fragment thereof, can then be cloned into an appropriate expression vector.

Also within the scope of the invention are humanized antibodies in which specific amino acids have been substituted, deleted or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the contents of which are hereby incorporated by reference. The acceptor framework can be a mature human antibody framework sequence or a consensus sequence. As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

Other techniques for humanizing immunoglobulin chains, including antibodies, are described in Padlan et al. EP 519596 A1, published on Dec. 23, 1992.

The anti-PSMA antibody, or antigen fragment thereof, may also be modified by specific deletion of human T cell epitopes or “deimmunization” by the methods disclosed in WO 98/52976 and WO 00/34317, the contents of which are specifically incorporated by reference herein. Briefly, the murine heavy and light chain variable regions of an anti-PSMA antibody can be analyzed for peptides which bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the murine V_{H} and V_{L} sequences, as described in WO 98/52976 and WO 00/34317. These motifs bind to any of the 18 major MHC class II DR allotypes, and thus, constitute potential T cell epitopes. Potential T-cell epitopes detected can be eliminated by substituting small numbers of amino acid residues in the variable regions, or preferably, by single amino acid substitutions. As far as possible conservative substitutions are made, often but not exclusively, an amino acid common at this position in human germline antibody sequences may be used. Human germline sequences are disclosed in Tomlinson, I. A. et al. (1992) J. Mol. Biol. 227:776-798; Cook, G. P. et al. (1995) Immunol. Today Vol. 16 (5): 237-242; Chothia, D. et al. (1992) J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). After the deimmunized V_{H} and V_{L} of an anti-PSMA antibody are constructed by mutagenesis of the murine V_{H} and V_{L} genes. The mutagenized variable sequence can, optionally, be fused to a human constant region, e.g., human IgG1 or κ constant regions.

In some cases a potential T cell epitope will include residues which are known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs were eliminated by substitutions outside the CDRs. In some cases, an alteration within a CDR is the only option, and thus variants with and without this substitution should be tested. In other cases, the substitution required to remove a potential T cell epitope is at a residue position within the framework that might be critical for antibody binding. In these cases, variants with and without this substitution should be tested. Thus, in some cases several variant deimmunized heavy and light chain variable regions were designed and various heavy/light chain combinations tested in order to identify the optimal deimmunized antibody. The choice of the final deimmunized antibody can then be made by considering the binding affinity of the different variants in conjunction with the extent of deimmunization, i.e., the number of potential T cell epitopes remaining in the variable region.

The recombinant deimmunized antibody can be transfected into a suitable host cell for expression, for example, NS0 or CHO cells, to produce complete recombinant antibodies.

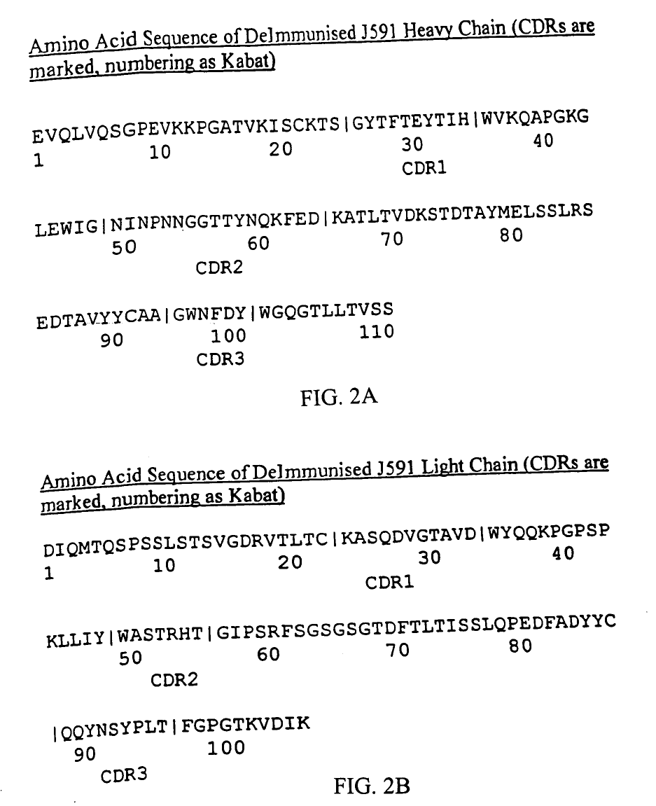

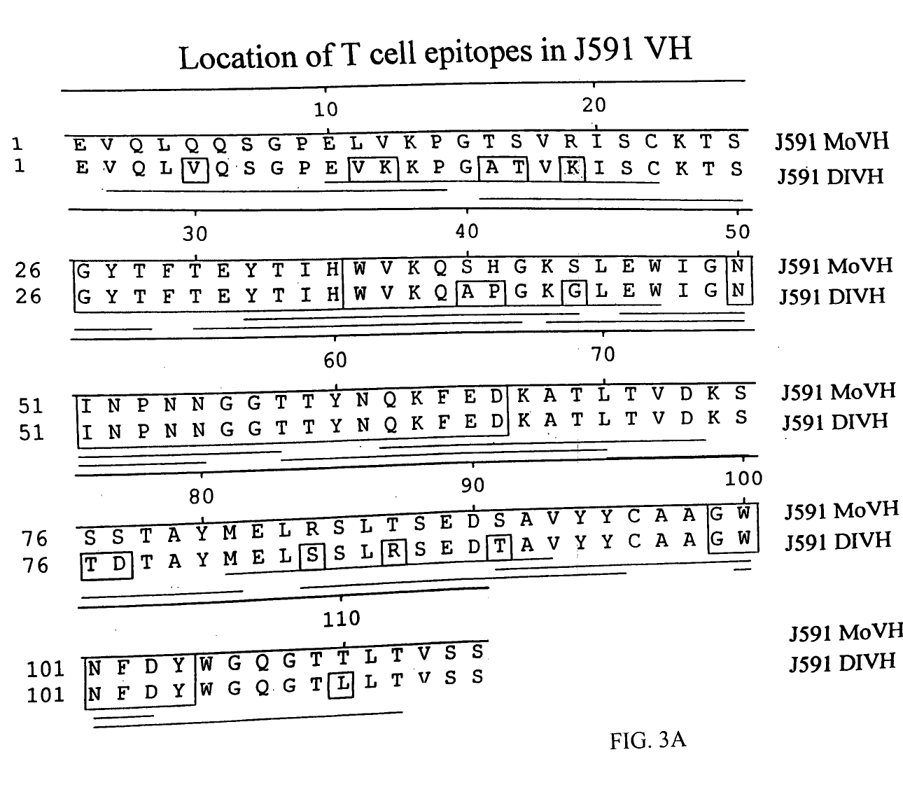

In one embodiment, deimmunized V_{H} and V_{L} of murine J591 regions were constructed by mutagenesis of the murine V_{H} and V_{L} genes. The murine J591 variable region sequences are shown in FIGS. 1A-1B. Potential epitopes (identified using a peptide threading program) in murine J591 heavy chain and light chain variable regions are shown in FIGS. 2A and 2B, respectively. The 13-mer peptides predicted to bind to MHC class II are indicated by the underline, the CDRs are located at residues 26 to 35, 50 to 66, and 99 to 104 of FIG. 2A and residues 24 to 34, 50 to 56, and 89 to 97 of FIG. 2B, and residues altered in the deimmunized heavy and light chain variable regions are boxed. Where possible, amino acid substitutions are those commonly used in human germline heavy and light chain variable regions. In addition to the in silico analysis using the peptide threading software, a database of human MHC class II binding peptides was searched for motifs present in the murine J591 sequence.

The following 13-mers (labeled by first linear residue number of the 13-mer) of the murine J591 heavy chain variable region were predicted to bind to MHC Class II were 2, 10, 16, 30, 32, 35, 43, 46, 58, 62, 70, 81, 84, 91, and 100 (FIG. 2A). An explanation of the rationale behind changes made to the residues in the murine J591 heavy chain variable region is set forth below (note residues altered are identified under the Kabat numbering system):

5Q→V removes the potential epitope at residue 2;

11,12LV→VK remove the potential epitope at residue 10;

12V→K is also changed to remove a motif from the database of human MHC class II binding peptides;

16,17TS→AT, and 19R→K remove the potential epitope at residue 16;

the epitope at residue 30 spans CDR1 and is therefore unaltered;

40,41SH→AP removes potential epitopes at residues 32 and 35;

44S→G reduces binding score for epitope at 43, this 13 mer spans CDR2;

the epitopes at residues 46, 58 and 62 span CDR2, and are thus unaltered;

75,76SS→TD remove the potential epitope at residue 70;

82aR→S, 83T→R remove potential epitopes at residues 81 and 84;

87S→T this change made to remove a motif from the database of human MHC class II binding peptides;

the epitope at residue 91 spans CDR3 and is therefore unaltered; and

108T→L removes the potential epitope at residue 100.

The following 13-mers (labeled by first linear residue number of the 13-mer) of the murine J591 light chain variable region that were predicted to bind to MHC Class II molecules were 1, 8, 17, 27, 30, 31, 35, 45, 47, 56, 60, 71, 73, 81, 94 (FIG. 2B). An explanation of the rationale behind changes made to the residues in the murine J591 light chain variable region is set forth below (note residues altered are identified under the Kabat numbering system):

3V→Q removes potential epitope at residue 1;

8-11HKFM→PSSL removes potential epitope at residue 8(13);

20-22SII→TLT removes potential epitopes at residues 17 and 20;

21I→L is also changed to remove a motif from the database of human MHC class II binding peptides;

the epitope at residue 27 spans CDR1 and is therefore unaltered;

42Q→P reduces the binding score for the epitope at residue 31;

the epitopes at residues 44 and 47 span CDR2 and are thus unaltered;

58V→I is changed to remove a motif from the database of human MHC class II binding peptides;

60D→S, 62T→S removes the epitopes at residues 56 and 60;

76-78TNV→SSL, 80S→P, 83L→F removes the epitopes at residues 71, 73, 76, and 81;

87F→Y I is changed to remove a motif from the database of human MHC class II binding peptides;

100 A→P and 103 M→K remove the epitope at residue 94; and

104 L→V and 106 L→I are changed to remove a motif from the database of human MHC class II binding peptides.

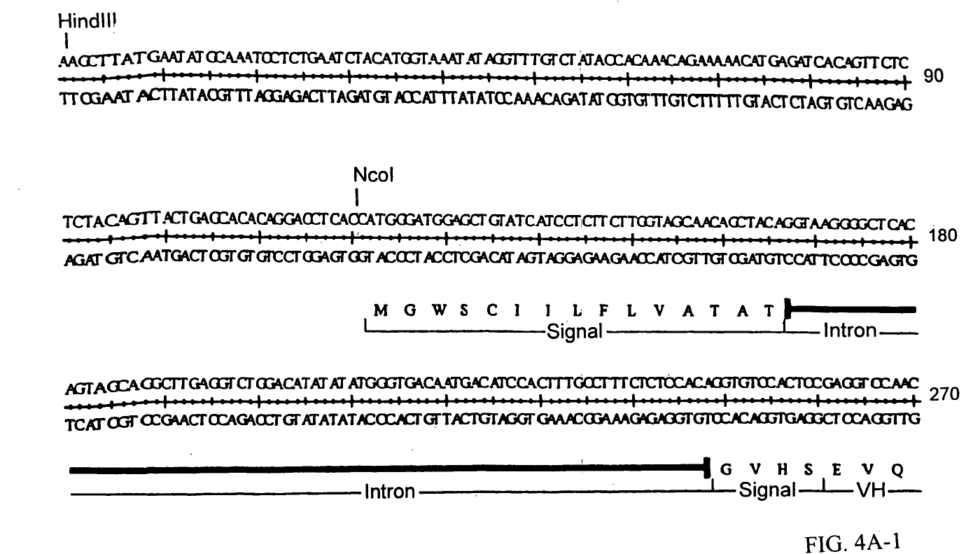

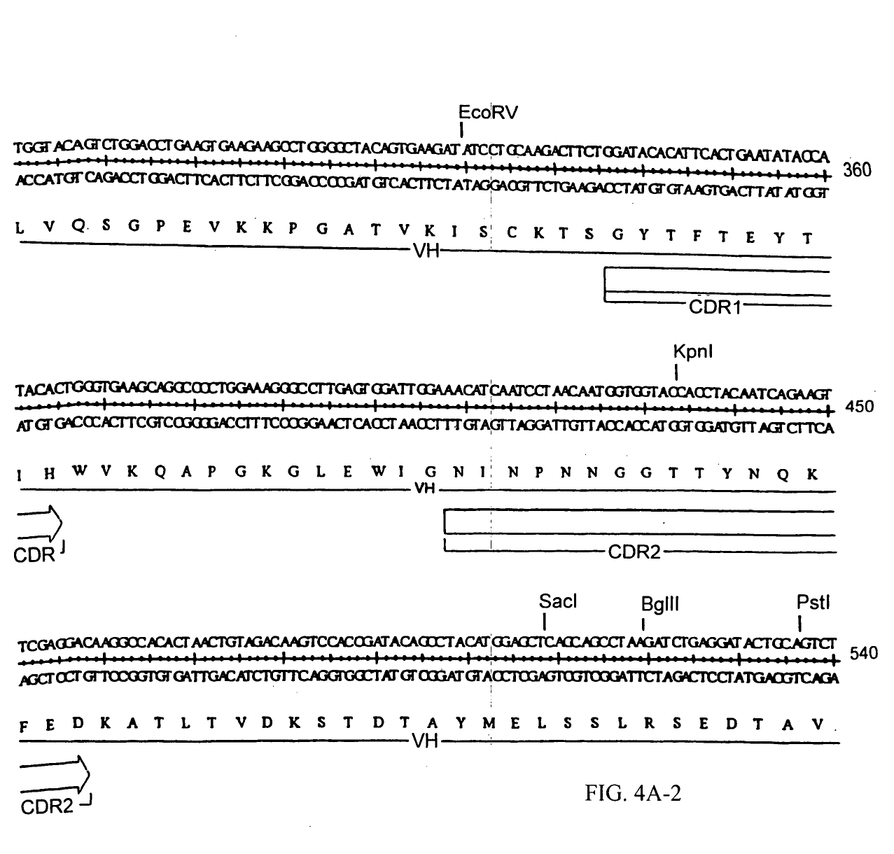

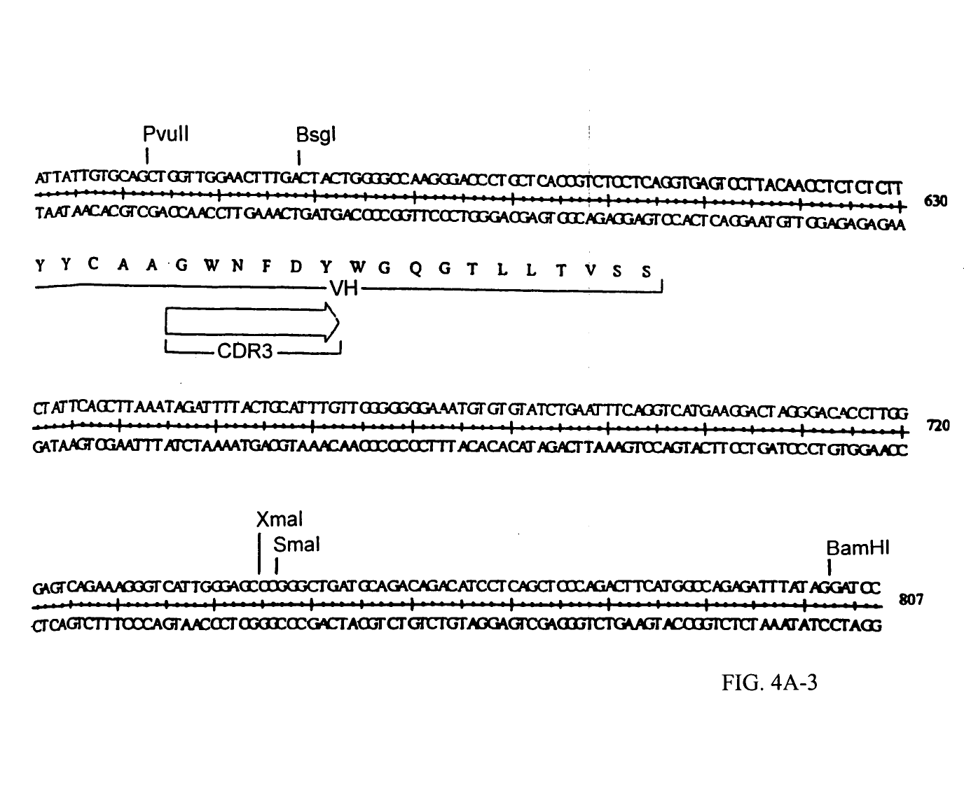

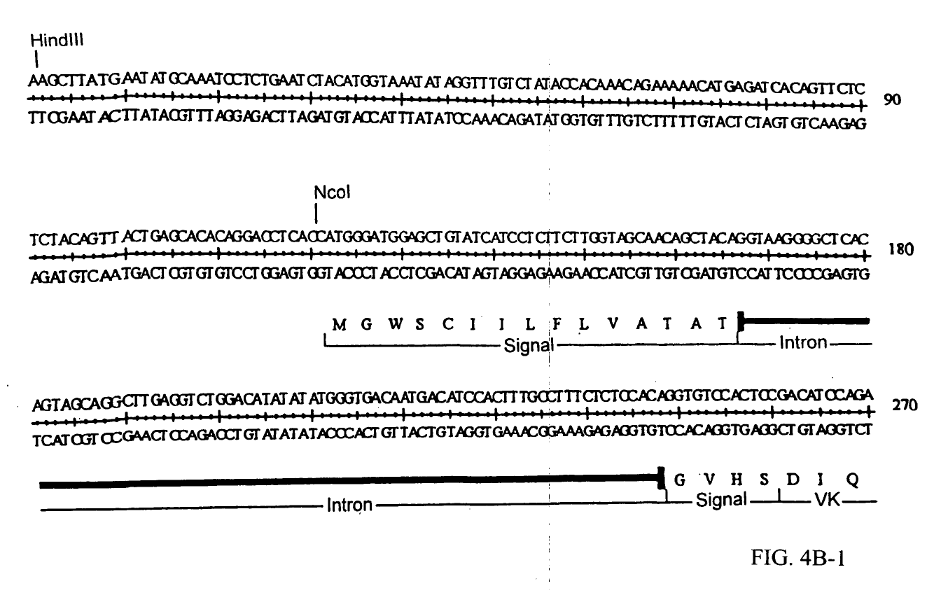

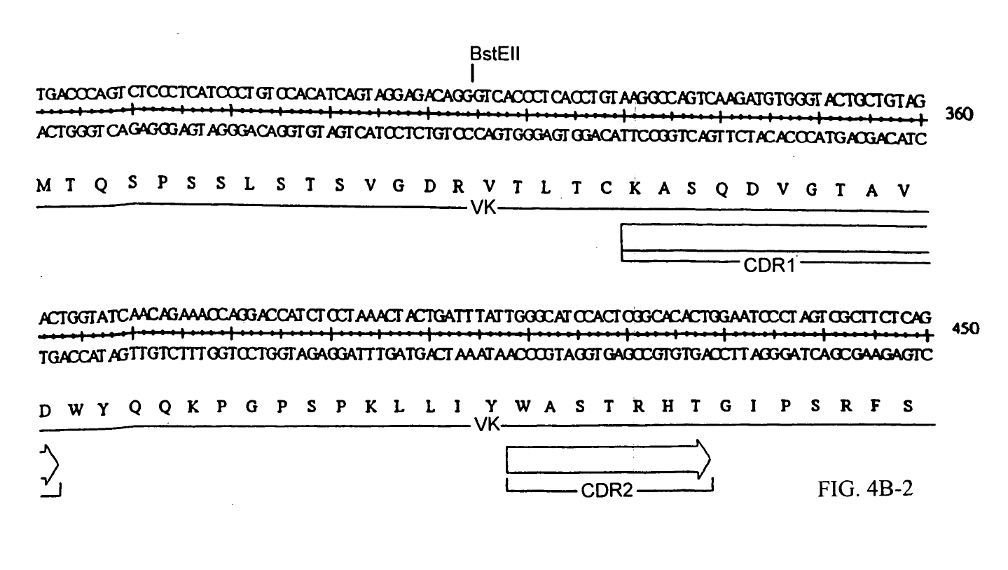

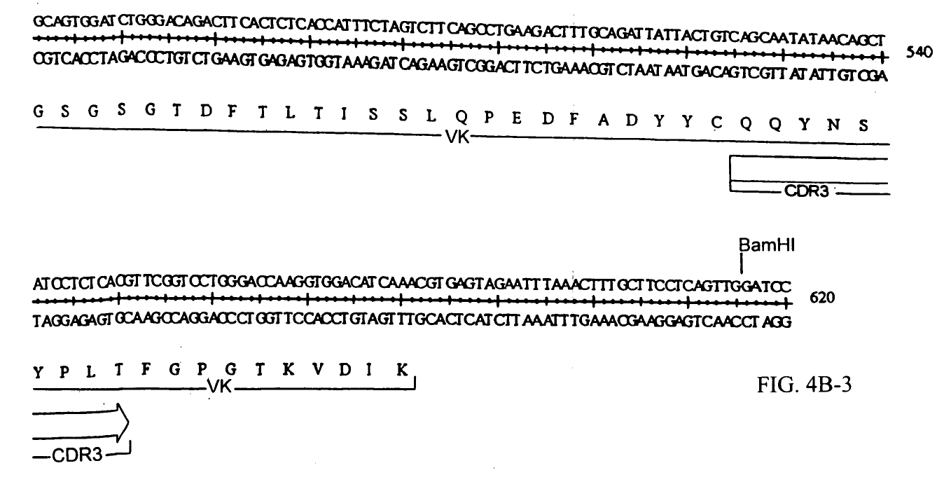

The amino acid and nucleotide sequences for the deimmunized J591 heavy and light chain variable regions are shown in FIGS. 2A-2B and 4A-4B, respectively (see also Table 1).

Human IgG1 or κ constant regions were added and the composite genes transfected into NS0 cells to produce complete recombinant anti-PSMA antibodies. These antibodies bound to PSMA (on LNCap cells) as efficiently as the original murine antibody, and have reduced or no immunogenicity in man.

The design of deimmunized J415 was similar to the making of the deimmunized J591 antibody. The heavy and light chain sequences were cloned from the hybridoma designated HB-12109. These sequences were cloned, sequenced and expressed as a chimeric antibody for use as a control antibody. The murine V region sequences were subjected to peptide threading to identify potential T cell epitopes, through analysis of binding to 18 different human MHC class II allotypes. The results of the peptide threading analysis for the murine sequences are shown in Table 2.

TABLE 2
Potential T cell epitopes in murine J415 sequences

| | Number of | Location of potential epitopes+ (no. of |
|---|---|---|
| | potential | potential MHC binders from 18 groups |
| Sequence | T cell | tested) |
| Murine | 12 | 10(17), 16(13), 21(9), 30(6), 35(16), 43(8), |
| J415 V_{H} | | 46(6), 49(8), 64(6), 80(15), 86(15), 104(6) |
| Murine | 13 | 5(5), 11(18), 13(11), 17(5), 27(8), 31(7), |
| J415 V_{K} | | 56(15), 60(12), 70(5), 71(11), 73(17), 76(7), |
| | | 81(17) |

+first amino acid of potential epitope, numbering E or N amino acid number1 to S or K amino acid number 107 and 116 for V_{H} and V_{K} respectively.

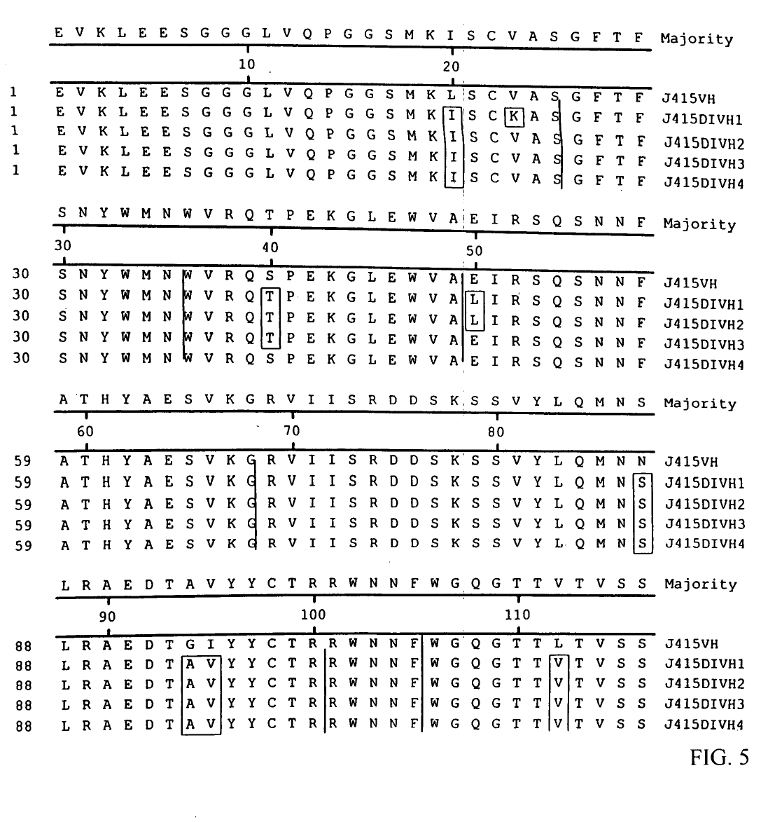

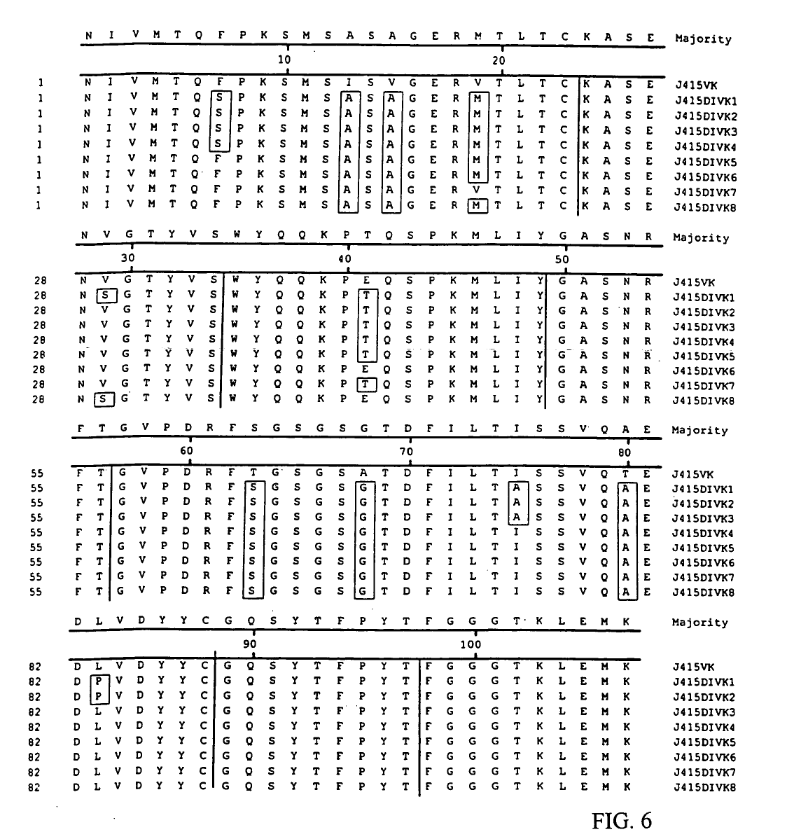

Primary deimmunized V_{H} and V_{L} sequences were defined (J415DIVH1, J415DIVK1). As generation of the primary deimmunized sequences requires a small number of amino acid substitutions that might affect the binding of the final deimmunized molecule, three other variant V_{H}S and seven other V_{L}S were designed (see FIGS. 5 and 6). The nucleotide sequences for the primary deimmunized V_{H} and V_{L} regions are shown in FIGS. 7A and 8A, respectively. Comparisons of the amino acid sequences of the murine and deimmunized V regions of J415 are shown in FIG. 5 for V_{H} and FIG. 6 for V_{L}.

An explanation of the rational behind some of the changes made to the residues in the murine J415 heavy chain variable region is set forth below (note residues altered are identified according to the linear numbering shown in FIG. 5):

20L→I removes the potential epitope at residues 10 and 16;

87N→S removes the potential epitopes at residues 80 and 86;

94,95GI→AV remove the potential epitope at residue 86; and

112L→V removes the potential epitope at residue 104.

Changes to Residues of Murine J415

An explanation of the rational behind some of the changes made to the residues in the murine J415 light chain variable region is set forth below (note residues altered are identified according to the linear numbering shown in FIG. 6):

13I A removes the potential epitopes at residues 5, 11 and 13;

15V A removes the potential epitopes at residues 5, 11, and 13;

19V-M removes the potential epitopes at residues 11, 13, and 17;

41E-T removes the potential epitope at residue 31;

63T-S removes the potential epitopes at residues 56 and 60;

68A-G removes the potential epitopes at residues 56 and 60; and

80T-A removes the potential epitopes at residues 70, 71, 73, and 76;

The deimmunized variable regions for J415 were constructed by the method of overlapping PCR recombination. The cloned murine V_{H} and V_{K} genes were used as templates for mutagenesis of the framework regions to the required deimmunized sequences. Sets of mutagenic primer pairs were synthesized encompassing the regions to be altered. The vectors VH-PCR1 and VK-PCR1 (Riechmann et al. (1988) Nature 332:323-7) were used as templates to introduce 5′ flanking sequence including the leader signal peptide, leader intron and the murine immunoglobulin promoter, and 3′ flanking sequence including the splice site, and intron sequences. The deimmunized V regions produced were cloned into pUC19 and the entire DNA sequence was confirmed to be correct for each deimmunized V_{H} and V_{L}.

The deimmunized heavy and light chain V-region genes were excised from pUC 19 as HindIII to BamHI fragments, which include the murine heavy chain immunoglobulin promoter, the leader signal peptide, leader intron, the V_{H} or V_{L} sequence and the splice site. These were transferred to the expression vectors pSVgpt and pSVhyg, which include human IgG1 or κ constant regions respectively and markers for selection in mammalian cells. The DNA sequence was confirmed to be correct for the deimmunized V_{H} and V_{L} in the expression vectors.

For the transfection of expression vectors pSVgptJ415VHHuIgG1 and pSVhygJ415VKHuCK into NS0 (a non-immunoglobulin producing mouse myeloma, obtained from the European Collection of Animal Cell Cultures, Porton UK (ECACC No. 85110505)) cells, 3 and 6 μg of plasmid DNA respectively was prepared and then linearized with PvuI to improve transfection efficiency. The ethanol precipitated DNA was then mixed with a semi-confluent flask of NSO cells that had been harvested by centrifugation and resuspended in 0.5 ml of non-selective Dulbecco's Modified Eagle's Medium (DMEM)(Life Technologies Inc.) in a 0.4 cm gene pulser cuvette. The cells and DNA were chilled on ice for 5 minutes before a pulse of 17OV, 960 μF was applied. The cuvette was returned to ice for a further 20 minutes before being transferred to a 75 cm² flask containing 20 mls non-selective DMEM to recover for 24 hours. The cells were then harvested and resuspended in selective DMEM and plated over 4×96 well plates, 200 μl/well.

To culture NSO cell lines, selection and expansion the cells are grown at 37° C., 5% CO₂ and 10% FBS. To prepare non-selective medium for routine culture of NSO cells, the culture medium is Dulbecco's Modification of Eagle's Medium (DMEM)(Life Technologies, Catalogue No: 31965-023) supplemented with 10% fetal bovine serum of USA origin (Life Technologies, Fetal Bovine Serum Cat No: 16000), Antibiotic/Antimycotic solution (Life Technologies, Cat No: 15240), Gentamycin (Life Technologies, catalogue No: 15710), Sodium pyruvate (Life Technologies, Catalogue No: 11360-039). When growing NSO cells up to saturation for antibody production do not add the xanthine and mycophenolic acid and the FBS is reduced to 5%.

To prepare selective medium for culture of NSO transfectomas, the culture medium is Dulbecco's Modification of Eagle's Medium (DMEM)(Life Technologies, Catalogue No: 31965-023) supplemented with 10% fetal bovine serum of USA origin (Life Technologies, Fetal Bovine Serum Cat No: 16000), Antibiotic/Antimycotic solution (Life Technologies, Cat No: 15240), Gentamycin (Life Technologies, catalogue No: 15710), Sodium pyruvate (Life Technologies, Catalogue No: 11360-039), 250 μg/ml xanthine (Sigma Catalogue No: X-3627, stock made up at 25 mg/ml in 0.5M NaOH), and 0.8 μg/ml mycophenolic acid (Sigma Catalogue No: M-3536, stock made up at 2.5 mg/ml in 100% ethanol).

After approximately 10 days the cell colonies expressing the gpt gene were visible to the naked eye. The plates were then screened for antibody production using the following protocol for human IgG1/κ Screening ELISA. 6 single colonies were picked from wells with high ODs greater than 0.7 into a 24 well cell culture plate. Within 5-6 days the cells were expanded into a 25 cm² flask. The antibody productivity of the selected clones was assayed using the following protocol for human IgG1/κ ELISA from saturated cultures in the 24 well and 25 cm² flasks.

The details of the protocol are as follows. ELISA plates (Dynatech Immulon 2) are coated with 100 μL per well with sheep α human κ antibody (The Binding Site Cat No: AU015) diluted 1:1000 in carbonate/bicarbonate coating buffer pH9.6 (Sigma Cat: C-3041). The coated plate is incubated at 4° C. overnight or 1 hr at 37° C. The plate is then washed 3 times with PBST (PBS with 0.05% Tween 20). The samples are added, 100 μL per well from 24 well plates, 25 μL+75 μL PBST for 96 well plates. Blank wells are treated with PBST only. The reaction mixture is incubated at room temperature for 1 hr. Then, the plate is wash 3 times with PBST (PBS with 0.05% Tween 20). The secondary antibody, peroxidase conjugated sheep α human IgG γ chain specific is added (The Binding Site Cat No: AP004) at a ratio of 1:1000 in PBST, 100 μL per well. The mixture is incubated at room temperature for 1 hour. The mixture is then washed 3 times with PBST (PBS with 0.05% Tween 20).

To make up the substrate, one tablet (20 mg) of OPD (o-PHENYLENE DIAMINE) (Sigma Cat No: P-7288) is dissolved in 45 ml of H₂O plus 5 ml 10× peroxidase buffer (make 10× peroxidase buffer with Sigma phosphate citrate buffer tablets pH 5.0, P-4809), add 10 μL 30%(w/w) hydrogen peroxide (Sigma Cat No: Hi 109) just before use. The substrate is then added at 100 μL per well and incubate RT for 5 min or as required. When the color develops, the reaction can be stopped by adding 25 μL 12.5% H₂SO₄. The results are read at 492 nm.

Expression and Expansion of J415 Deimmunized Antibodies

The clones with the highest productivity were expanded into a 75 cm² flask and then into 2×175 cm² flasks. The cells from one of the 175 cm² flask was used to inoculate 4× triple layer flasks (500 cm², Nalge Nunc International) containing non selective DMEM containing 5% FBS, cells from the other were frozen as detailed in the protocol for freezing NSO cells detailed below.

To cryoprotect mammalian cells and resurrect cells from liquid nitrogen, the following materials are needed: Fetal Bovine serum (Life Technologies Cat No: 16000), DMSO (Sigma, Cat No: D4540), 2 ml cryotubes (Nunc or Greiner), and polystyrene box with walls 1-2 cm thick. Briefly, actively growing cells are harvested by centrifugation (1000 rpm, 5 min) and resuspended at about 10⁷ cells/ml in 10% DMSO/90% FBS. As a rough guide, cells grown to a semi-confluency should be resuspended in 1 ml for a 75 cm² flask or 2.5 ml for a 175 cm² flask. A required number of tubes are cooled and labeled in ice. 1 ml portions are aliquoted to labeled cryotubes. The cryotubes are placed in polystyrene box at −70° C. for at least 4 h, or overnight. The vials are transferred to canes and place in liquid nitrogen. A record of the storage should be made both in the canister index and the central cell line indexing system.

To thaw the cells from liquid nitrogen, the vial is removed from liquid nitrogen and contents are thawed quickly by incubation at 37° C., while swirling in a water bath. The outside of the vial is cleaned with 70% methylated spirits. The suspension is transferred to a universal container. 10 ml of the medium to be used to propagate the cell line is added dropwise, swirling to mix. The cells are harvested by centrifugation (1000 rpm, 5 min). The supernatant is discarded. The cells are resuspended in 20 ml growth medium and transferred to a 75 cm² flask. If low viability is suspected, extra serum can be added to the growth medium to 20%, use only 5 ml, and transfer to a 25 cm² flask.

After 10-14 days the 500 ml to 1 liter static saturated cultures were harvested. Antibody was purified, by ProSepA (Millipore Ltd.) affinity chromatography using the following protocol for antibody purification. The purified antibody preparation was sterilized by filtration and stored at 4° C.

The antibody purification protocol is as follows: NSO transfectoma cell line producing antibody is grown in DMEM 5% FCS in Nunc Triple layer flasks, 250 ml per flask (total volume 1 L) for 10-14 days until nearing saturation. Conditioned medium collected and spun at 3000 rpm for 5 min in bench centrifuge 5 minutes to remove cells. 1/10^{th} volume 1M Tris-HCl pH 8 (Sigma Cat: T3038) is then added to cell supernatant to make this 0.1 M Tris-HCl pH8. 0.5 to 1 ml Prosep A (Millipore Cat: 113 111824) is added and stirred overnight at room temperature. Prosep A collected by spinning at 3000 rpm for 5 minutes then packed into a Biorad Poly-Prep column (Cat: 73 1-1550). The column is washed with 10 ml PBS, then eluted in 1 ml fractions with 0.1M Glycine pH 3.0. Each fraction is collected into a tube containing 100 microL 1M Tris-HCl pH 8 (Sigma, as above). Absorbance of each fraction is measured at 280 nm. Fractions containing the antibody are pooled and dialyzed against PBS overnight at room temperature. The preparation is sterilized by filtration through a 0.2 micron syringe filter and the absorbance of each fraction is measured at 280 nm. The antibody concentration is determined by ELISA for human IgG.

The purified antibody can be quantified using the protocol for Human IgG1/κ ELISA described above.

Testing of J415 Deimmunized Antibodies

The J415 deimmunized antibodies were tested in an ELISA for binding to LNCap membrane preparation following the protocol as detailed above. ELISA plates were coated with LNCap membrane preparation and blocked with 5% BSA in phosphate buffered saline. Doubling dilutions of the J415 chimeric antibody (murine variable heavy and light chain regions fused to human constant heavy and light chain regions, respectively) and the deimmunized antibodies were applied. Detection was with horseradish peroxidase conjugated goat anti-human IgG and donkey anti-mouse for chimeric and mouse antibodies respectively. Color was developed with o-phenylene diamine substrate.

The antibody composed of deimmunized J415 heavy chain version 4 (also referred to as “J415DIVH4”) combined with deimmunized J415 light chain version 5 (also referred to as “J415DIVK5”) shows equivalent binding to LNCap cells as compared to the chimeric antibody. Also, when DIVK5 is combined with heavy chain versions 1 and 2 (also referred to as “J415DIVH1” and “J415DIVH2”, respectively), binding to LNCap cells is equivalent to that of the chimeric antibody when tissue culture supernatant is analyzed. These data can be confirmed with purified antibody. When light chains 1, 2, 3 were combined with any of the J415 heavy chain versions 1, 2, 3, and 4 no antibody was produced. Deimmunized J415 light chain versions 1, 2, and 3 (“J415DIVK1”, “J415DIVK2”, and “J415DIVK3”, respectively) may be defective on structural grounds. The best chain combination for higher affinity and decreased immunogenicity is DIVH4/DIVK5.

The antibody composed of deimmunized heavy chain version 4 combined with deimmunized light chain version 5 showed equivalent binding to LNCap compared to the chimeric antibody. Also, when DIVK5 is combined with heavy chain versions 1 and 2, binding to LNCap cells is two-fold less than that of the chimeric when purified antibody is analyzed.

In some embodiments, the anti-PSMA antibody, e.g., the modified anti-PSMA antibody or antigen-binding fragment thereof, includes at least one light or heavy chain immunoglobulin (or preferably, at least one light chain immunoglobulin and at least one heavy chain immunoglobulin). Preferably, each immunoglobulin includes a light or a heavy chain variable region having at least one, two and, preferably, three CDRs substantially identical to a CDR from a non-human anti-PSMA light or heavy chain variable region, respectively. For example, the antibody or antigen-binding fragment thereof can have at least one, two and preferably three CDRs from: the heavy chain variable region of murine J591 (see SEQ ID NO:1, 2, and 3, depicted in FIG. 1A); the light chain variable region of murine J591 (see SEQ ID NO:4, 5, and 6, depicted in FIG. 1B); the heavy chain variable region of murine J415 (see SEQ ID NO:29, 30, and 31, depicted in FIG. 5); the light chain variable region of murine J415 (see SEQ ID NO:32, 33, and 34, depicted in FIG. 6); the heavy chain variable region of murine J533 (see SEQ ID NO:93, 94, and 95, depicted in FIG. 9A); the light chain variable region of murine J533 (see SEQ ID NO:96, 97, and 98, depicted in FIG. 10A); the heavy chain variable region of murine E99 (see SEQ ID NO:99, 100, and 101, depicted in FIG. 11A); or the light chain variable region of murine E99 (see SEQ ID NO:102, 103, and 104, depicted in FIG. 12A). In other embodiments, the modified antibody or antigen-binding fragment thereof can have at least one, two, and preferably three CDRs from the light or heavy chain variable region of the antibody produced by the cell line having ATCC Accession Number HB-12126 or the deimmunized J591 antibody produced by the cell line having ATCC Accession Number PTA-3709. In other embodiments, the modified antibody or antigen-binding fragment thereof can have at least one, two and preferably three CDRs from the light or heavy chain variable region of the antibody produced by the cell line having ATCC Accession Number HB-12109 or the deimmunized J415 antibody produced by a cell line having ATCC Accession Number PTA-4174. In still other embodiments, the modified antibody or antigen-binding fragment thereof can have at least one, two and preferably three CDRs from the light or heavy chain variable region of the antibody produced by the cell line having ATCC Accession Number HB-12127 or the antibody produced by a cell line having ATCC Accession Number HB-12101.

In one preferred embodiment, the modified antibody or antigen-binding fragment thereof includes all six CDRs from the same non-human anti-PSMA antibody, e.g., the murine J591, J415, J533 or E99 antibody. In some embodiments, the CDRs have the amino acid sequences of SEQ ID NO:1, 2, 3, 4, 5 and 6 (corresponding to murine J591 heavy and light chain CDRs), the amino acid sequences of the CDRs of the antibody produced by the cell line having ATCC Accession number HB-12126 or the deimmunized J591 antibody produced by the cell line having ATCC Accession Number PTA-3709, or sequences substantially identical thereto. In other embodiments, the CDRs have the amino acid sequences of SEQ ID NO:29, 30, 31, 32, 33, and 34 (corresponding to murine J415 heavy and light chain CDRs), the amino acid sequences of the CDRs of the antibody produced by the cell line having ATCC Accession Number HB-12109 or the deimmunized J415 antibody produced by the cell line having ATCC Accession Number PTA-4174, or sequences substantially identical thereto. In other embodiments, the CDRs have the amino acid sequences of SEQ ID NO:93, 94, 95, 96, 97, and 98 (corresponding to murine J533 heavy and light chain CDRs), the amino acid sequences of the CDRs of the antibody produced by the cell line having ATCC Accession Number HB-12127, or sequences substantially identical thereto. In still other embodiments, the CDRs have the amino acid sequences of SEQ ID NO:99, 100, 101, 102, 103, and 104 (corresponding to murine E99 heavy and light chain CDRs), the amino acid sequences of the CDRs of the antibody produced by the cell line having ATCC Accession Number HB-12101, or sequences substantially identical thereto.

The amino acid sequence of the CDRs for antibodies J591, J415, J533 and E99 are provided below in Table 3.

TABLE 3
CDR Sequences

| | | | | |
|---|---|---|---|---|
| | | | SEQ | |
| | | | ID | |
| NAME | Organism | FIG. | NO: | SEQUENCE |

| | | | | |
|---|---|---|---|---|
| V_{H} CDR1 | Mus musculus | FIG. | 1 | GYTFTEYTIIH |
| J591 | | 1A | | |
| V_{H} CDR2 | Mus musculus | FIG. | 2 | NINPNNGGTTYNQKFED |
| J591 | | 1A | | |
| V_{H} CDR3 | Mus musculus | FIG. | 3 | GWNIFDY |
| J591 | | 1A | | |
| V_{L} CDR1 | Mus musculus | FIG. | 4 | KASQDVGTAVD |
| J591 | | 1B | | |
| V_{L} CDR2 | Mus musculus | FIG. | 5 | WASTRHT |
| J591 | | 1B | | |
| V_{L} CDR3 | Mus musculus | FIG. | 6 | QQYNSYPLT |
| J591 | | 1B | | |
| V_{H} CDR1 | Mus musculus | FIG. | 29 | GFTFSNYWMN |
| J415 | | 5 | | |
| V_{H} CDR2 | Mus musculus | FIG. | 30 | EIIRSQSNNFATHYAESVKG |
| J415 | | 5 | | |
| V_{H} CDR3 | Mus musculus | FIG. | 31 | RWNNF |
| J415 | | 5 | | |
| V_{L} CDR1 | Mus musculus | FIG. | 32 | KASENVGTYVS |
| J415 | | 6 | | |
| V_{L} CDR2 | Mus musculus | FIG. | 33 | GASNRFT |
| J415 | | 6 | | |
| V_{L} CDR3 | Mus musculus | FIG. | 34 | GQSYTFPYT |
| J415 | | 6 | | |
| V_{H} CDR1 | Mus musculus | FIG. | 93 | GYTFTGYVMH |
| J533 | | 9A | | |
| V_{H} CDR2 | Mus musculus | FIG. | 94 | YTNPYNDVTRYNGKFKG |
| J533 | | 9A | | |
| V_{H} CDR3 | Mus musculus | FIG. | 95 | GENWYYFDS |
| J533 | | 9A | | |
| V_{L} CDR1 | Mus musculus | FIG. | 96 | RASESIIIDSYDNTFMH |
| J533 | | 10A | | |
| V_{L} CDR2 | Mus musculus | FIG. | 97 | RASILES |
| J533 | | 10A | | |
| V_{L} CDR3 | Mus musculus | FIG. | 98 | HQSIEDPYT |
| J533 | | 10A | | |
| V_{H} CDR1 | Mus musculus | FIG. | 99 | GFSLTAYGIN |
| E99 | | 11A | | |
| V_{H} CDR2 | Mus musculus | FIG. | 100 | VIWPDGNTDYNSTLKS |
| E99 | | 11A | | |
| V_{H} CDR3 | Mus musculus | FIG. | 101 | DSYGNFKRGWFDF |
| E99 | | 11A | | |
| V_{L} CDR1 | Mus musculus | FIG. | 102 | KASQNVGSDVA |
| E99 | | 12A | | |
| V_{L} CDR2 | Mus musculus | FIG. | 103 | STSYRYS |
| E99 | | 12A | | |
| V_{L} CDR3 | Mus musculus | FIG. | 104 | QQYNSYPLT |
| E99 | | 12A | | |

The light or heavy chain immunoglobulin of the modified anti-PSMA antibody or antigen-binding fragment thereof can further include a light chain or a heavy chain variable framework sequence from a light chain or heavy chain variable framework present in a human or a non-human, e.g., rodent, antibody (e.g., the murine J591, J415, J533 or E99 antibody heavy chain or light chain variable framework). In some embodiments, the light chain or the heavy chain variable framework can be chosen from:

i a light or heavy chain variable framework including at least 5, 10, 20, 30, 40, 50, 60, 70, or 80 amino acid residues from a human light or heavy chain variable framework, e.g., a light or heavy chain variable framework residue from a mature human antibody, a human germline antibody sequence, or a human consensus antibody sequence;

ii a light or heavy chain variable framework including at least 5, but less than 30, amino acid residues from a human light chain or heavy chain variable framework, e.g., a light chain or heavy chain variable framework residue from a mature human antibody, a human germline antibody sequence, or a human consensus antibody sequence;

iii a light or heavy chain variable framework including at least 5, 10, 20, 30, 40, 50, 60, 75 or more amino acid residues from a light or heavy variable framework from a non-human antibody, e.g., a murine antibody (e.g., an anti-PSMA antibody having the framework amino acid sequence shown in SEQ ID NO:7 or 8 (from the heavy and light chain, respectively, of murine J591; see FIGS. 1A and 1B), SEQ ID NO:35 or 36 (from the heavy and light chain, respectively, of murine J415; see FIGS. 5 and 6), SEQ ID NO:109 or 114 (from the heavy and light chain, respectively, of murine J533; see FIGS. 9A and 10A), or SEQ ID NO:119 or 124 (from the heavy and light chain, respectively, of murine E99; see FIGS. 11A and 12A)), or the framework of a murine antibody described herein (e.g., a murine J591, J415, J533, or E99 antibody produced by a hybridoma cell line having an ATCC Accession Number HB-12126, HB-12109, HB-12127 or HB-12101);

iv a light or heavy chain variable framework, which has at least 60%, 65%, 70%, 72%, 75%, 80%, 85%, 90%, 95%, 96%, 97%, 98%, 99% or more identity with, or which has an amino acid sequence which differs by at least 1, 2, 5, or more residues, but less than 10, 20, 30 or 40 residues from, the sequence of the framework of a light or heavy chain variable region of a non-human antibody, e.g., a murine antibody (e.g., an anti-PSMA antibody having the framework amino acid sequence shown in SEQ ID NO:7 or 8 (from the heavy and light chain, respectively, of murine J591; see FIGS. 1A and 1B), SEQ ID NO:35 or 36 (from the heavy and light chain, respectively, of murine J415; see FIGS. 5 and 6), SEQ ID NO:109 or 114 (from the heavy and light chain, respectively, of murine J533; see FIGS. 9A and 10A), or SEQ ID NO:119 or 124 (from the heavy and light chain, respectively, of murine E99; see FIGS. 11A and 12A)), or the framework of a murine antibody described herein (e.g., a murine antibody produced by a hybridoma cell line having an ATCC Accession Number HB-12126, HB-12109, HB-12127 or HB-12101); or

v a non-human, e.g., a murine, e.g., a J591 or J415, light or heavy chain variable region framework which has at least 5 amino acid replacements.

In some embodiments, the light chain variable region of the non-human anti-PSMA antibody or antigen-binding fragment thereof has at least one, two, three and preferably four amino acid sequences chosen from SEQ ID NO:13, 14, 15, and 16 (corresponding to deimmunized J591 light chain FR's 1-4; see FIG. 2B) or SEQ ID NO:41, 42, 43, and 44 (corresponding to deimmunized J415 light chain (J415DIVK5) FR's 1-4; see FIG. 6), or at least one, two, three and preferably four light chain framework regions from the antibody produced by the cell line having ATCC Accession Number PTA-3709 or PTA-4174. In other embodiments, the heavy chain variable region of the non-human anti-PSMA antibody or antigen binding portion thereof has at least one, two, three, and preferably four amino acid sequences chosen from SEQ ID NO:9, 10, 11, and 12 (corresponding to deimmunized J591 heavy chain FR's 1-4; see FIG. 2A) or SEQ ID NO:37, 38, 39, and 40 (corresponding to deimmunized J415 heavy chain (J415DIVH4) FR's 1-4; see FIG. 5), or at least one, two, three and preferably four heavy chain framework regions of the antibody produced by the cell line having ATCC Accession Number PTA-3709 or PTA-4174. In other embodiments, the heavy or light chain framework has an amino acid sequence which has at least 80%, 85%, 90%, 95%, 97%, 98%, 99% or more identity with SEQ ID NO:17 or SEQ ID NO:18, respectively (corresponding to deimmunized J591 framework sequence; see FIGS. 2A-2B), SEQ ID NO:45 or SEQ ID NO:46, respectively (corresponding to deimmunized J415 framework sequences J415DIVH4 and J415DIVK5; see FIG. 5 or 6), or with the heavy or light chain framework sequence of an antibody produced by the cell line having ATCC Accession Number PTA-3709 or PTA-4174. In still other embodiments, the heavy or light chain framework has an amino acid sequence which differs by at least 1, 2, 5, or more residues, but less than 10, 20, 30, or 40 residues, from the amino acid sequence of SEQ ID NO:17 or SEQ ID NO:18, respectively, SEQ ID NO:45 or SEQ ID NO:46, respectively, or the heavy or light chain framework sequence of the antibody produced by the cell line having ATCC Accession Number PTA-3709 or PTA-4174. Preferably, the heavy or light chain framework region includes the amino acid sequence shown in SEQ ID NO:17 or SEQ ID NO:18, respectively, SEQ ID NO:45 or SEQ ID NO:46, respectively, or the heavy or light chain framework sequence of the antibody produced by the cell line having ATCC Accession Number PTA-3709 or PTA-4174.

In other embodiments, the heavy or light chain variable region of the modified anti-PSMA antibody has an amino acid sequence which has at least 80%, 85%, 90%, 95%, 97%, 98%, 99% or more identity with SEQ ID NO:21 or SEQ ID NO:22, respectively (corresponding to the heavy and light chain variable regions of deimmunized J591; see FIGS. 2A-2B), SEQ ID NO:49 or SEQ ID NO:50, respectively (corresponding to the heavy and light chain variable regions of deimmunized J415, J415DIVH4 and J415DIVK5; see FIG. 5 or 6), or the heavy or light chain variable region sequence of the antibody produced by the cell line having ATCC Accession Number PTA-3709 or PTA-4174. In other embodiments, the heavy or light chain variable region of the modified anti-PSMA antibody has an amino acid sequence that differs by at least 1, 2, 5, or more residues, but less than 10, 20, 30, or 40 residues, from the amino acid sequence of SEQ ID NO:21 or SEQ ID NO:22, respectively, SEQ ID NO:49 or SEQ ID NO:50, respectively, or the heavy or light chain variable region sequence of the antibody produced by the cell line having ATCC Accession Number PTA-3709 or PTA-4174. Preferably, the light or heavy chain variable region includes the amino acid sequence shown in SEQ ID NO:21 or SEQ ID NO:22, respectively, SEQ ID NO:49 or SEQ ID NO:50, respectively, or the heavy or light chain variable region sequence of the antibody produced by the cell line having ATCC Accession Number PTA-3709 or PTA-4174.

Preferred modified anti-PSMA antibodies include at least one, preferably two, light chain variable regions and at least one, preferably two, heavy chain variable regions having the amino acid sequence shown in SEQ ID NO:21 and SEQ ID NO:22, respectively (corresponding to the heavy and light chain variable regions of deimmunized J591; see FIGS. 2A-2B), SEQ ID NO:49 and SEQ ID NO:50, respectively (corresponding to the heavy and light chain variable regions of deimmunized J415, J415DIVH4 and J415DIVK5; see FIGS. 5 and 6), or at least one, preferably two, modified light chain variable region sequences and at least one, preferably two, heavy chain variable region sequences of the antibody produced by the cell line having ATCC Accession Number PTA-3709 or PTA-4174.

In other embodiments, the light or heavy chain variable framework of the anti-PSMA antibody, or antigen-binding fragment thereof, includes at least one, two, three, four, five, six, seven, eight, nine, ten, fifteen, sixteen, or seventeen amino acid residues from a human light or heavy chain variable framework, e.g., a light or heavy chain variable framework residue from a mature human antibody, a human germline antibody sequence, or a consensus antibody sequence.

In some embodiments, the amino acid residue from the human light chain variable framework is the same as the residue found at the same position in a human germline antibody sequence. Preferably, the amino acid residue from the human light chain variable framework is the most common residue at the same position in the human germline antibody sequence. Preferably, the light chain variable framework of the modified anti-PSMA antibody, or antigen-binding fragment thereof, has at least one, two, three, five, seven, ten amino acid residues which differ from the framework of the non-human anti-PSMA light chain variable region (e.g., the murine J591 light chain variable region), or which is from a human light chain variable framework (e.g., a human germline, mature, or consensus framework sequence), at a position selected from the group consisting of: residue 8, 9, 10, 11, 20, 22, 60, 63, 76, 77, 78, 80, 83, 87, 103, 104 and 106 (Kabat numbering as shown in Table 4). Preferably, the light chain variable framework of the modified anti-PSMA antibody, or antigen-binding fragment thereof, has at least one, two, three, five, seven, or ten amino acid residues from the human light chain variable framework selected from the group consisting of: residue 8 (proline), 9 (serine), 10 (serine), 11 (leucine), 20 (threonine), 22 (threonine), 60 (serine), 63 (serine), 76 (serine), 77 (serine), 78 (leucine), 80 (proline), 83 (phenylalanine), 87 (tyrosine), 103 (lysine), 104 (valine) and 106 (isoleucine) (Kabat numbering as shown in Table 4).

The amino acid replacements in the deimmunized J591 light chain variable region are provided below in Table 4. The left panel indicates the amino acid number according to Kabat, E. A., et al. (1991) supra; the middle panel indicates the replacements of the residue in the mouse sequence and the corresponding mouse residues; and the right panel indicates the most common residue in the corresponding position in the human germline.

TABLE 4

| Position | Substitution | Most common in |
|---|---|---|
| Kabat No. | of mouse sequence | human germline |
| 3 | V→Q | V |
| 8 | H→P | P |
| 9 | K→S | S |
| 10 | F→S | S |
| 11 | M→L | L |
| 20 | S→T | T |
| 21 | I→L | I |
| 22 | I→T | T |
| 42 | Q→P | K |
| 58 | V→I | V |
| 60 | D→S | S |
| 63 | T→S | S |
| 76 | T→S | S |
| 77 | T→S | S |
| 78 | V→L | L |
| 80 | S→P | P |
| 83 | L→F | F |
| 87 | F→Y | Y |
| 100 | A→P | Q |
| 103 | M→K | K |
| 104 | L→V | V |
| 106 | L→I | I |

In other embodiments, the light chain variable framework of the anti-PSMA antibody, or antigen-binding fragment thereof, has at least one, two, three, five, or seven amino acid residues which differ from the framework of a non-human anti-PSMA light chain variable region (e.g., the murine J415 light chain variable region), or which is from a human light chain variable framework (e.g., a human germline, mature, or consensus framework), at a position selected from the group consisting of: residue 13, 15, 19, 41, 63, 68, and 80 (linear numbering as shown in FIG. 6 and Table 5). Preferably, the light chain variable framework of the modified antibody, or antigen-binding fragment thereof, has at least one, two, three, five, or seven amino acid residues from the human consensus light chain variable framework selected from the group consisting of: residue 13 (alanine), 15 (alanine), 19 (methionine), 41 (threonine), 63 (serine), 68 (glycine), and 80 (alanine) (linear numbering as shown in FIG. 6 and Table 5).

The amino acid replacements in one of the deimmunized J415 light chain variable region are provided below in Table 5. The left panel indicates the amino acid number using linear numbering; the middle panel indicates the replacements of the residue in the mouse sequence and the corresponding mouse residues; and the right panel indicates the most common residue in the corresponding position in the human germline.

TABLE 5

| Position | Substitution | Most common in |
|---|---|---|
| Linear No | of mouse sequence | human germline |
| 13 | I→A | A |
| 15 | V→A | A |
| 19 | V→M | M |
| 41 | E→T | T |
| 63 | T→S | S |
| 68 | A→G | G |
| 80 | T→A | A |

In other embodiments, the light chain variable framework of the anti-PSMA antibody, or antigen-binding fragment thereof, includes at least 5, but no more than 80, amino acid residues from the light chain variable framework shown in SEQ ID NO:8 (from murine J591; see FIG. 1B), SEQ ID NO:36 (from murine J415; see FIG. 6), SEQ ID NO:114 (from murine J533; see FIG. 10A), or SEQ ID NO:124 (from murine E99; see FIG. 12A), or the light chain variable framework of an antibody produced by the hybridoma cell line having an ATCC Accession Number HB-12126, HB-12109, HB-12127 or HB-12101. Preferably, the light chain variable framework has at least 60%, 65%, 70%, 72%, 75%, 80%, 85%, 90%, or 94% identity with, or differs by at least 5, 7, 10, 20, or 30 but less than 10, 20, 30, or 40 amino acid residues from, the non-human light chain variable framework, e.g., the murine J591 or J415 light chain variable framework shown in SEQ ID NO:8 or SEQ ID NO:36, respectively, or the light chain variable framework of the antibody produced by the hybridoma cell line having an ATCC Accession Number HB-12126 or HB-12109. In other embodiments, the light chain variable framework is from murine J591 antibody (SEQ ID NO:8; see FIG. 1B), from murine J415 antibody (SEQ ID NO:36; see FIG. 6), from murine J533 antibody (SEQ ID NO:114; see FIG. 10A), or from murine E99 antibody (SEQ ID NO:124; see FIG. 12A), or the light chain variable framework of the antibody produced by the hybridoma cell line having an ATCC Accession Number HB-12126, HB-12109, HB-12127 or HB-12101.

In yet other embodiments, the light chain variable framework of the modified anti-PSMA antibody, or antigen-binding fragment thereof, includes a non-human (e.g., a murine) light chain variable framework (e.g., a murine J591 light chain variable framework as shown in SEQ ID NO:8 or the light chain variable framework of the antibody produced by the hybridoma cell line having an ATCC Accession Number HB-12126) which has at least 5, 10, 15, 16, 17, 18, 19, 20, 21, 22, or 23 amino acid replacements. In one embodiment, the non-human light chain variable framework includes one or more of:

a framework region 1 having at least 5, 6, 7, or 8 replacements;

a framework region 2 having at least one replacement;

a framework region 3 having at least 5, 6, 7, 8, or 9 replacements; or

a framework region 4 having at least 2, 3 or 4 replacements.

In yet other embodiments, the light chain variable framework of the modified anti-PSMA antibody, or antigen-binding fragment thereof, includes a non-human (e.g., a murine) light chain variable framework (e.g., a murine J415 light chain variable framework as shown in SEQ ID NO:36 or the light chain variable framework of the antibody produced by the hybridoma cell line having an ATCC Accession Number HB-12109) which has at least 1, 2, 3, 4, 5, 6, 7, 8, or 10 amino acid replacements. In some embodiments, the non-human light chain variable framework includes one or more of:

a framework region 1 having at least 1, 2 or 3 replacements;

a framework region 2 having at least one replacement; or

a framework region 3 having at least 1, 2 or 3 replacements.

The replacement can be selected from: a conservative substitution of a non-human residue, or a residue found in a human germline, mature or consensus framework sequence at the same position, e.g. the most common residue in the human germline sequence at the same position. In some embodiments, the light chain variable framework has at least 3, 4 and preferably 5 conservative substitutions. In other embodiments, the light chain variable framework has at least 5, 7, 10, 15, 16, or 17 amino acid replacements wherein the replacement amino acid residue is the most common residue in the human germline framework sequence at the same position.

In some embodiments, the non-human light chain variable framework (e.g., a murine J591 light chain variable framework as shown in SEQ ID NO:8 or the light chain variable framework of the antibody produced by the hybridoma cell line having an ATCC Accession No.: HB-12126) has at least one, two, three, five, seven, ten, eleven, fifteen, sixteen, seventeen, nineteen, twenty, twenty-one or twenty-two amino acid replacements at a position selected from the group consisting of: residue 3, 8, 9, 10, 11, 20, 21, 22, 42, 58, 60, 63, 76, 77, 78, 80, 83, 87, 100, 103, 104 and 106 (Kabat numbering as shown in Table 4). The replacement can be chosen from one or more of: residue 3 (glutamine), 8 (proline), 9 (serine), 10 (serine), 11 (leucine), 20 (threonine), 21 (leucine), 22 (threonine), 42 (proline), 58 (isoleucine), 60 (serine), 63 (serine), 76 (serine), 77 (serine), 78 (leucine), 80 (proline), 83 (phenylalanine), 87 (tyrosine), 100 (proline), 103 (lysine), 104 (valine) and 106 (isoleucine) (Kabat numbering as shown in Table 4).

In other embodiments, the non-human light chain variable framework (e.g., a murine J591 light chain variable framework as shown in SEQ ID NO:36 or the light chain variable framework of the antibody produced by the hybridoma cell line having an ATCC Accession No.: HB-12109) has at least one, two, three, five, or seven amino acid replacements at a position selected from the group consisting of: residue 13, 15, 19, 41, 63, 68 and 80 (linear numbering as shown in FIG. 6 and Table 5). Preferably, the light chain variable framework of the modified antibody, or antigen-binding fragment thereof, has at least one, two, three, five, seven amino acid residues from the human consensus light chain variable framework selected from the group consisting of: residue 13 (alanine), 15 (alanine), 19 (methionine), 41 (threonine), 63 (serine), 68 (glycine) and 80 (alanine) (linear numbering as shown in FIG. 6 and Table 5).

Preferably, the heavy chain variable framework of the modified anti-PSMA antibody, or antigen-binding fragment thereof, has at least one, two, three, five, seven, or eight amino acid residues, which differ from the framework of the non-human anti-PSMA heavy chain variable region (e.g., the murine J591 heavy chain variable region), or which is from a human heavy chain variable framework (e.g., a human germline framework), at a position selected from the group consisting of: residue 5, 40, 41, 44, 82a, 83, 87, and 108 (Kabat numbering as shown in Table 6). Preferably, the heavy chain variable framework of the recombinant antibody, or antigen-binding fragment thereof, has at least one amino acid residue from the human heavy chain variable framework selected from the group consisting of: residue 5 (valine), 40 (alanine), 41 (proline), 44 (glycine), 82a (serine), 83 (arginine), 87 (threonine), or 108 (leucine) (Kabat numbering as shown in Table 6).

The amino acid replacements in the deimmunized J591 heavy chain variable region are provided below in Table 6. The left panel indicates the amino acid number according to Kabat, E. A., et al. (1991) supra; the middle panel indicates the replacements of the residue in the mouse sequence and the corresponding mouse residues; and the right panel indicates the most common residue in the corresponding position in the human germline.

TABLE 6

| Position | Substitution | Most common in |
|---|---|---|
| Kabat No. | of mouse sequence | human germline |
| 5 | Q→V | V |
| 11 | L→V | L |
| 12 | V→K | V |
| 16 | T→A | G |
| 17 | S→T | S |
| 19 | R→K | R |
| 40 | S→A | A |
| 41 | H→P | P |
| 44 | S→G | G |
| 75 | S→T | K |
| 76 | S→D | N |
| 82a | R→S | S |
| 83 | T→R | R |
| 87 | S→T | T |
| 108 | T→L | L |

In other embodiments, the heavy chain variable framework of the modified anti-PSMA antibody, or antigen-binding fragment thereof, has at least one, two, three, four, five amino acid residues, which differ from the framework of a non-human anti-PSMA heavy chain variable region (e.g., the murine J415 heavy chain variable region), or which is from a human heavy chain variable framework (e.g., a human mature, consensus, or germline framework), at a position selected from the group consisting of: residue 20, 87, 94, 95, and 112 (linear numbering as shown in FIG. 5 and in Table 7). Preferably, the heavy chain variable framework of the recombinant antibody, or antigen-binding fragment thereof, has at least one, two, three, four, five amino acid residue from the human heavy chain variable framework selected from the group consisting of: residue 20 (isoleucine), 87 (serine), 94 (alanine), 95 (valine), and 112 (valine) (linear numbering as shown in FIG. 5 and in Table 7).

The amino acid replacements in one of the deimmunized J415 heavy chain variable region are provided below in Table 7. The left panel indicates the linear amino acid number; the middle panel indicates the replacements of the residue in the mouse sequence and the corresponding mouse residues; and the right panel indicates the most common residue in the corresponding position in the human germline.

TABLE 7

| Position | Substitution | Most common in |
|---|---|---|
| Kabat No | of mouse sequence | human germline |
| 20 | L→I | I |
| 87 | N→S | S |
| 94 | G→A | A |
| 95 | I→V | V |
| 112 | L→V | V |

In other embodiments, the heavy chain variable framework of the modified anti-PSMA antibody, or antigen-binding fragment thereof, includes at least 5 but no more than 75 or 82 amino acid residues from the heavy chain variable framework shown in SEQ ID NO:7 (from murine J591; see FIG. 1A), SEQ ID NO:35 (from murine J415; see FIG. 5), SEQ ID NO:109 (from murine J533; see FIG. 9A), or SEQ ID NO:119 (from murine E99; see FIG. 11A), or the heavy chain variable framework of the antibody produced by the hybridoma cell line having an ATCC Accession Number HB-12126, HB-12109, HB-12127 or HB-12101. Preferably, the heavy chain variable framework has at least 60%, 65%, 70%, 80%, 82%, 85%, 90%, or 94% identity with, or differs by at least 5, 10, 20, or 30 but less than 10, 20, 30, or 40 residues from, a non-human heavy chain variable framework, e.g., the murine J591 or J415 or heavy chain variable framework shown in SEQ ID NO:7 or SEQ ID NO:35, respectively, or a heavy chain variable framework of the antibody produced by the hybridoma cell line having an ATCC Accession Number HB-12126 or HB-12109, respectively. In other embodiments, the non-human heavy chain variable framework is from murine J591 antibody (SEQ ID NO:7; see FIG. 1A), from murine J415 antibody (SEQ ID NO:35; see FIG. 5), from murine J533 antibody (SEQ ID NO:109; see FIG. 9A), or from murine E99 antibody (SEQ ID NO:119; see FIG. 11A), or the heavy chain variable framework of the antibody produced by the hybridoma cell line having an ATCC Accession Number HB-12126, HB-12109, HB-12127 or HB-12101.

In yet other embodiments, the heavy chain variable framework of the modified anti-PSMA antibody, or antigen-binding fragment thereof, includes a non-human (e.g., a murine) heavy chain variable framework (e.g., a murine J591 heavy chain variable framework (SEQ ID NO:7, as shown FIG. 1A, or the heavy chain variable framework of the antibody produced by the hybridoma cell line having an ATCC Accession Number HB-12126) which has at least 3, 5, 10, 15, 16, 17, 18, or 19 amino acid replacements. In one embodiment, the non-human heavy chain variable framework of the modified anti-PSMA antibody includes one or more of:

a framework region 1 having at least 4, 5, or 6 replacements;

a framework region 2 having at least 1, 2, or 3 replacements;

a framework region 3 having at least 3, 4, or 5 replacements; or

a framework region 4 having at least one replacement.

In yet other embodiments, the heavy chain variable framework of the modified anti-PSMA antibody, or antigen-binding fragment thereof, includes a non-human (e.g., a murine) heavy chain variable framework (e.g., a murine J415 heavy chain variable framework (SEQ ID NO:35, as shown in FIG. 5, or the heavy chain variable framework of the antibody produced by the hybridoma cell line having an ATCC Accession Number HB-12109) which has at least 1, 2, 3, 4, or 5 amino acid replacements. In one embodiment, the non-human heavy chain variable framework of the modified anti-PSMA antibody includes one or more of:

a framework region 1 having at least one replacement;

a framework region 3 having at least 1, 2, or 3 replacements; or

a framework region 4 having at least one replacement.

The replacement can be chosen from: a conservative substitution of a non-human residue, or a residue found in a human germline, mature or consensus sequence at the same position, e.g. the most common residue in the human germline at the same position. In one embodiment, the heavy chain variable framework has at least 3, 4, 5, 6 and preferably 7 conservative substitutions. Preferably, the heavy chain variable framework has at least 5, 6, 7 and preferably 8 replacements by the most common residue in the human germline at the same position.

In some embodiments, the non-human heavy chain variable framework (e.g., a murine J591 heavy chain variable framework of SEQ ID NO:7 or the heavy chain variable framework of the antibody produced by the hybridoma cell line having an ATCC Accession Number HB-12126) has at least one amino acid replacement at a position selected from the group consisting of: residue 5, 11, 12, 16, 17, 19, 40, 41, 44, 75, 76, 82a, 83, 87, and 108 (Kabat numbering as shown in Table 6). The replacement can be chosen from one or more of: 5 (valine), 11 (valine), 12 (lysine), 16 (alanine), 17 (threonine), 19 (lysine), 40 (alanine), 41 (proline), 44 (glycine), 75 (threonine), 76 (aspartate), 82a (serine), 83 (arginine), 87 (threonine), and 108 (leucine) (Kabat numbering as shown in Table 6).

In other embodiments, the non-human heavy chain variable framework (e.g., a murine J415 heavy chain variable framework of SEQ ID NO:35 or the heavy chain variable framework of the antibody produced by the hybridoma cell line having an ATCC Accession Number HB-12109) has at least one amino acid replacement at a position selected from the group consisting of: residue 20, 87, 94, 95 and 112 (linear numbering as shown in FIG. 5 and in Table 7). The replacement can be chosen from one or more of: residue 20 (isoleucine), 87 (serine), 94 (alanine), 95 (valine), and 112 (valine) (linear numbering as shown in FIG. 5 and in Table 7).

The amino acid sequence of the framework regions of the light and heavy chains regions of antibodies J591, J415, J533 and E99 are provided in Table 8, below.

TABLE 8
Framework Sequences

| | | | | |
|---|---|---|---|---|
| | | | SEQ ID: | |
| NAME | Organism | FIG. | NO: | SEQUENCE |

| | | | | |
|---|---|---|---|---|
| V_{H} FR1-FR4 | Mus musculus | | 7 | EVQLQQSGPELKKPGTSVRISCK |
| J591 | | | | TSWVKQSHGKSLEWIGKATLTV |
| | | | | DKSSSTAYMELRSLTSEDSAVY |
| | | | | YCAAWGQGTTLTVSS |
| V_{L} FR1-FR4 | Mus musculus | | 8 | DIVMTQSHKFMSTSVGDRVSIIC |
| J591 | | | | WYQQKPGQSPKLLIYGVPDRFT |
| | | | | GSGSGTDFTLTITNVQSEDLADY |
| | | | | FCFGAGTMLDLK |
| V_{H} FRi (Deimm) | Artificial-deimmunized | | 9 | EVQLVQSGPEVKKPGATVKISC |
| J591 | | | | KTS |
| | heavy chain | | | |
| | J591 | | | |
| V_{H} FR2 (Deimm) | Artificial-deimmunized | | 10 | WVKQA |
| PGKGLEWIG | | | | |
| J591 | | | | |
| heavy chain | | | | |
| J591 | | | | |
| V_{H} FR3 (Deimm) | Artificial-deimmunized | | 11 | KATLTVDKSTDTAYMELSSLRS |
| J591 | | | | EDTAVYYGAA |
| | heavy chain | | | |
| | J591 | | | |
| V_{H} FR4 (Deimm) | Artificial-deimmunized | | 12 | WGQGTLLTVSS |
| J591 | | | | |
| | heavy chain | | | |
| | J591 | | | |
| V_{L} FR1 (Deimm) | Artificial-deimmunized | | 13 | DIQMTQSPSSLSTSVGDRVTLTC |
| J591 | | | | |
| | light chain J591 | | | |
| V_{L} FR2 (Deimm) | Artificial-deimmunized | | 14 | WYQQKPGPSPKLLIY |
| J591 | | | | |
| | light chain J591 | | | |
| V_{L} FR3 (Deimm) | Artificial-deimmunized | | 15 | GTPSRFSGSGSGTDFTLTISSLQPE |
| J591 | | | | DFADYYC |
| | light chain J591 | | | |
| V_{L} FR4 (Deimm) | Artificial-deimmunized | | 16 | FGPGTKVDIK |
| 3591 | | | | |
| | light chain J591 | | | |
| V_{H} FR1-FR4 | Artificial-deimmunized | | 17 | EVQLVQSGPEVKKPGATVKISG |
| (Deimm) | | | | KTSWVKQAPGKGLEWIGKATLT |
| J591 | heavy chain | | | VDKSTDTAYMELSSLRSEDTAV |
| | J591 | | | YYCAAWGQGTLLTVSS |
| V_{L} FR1-FR4 | Artificial-deimmunized | | 18 | DIQMTQSPSSLSTSVGDRVTLTC |
| (Deimm) | | | | WYQQKPGPSPKLLIYGLPSRFSGS |
| J591 | light chain J591 | | | GSGTDFTLTISSLQPEDFADYYC |
| | | | | FGPGTKVDIK |
| V_{H} FRI-FR4 | Mus musculus | | 35 | EVKLEESGGGLVQPGGSMKLSC |
| J415 | | | | VASWVRQSPEKGLEWVARVIIS |
| | | | | RDDSKSSVYLQMNNLRAEDTGI |
| | | | | YYCTRWG GTFITLTVSS |
| V_{L} FR1-FR4 | Mus musculus | | 36 | N1VMTQFPKSMSISVGERVTLTC |
| J415 | | | | WYQQKPEQSPKMLIYGVPDRFT |
| | | | | GSGSATDFILTISSVQTEDLVDY |
| | | | | YCFGGGTKLEMK |
| V_{H} FR1 (Deimm) | Artificial-deimmunized | | 37 | EVKLEESGGGLVQPGGSMKISC |
| J415-4 | | | | VAS |
| | heavy chain | | | |
| | J415-4 | | | |
| V_{H} FR2 (Deimm) | Artificial-deimmunized | | 38 | WVRQSPEKGLEWVA |
| J415-4 | | | | |
| | heavy chain | | | |
| | J415-4 | | | |
| V_{H} FR3 (Deimm) | Artificial-deimmunized | | 39 | RVHSRDDSKSSVYLQMNSLRAE |
| J415-4 | | | | DTAVYYCTR |
| | heavy chain | | | |
| | J415-4 | | | |
| V_{H} FR4 (Deimm) | Artificial-deimmunized | | 40 | WGQGTTVTVSS |
| J415-4 | | | | |
| | heavy chain | | | |
| | J415-4 | | | |
| V_{L} FRI (Deimm) | Artificial-deimmunized | | 41 | NIVMTQFPKSMSASAGERMTLT |
| J415 | | | | C |
| | light chain | | | |
| | J415-5 | | | |
| V_{L} FR2 (Deimm) | Artificial-deimmunized | | 42 | WYQQKLPTQSPKiML1Y |
| J415-5 | | | | |
| | light chain | | | |
| | J415-5 | | | |
| V_{L} FR3 (Deimm) | Artificial-deimmunized | | 43 | GVPDRFSGSGSGTDFILTISSVQA |
| J415-5 | | | | EDLVDYYG |
| | light chain | | | |
| | J415-5 | | | |
| V_{L} FR4 (Deinim) | Artificial-deimmunized | | 44 | FGGGTKLEMK |
| J415-5 | | | | |
| | light chain | | | |
| | J415-5 | | | |
| V_{H} FR1-FR4 | Artificial-deimmunized | | 45 | EVKLEESGGGLVQPGGSMKISC |
| (Deimm) | | | | VASWVRQSPEKGLEWVARVHS |
| J415-4 | heavy chain | | | RDDSKSSVYLQMNSLRAEDTAV |
| | J415-4 | | | YYCTRWGQGTFITVTVSS |
| V_{L} FR1-FR4 | Artificial-deimmunized | | 46 | NIVMTQFPKSMSASAGERMTLT |
| (Deimm) | | | | CWYQQKPTQSPKMLIYGVPDRIF |
| J415-5 | light chain | | | SGSGSGTDFILTISSVQAEDLVDY |
| | J415-5 | | | YCFGGGTKLEMK |
| V_{H} FRi | Mus musculus | | 105 | EVQLQQSGPELVKPGASVKMSC |
| J533 | | | | KAS |
| V_{H} FR2 | Mus musculus | | 106 | WVKQKPGQ VLE WIG |
| J533 | | | | |
| V_{H} FR3 | Mus musculus | | 107 | KATLTSDKYSSTAYMELSGLTSE |
| J533 | | | | DSAVYYCAR |
| V_{H} FR4 | Mus musculus | | 108 | WGRGATLTVSS |
| J533 | | | | |
| V_{H} FR1-FR4Mus musculus | | 109 | EVQLQQSGPELVKPGASVKMSC | |
| J533 | | | | KASWVKQKPGQVLEWIGKATLT |
| | | | | SDKYSSTAYMELSGLTSEDSAV |
| | | | | YYCARWGRGATLTVSS |
| V_{L} FR1 | Mus musculus | FIG. | 110 | DIVLTQSPASLAVSLGQRATISC |
| J533 | | 10A | | |
| V_{L} FR2 | Mus musculus | FIG. | 111 | WYQQKPGQPPNLLIF |
| J533 | 10A | | | |
| V_{L} FR3 | Mus musculus | FIG. | 112 | GIIPARFSGSGSGTDFTLTIYPVEA |
| J533 | | 10A | | DDVATYYC |
| V_{L} FR4 | Mus musculus | FIG. | 113 | FGGGTKLEIK |
| J533 | | 10A | | |
| V_{L} FR1-FR4Mus musculus | | DIVLTQSPASLAVSLGQRATISC | | |
| J533 | | 10A | | WYQQKPGQPPNLLIFGIPARFSG |
| | | | | SGSGTDFTLTIYPVEADDVATYY |
| | | | | CFGGGTKLER |
| V_{H} FRi | Mus musculus | FIG. | 115 | QVQLKESGPGLVASSQSLSITCT |
| E99 | | 11A | | VS |
| V_{H} FR2 | Mus musculus | FIG. | 116 | WVRQPPGKGLEWLG |
| E99 | | 11A | | |
| V_{H} FR3 | Mus musculus | FIG. | 117 | RLNIFKDNSKNQVFLKMSSFQTD |
| E99 | | 11A | | DTARYFCAR |
| V_{H} FR4 | Mus musculus | FIG. | 118 | WGQGTTLTVSS |
| E99 | | | 11A | |
| V_{H} FR1-FR4 | Mus musculus | FIG. | 119 | QVQLKESGPGLVASSQSLSITCT |
| E99 | | 11A | | VSWVRQPPGKGLEWLGRLNWK |
| | | | | DNSKNQVFLKMSSFQTDDTARY |
| | | | | FCARWGQGTITLTVSS |
| V_{L} FR1 | Mus musculus | FIG. | 120 | NIVMTQSQKFMSTSPGDRVRVT |
| E99 | | 12A | | C |
| V_{L} FR2 | Mus musculus | FIG. | 121 | WYQAKPGQSPRTLIY |
| E99 | | 12A | | |
| V_{L} FR3 | Mus musculus | FIG. | 122 | GVPDRFTAYGSGTDFTLTITNVQ |
| E99 | | 12A | SEDLTEYFC | |
| V_{L} FR4 | Mus musculus | FIG. | 123 | FGAGTKLELK |
| E99 | | 12A | | |
| V_{L} FR1-FR4 | Mus musculus | FIG. | 124 | NIVMTQSQKFMSTSPGDRVRVT |
| E99 | | 12A | | CWYQAKPGQSPRILIYGVPDRFT |
| | | | | AYGSGTDFTLTITNVQSEDLTEY |
| | | | | FCFGAGTKLELK |

In other embodiments, the anti-PSMA antibody, or antigen-binding fragment thereof, includes at least one light chain or heavy chain immunoglobulin or, preferably, at least one light chain immunoglobulin and at least one heavy chain immunoglobulin. Preferably, the light chain immunoglobulin includes a non-human light chain variable region comprising three CDRs from a non-human, e.g., murine, anti-PSMA light chain variable region (e.g., the murine J591 or J415 light chain variable region shown in SEQ ID NO:20 (see FIG. 11B) or SEQ ID NO:48 (see FIG. 6), respectively, or the light chain variable region of the antibody produced by the hybridoma cell line having an ATCC Accession N u m b e r HB-12126 or HB-12109) and a light chain framework which differs from the framework of the non-human, e.g., murine, anti-PSMA light chain framework (e.g., the murine J591 of J415 light chain framework shown in SEQ ID NO:8 (see FIG. 1B) or SEQ ID NO:36 (see FIG. 6), respectively, or the light chain variable framework of the antibody produced by the hybridoma cell line having an ATCC Accession Number HB-12126 or HB-12109) at one, two, three, four, five, six, seven or more positions selected from the group consisting of: residue 3, 8, 9, 10, 11, 20, 21, 22, 42, 58, 60, 63, 76, 77, 78, 80, 83, 87, 100, 103, 104 and 106 (Kabat numbering as in Table 4), or residues 13, 15, 19, 41, 63, 68, and 80 (linear numbering as in FIG. 6 and in Table 5).

In other preferred embodiments, the heavy chain immunoglobulin includes a non-human heavy chain variable region comprising three complementarity determining regions (CDRs) from a non-human, e.g., murine, anti-PSMA heavy chain variable region (e.g., the murine J591 or J415 heavy chain variable region shown in SEQ ID NO:19 (see FIG. 1A) or SEQ ID NO:47 (see FIG. 5), respectively, or the heavy chain variable region of the antibody produced by the hybridoma cell line having an ATCC Accession Number HB-12126 or HB-12109, respectively) and a modified heavy chain framework which differs from the framework of the non-human, e.g., murine, anti-PSMA heavy chain framework (e.g., the murine J591 or J415 heavy chain framework shown in SEQ ID NO:7 (see FIG. 1A) or SEQ ID NO:35 (see FIG. 5), respectively, or the heavy chain variable framework of the antibody produced by the hybridoma cell line having an ATCC Accession Number HB-12126 or HB-12109, respectively) at one, two, three, four, five or more positions selected from the group consisting of: residue 5, 11, 12, 16, 17, 19, 40, 41, 44, 75, 76, 82a, 83, 87, and 108 (Kabat numbering as in Table 5), or residue 20, 87, 94, 95 and 112 (linear numbering as in FIG. 5 and in Table 7).

In yet other embodiments, the modified anti-PSMA antibody, or antigen-binding fragment thereof, includes at least one light or heavy chain immunoglobulin or, more preferably, at least one light chain immunoglobulin and at least one heavy chain immunoglobulin. Preferably, the light chain immunoglobulin includes a modified non-human light chain variable region comprising three CDRs from a non-human, e.g., murine, anti-PSMA light chain variable region (e.g., the murine J591 light chain variable region shown in SEQ ID NO:20 (see FIG. 1B), or the light chain variable region of the antibody produced by the hybridoma cell line having an ATCC Accession Number HB-12126) and a modified light chain framework which differs from the framework of the non-human anti-PSMA light chain variable region, e.g., the murine J591 light chain variable region (SEQ ID NO:20 or the light chain variable region of the antibody produced by the hybridoma cell line having an ATCC Accession Number HB-12126), by at least one, two, three, four, five, six, seven, eight, nine, ten positions selected from the group consisting of:

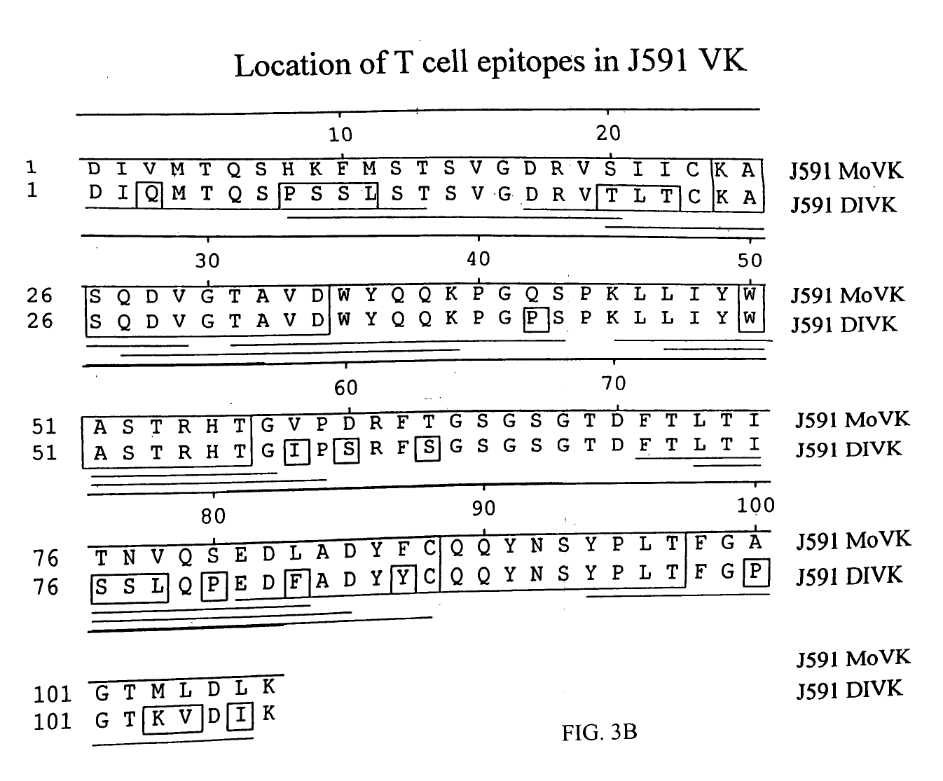

a position within or adjacent to one or more of residues 1, 2, 3, 4, 5, 6, 7, 8, 9, 10, 11, 12 or 13, or a T cell epitope which includes one or more of residues 1-13 (numbering as in FIG. 3B);

a position within or adjacent to one or more of residues 8, 9, 10, 11, 12, 13, 14, 15, 16, 17, 18, 19 or 20, or a T cell epitope which includes one or more of residues 8-20 (numbering as in FIG. 3B);

a position within or adjacent to one or more of residues 17, 18, 19, 20, 21, 22, 23, 24, 25, 26, 27, 28 or 29, or a T cell epitope which includes one or more of residues 17-29 (numbering as in FIG. 3B);

a position within or adjacent to one or more of residues 27, 28, 29, 30, 31, 32, 33, 34, 35, 36, 37, 38 or 39, or a T cell epitope which includes one or more of residues 27-39 (numbering as in FIG. 3B);

a position within or adjacent to one or more of residues 30, 31, 32, 33, 34, 35, 36, 37, 38, 39, 40, 41, 42 or 43, or a T cell epitope which includes one or more of residues 30-43 (numbering as in FIG. 3B);

a position within or adjacent to one or more of residues 45, 46, 47, 48, 49, 50, 51, 52, 53, 54, 55, 56, or 57, or a T cell epitope which includes one or more of residues 45-57 (numbering as in FIG. 3B);

a position within or adjacent to one or more of residues 56, 57, 58, 59, 60, 61, 62, 63, 64, 65, 66, 67, or 68, or a T cell epitope which includes one or more of residues 56-68 (numbering as in FIG. 3B);

a position within or adjacent to one or more of residues 71, 72, 73, 74, 75, 76, 77, 78, 79, 80, 81, 82, or 83, or a T cell epitope which includes one or more of residues 71-83 (numbering as in FIG. 3B);

a position within or adjacent to one or more of residues 73, 74, 75, 76, 77, 78, 79, 80, 81, 82, 83, 84 or 85, or a T cell epitope which includes one or more of residues 73-85 (numbering as in FIG. 3B); and

a position within or adjacent to one or more of residues 94, 95, 96, 97, 98, 99, 100, 101, 102, 103, 104, 105, or 106, or a T cell epitope which includes one or more of residues 94-106 (numbering as in FIG. 3B).

In yet other embodiments, the anti-PSMA antibody, or antigen-binding fragment thereof, includes at least one light or heavy chain immunoglobulin or, more preferably, at least one light chain immunoglobulin and at least one modified heavy chain immunoglobulin. Preferably, the light chain immunoglobulin includes a modified non-human light chain variable region comprising three CDRs from a non-human, e.g., murine, anti-PSMA light chain variable region (e.g., the murine J415 light chain variable region shown in SEQ ID NO:48 (FIG. 6), or the light chain variable region of the antibody produced by the hybridoma cell line having an ATCC Accession Number HB-12109) and a light chain framework which differs from the framework of the non-human anti-PSMA light chain variable region, e.g., the murine J415 light chain variable region (SEQ ID NO:48 or the light chain variable region of the antibody produced by the hybridoma cell line having an ATCC Accession Number HB-12109), by at least one, two, three, four, five, six, seven positions selected from the group consisting of:

a position within or adjacent to one or more of residues 5, 6, 7, 8, 9, 10, 11, 12, 13, 14, 15, 16, 17 or 18, or a T cell epitope which includes one or more of residues 5-18 (linear numbering as in FIG. 6);

a position within or adjacent to one or more of residues 11, 12, 13, 14, 15, 16, 17, 18, 19, 20, 21, 22, 23, or 24, or a T cell epitope which includes one or more of residues 11-24 (linear numbering as in FIG. 6);

a position within or adjacent to one or more of residues 13, 14, 15, 16, 17, 18, 19, 20, 21, 22, 23, 24, 25, or 26, or a T cell epitope which includes one or more of residues 13-26 (linear numbering as in FIG. 6);

a position within or adjacent to one or more of residues 17, 18, 19, 20, 21, 22, 23, 24, 25, 26, 27, 28, 29, or 30, or a T cell epitope which includes one or more of residues 17-30 (linear numbering as in FIG. 6);

a position within or adjacent to one or more of residues 27, 28, 29, 30, 31, 32, 33, 34, 35, 36, 37, 38, 39, or 40, or a T cell epitope which includes one or more of residues 27-40 (linear numbering as in FIG. 6);

a position within or adjacent to one or more of residues 31, 32, 33, 34, 35, 36, 37, 38, 39, 40, 41, 42, 43, or 44, or a T cell epitope which includes one or more of residues 31-44 (linear numbering as in FIG. 6);

a position within or adjacent to one or more of residues 56, 57, 58, 59, 60, 61, 62, 63, 64, 65, 66, 67, 68, or 69, or a T cell epitope which includes one or more of residues 56-69 (linear numbering as in FIG. 6);

a position within or adjacent to one or more of residues 60, 61, 62, 63, 64, 65, 66, 67, 68, 69, 70, 71, 72, or 73, or a T cell epitope which includes one or more of residues 60-73 (linear numbering as in FIG. 6);

a position within or adjacent to one or more of residues 70, 71, 72, 73, 74, 75, 76, 77, 78, 79, 80, 81, 82, or 83, or a T cell epitope which includes one or more of residues 70-83 (linear numbering as in FIG. 6);

a position within or adjacent to one or more of residues 71, 72, 73, 74, 75, 76, 77, 78, 79, 80, 81, 82, 83 or 84, or a T cell epitope which includes one or more of residues 71-84 (linear numbering as in FIG. 6);

a position within or adjacent to one or more of residues 73, 74, 75, 76, 77, 78, 79, 80, 81, 82, 83, 84, 85 or 86, or a T cell epitope which includes one or more of residues 73-86 (linear numbering as in FIG. 6);

a position within or adjacent to one or more of residues 76, 77, 78, 79, 80, 81, 82, 83, 84, 85, 86, 87, 88, 89, 90, 91, or 92, or a T cell epitope which includes one or more of residues 76-92 (linear numbering as in FIG. 6); and

a position within or adjacent to one or more of residues 81, 82, 83, 84, 85, 86, 87, 88, 89, 90, 91, 92, 93, or 94, or a T cell epitope which includes one or more of residues 81-94 (linear numbering as in FIG. 6).

In other embodiments, the heavy chain immunoglobulin of the anti-PSMA antibody, or antigen-binding fragment thereof, includes a non-human heavy chain variable region comprising three CDRs from a non-human, e.g., murine, anti-PSMA heavy chain variable region (e.g., the murine J591 heavy chain variable region shown in SEQ ID NO:19 (see FIG. 1A), or the heavy chain variable region of the antibody produced by the hybridoma cell line having an ATCC Accession Number HB-12126) and a heavy chain framework which differs from the framework of the non-human anti-PSMA heavy chain variable region (e.g., the murine J591 heavy chain variable region of SEQ ID NO:19 or the heavy chain variable framework of the antibody produced by the hybridoma cell line having an ATCC Accession Number HB-12126), by at least one, two, three, five, seven, ten positions selected from the group consisting of:

a position within or adjacent to one or more of residues 2, 3, 4, 5, 6, 7, 8, 9, 10, 11, 12, 13, or 14, or a T cell epitope which includes one or more of residues 2-14 (numbering as in FIG. 3A);

a position within or adjacent to one or more of residues 10, 11, 12, 13, 14, 15, 16, 17, 18, 19, 20, 21, or 22, or a T cell epitope which includes one or more of residues 10-22 (numbering as in FIG. 3A);

a position within or adjacent to one or more of residues 16, 17, 18, 19, 20, 21, 22, 23, 24, 25, 26, 27, or 28, or a T cell epitope which includes one or more of residues 16-28 (numbering as in FIG. 3A);

a position within or adjacent to one or more of residues 30, 31, 32, 33, 34, 35, 36, 37, 38, 39, 40, 41, or 42, or a T cell epitope which includes one or more of residues 30-42 (numbering as in FIG. 3A);

a position within or adjacent to one or more of residues 32, 33, 34, 35, 36, 37, 38, 39, 40, 41, 42, 43, or 44, or a T cell epitope which includes one or more of residues 32-44 (numbering as in FIG. 3A);

a position within or adjacent to one or more of residues 43, 44, 45, 46, 47, 48, 49, 50, 51, 52, 53, 54, or 55, or a T cell epitope which includes one or more of residues 43-55 (numbering as in FIG. 3A);

a position within or adjacent to one or more of residues 46, 47, 48, 49, 50, 51, 52, 53, 54, 55, 56, 57, or 58, or a T cell epitope which includes one or more of residues 46-58 (numbering as in FIG. 3A);

a position within or adjacent to one or more of residues 58, 59, 60, 61, 62, 63, 64, 65, 66, 67, 68, 69, or 70, or a T cell epitope which includes one or more of residues 58-70 (numbering as in FIG. 3A);

a position within or adjacent to one or more of residues 62, 63, 64, 65, 66, 67, 68, 69, 70, 71, 72, 73 or 74, or a T cell epitope which includes one or more of residues 62-74 (numbering as in FIG. 3A);

a position within or adjacent to one or more of residues 70, 71, 72, 73, 74, 75, 76, 77, 78, 79, 80 or 81, or a T cell epitope which includes one or more of residues 70-81 (numbering as in FIG. 3A);

a position within or adjacent to one or more of residues 81, 82, 83, 84, 85, 86, 87, 88, 89, 90, 91, 92, or 93, or a T cell epitope which includes one or more of residues 81-93 (numbering as in FIG. 3A);

a position within or adjacent to one or more of residues 84, 85, 86, 87, 88, 89, 90, 91, 92, 93, 95, or 96, or a T cell epitope which includes one or more of residues 84-96 (numbering as in FIG. 3A);

a position within or adjacent to one or more of residues 91, 92, 93, 95, 96, 97, 98, 99, 100, 101, 102, or 103, or a T cell epitope which includes one or more of residues 91-103 (numbering as in FIG. 3A); and

a position within or adjacent to one or more of residues 100, 101, 102, 103, 104, 105, 106, 107, 108, 109, 110, 111, or 112, or a T cell epitope which includes one or more of residues 100-112 (numbering as in FIG. 3A).

In other embodiments, the heavy chain immunoglobulin of the anti-PSMA antibody, or antigen-binding fragment thereof, includes a non-human heavy chain variable region comprising three CDRs from a non-human, e.g., murine, anti-PSMA heavy chain variable region (e.g., the murine J415 heavy chain variable region shown in SEQ ID NO:47 (see FIG. 5), or the heavy chain variable region of the antibody produced by the hybridoma cell line having an ATCC Accession Number HB-12109) and a heavy chain framework which differs from the framework of the non-human anti-PSMA heavy chain variable region, e.g., the murine J415 heavy chain variable region of SEQ ID NO:47 or the heavy chain variable framework of the antibody produced by the hybridoma cell line having an ATCC Accession Number HB-12109), by at least one, two, three, four, five positions selected from the group consisting of:

a position within or adjacent to one or more of residues 10, 11, 12, 13, 14, 15, 16, 17, 18, 19, 20, 21, 22 or 23, or a T cell epitope which includes one or more of residues 10-23 (numbering as in FIG. 5);

a position within or adjacent to one or more of residues 16, 17, 18, 19, 20, 21, 22, 23, 24, 25, 26, 27, 28, or 29, or a T cell epitope which includes one or more of residues 16-29 (numbering as in FIG. 5);

a position within or adjacent to one or more of residues 21, 22, 23, 24, 25, 26, 27, 28, 29, 30, 31, 32, 33, or 34, or a T cell epitope which includes one or more of residues 21-34 (numbering as in FIG. 5);

a position within or adjacent to one or more of residues 30, 31, 32, 33, 34, 35, 36, 37, 38, 39, 40, 41, 42, or 43, or a T cell epitope which includes one or more of residues 30-43 (numbering as in FIG. 5);

a position within or adjacent to one or more of residues 35, 36, 37, 38, 39, 40, 41, 42, 43, 44, 45, 46, 47, or 48, or a T cell epitope which includes one or more of residues 35-48 (numbering as in FIG. 5);

a position within or adjacent to one or more of residues 43, 44, 45, 46, 47, 48, 49, 50, 51, 52, 53, 54, 55, or 56, or a T cell epitope which includes one or more of residues 43-56 (numbering as in FIG. 5);

a position within or adjacent to one or more of residues 46, 47, 48, 49, 50, 51, 52, 53, 54, 55, 56, 57, 58 or 59, or a T cell epitope which includes one or more of residues 46-59 (numbering as in FIG. 5);

a position within or adjacent to one or more of residues 49, 50, 51, 52, 53, 54, 55, 56, 57, 58, 59, 60, 61, or 62, or a T cell epitope which includes one or more of residues 49-62 (numbering as in FIG. 5);

a position within or adjacent to one or more of residues 64, 65, 66, 67, 68, 69, 70, 71, 72, 73, 74, 75, 76, or 77, or a T cell epitope which includes one or more of residues 64-77 (numbering as in FIG. 5);

a position within or adjacent to one or more of residues 80, 81, 82, 83, 84, 85, 86, 87, 88, 89, 90, 91, 92, or 93, or a T cell epitope which includes one or more of residues 80-93 (numbering as in FIG. 5);

a position within or adjacent to one or more of residues 86, 87, 88, 89, 90, 91, 92, 93, 94, 95, 96, 97, 98, or 99, or a T cell epitope which includes one or more of residues 86-99 (numbering as in FIG. 5); and

a position within or adjacent to one or more of residues 104, 105, 106, 107, 108, 109, 110, 111, 112, 113, 114, 115, 116, or 117, or a T cell epitope which includes one or more of residues 104-117 (numbering as in FIG. 5).

In yet other embodiments, the anti-PSMA antibody, or antigen-binding fragment thereof, includes at least one light or heavy chain immunoglobulin or, more preferably, at least one light chain immunoglobulin and at least one heavy chain immunoglobulin. Preferably, the light chain immunoglobulin includes a non-human light chain variable region comprising three CDRs from a non-human, e.g., murine, anti-PSMA light chain variable region (e.g., the murine J591 light chain variable region shown in SEQ ID NO:20 (FIG. 1B), or the light chain variable region of the antibody produced by the hybridoma cell line having an ATCC Accession Number HB-12126) and a light chain framework which differs from the framework of the non-human anti-PSMA light chain variable region, e.g., murine J591 light chain variable region, by at least one position while having a residue from the non-human anti-PSMA light chain variable region at least one, two, three, five, seven, ten, fifteen, or twenty residues selected from the group consisting of 1, 2, 4-7, 12-19, 23, 31-41, 43-49, 57, 59, 61, 62, 64-75, 79, 82, 83, 85-87, 89, 98, 99, 101, 102, 105, and 106 (numbering as in FIG. 3B). The light chain framework can differ at a positions chosen from one, two, three, four, five, six, seven, eight, nine, ten, eleven, twelve, thirteen, fourteen, fifteen, sixteen, nineteen, twenty or more residues selected from the group consisting of 3, 8, 9, 10, 11, 20, 21, 22, 42, 58, 60, 63, 76, 77, 78, 80, 83, 87, 100, 103, and 104 (numbering as in FIG. 3B).

In yet other embodiments, the anti-PSMA antibody, or antigen-binding fragment thereof, includes at least one light or heavy chain immunoglobulin or, more preferably, at least one light chain immunoglobulin and at least one heavy chain immunoglobulin. Preferably, the modified light chain immunoglobulin includes a non-human light chain variable region comprising three CDRs from a non-human, e.g., murine, anti-PSMA light chain variable region (e.g., the murine J415 light chain variable region shown in SEQ ID NO:48 (FIG. 6), or the light chain variable region of the antibody produced by the hybridoma cell line having an ATCC Accession Number HB-12109) and a light chain framework which differs from the framework of the non-human anti-PSMA light chain variable region, e.g., murine J415 light chain variable region, by at least one position while having a residue from the non-human anti-PSMA light chain variable region at least one, two, three, five, seven, ten, fifteen, or twenty residues selected from the group consisting of 1-12, 14, 16-18, 20-40, 42-62, 64-67, 69-79, and 81-107 (linear numbering as in FIG. 6). The modified light chain framework can differ at least one, two, three, four, five, six, or seven positions selected from the group consisting of 13, 15, 19, 41, 63, 68 and 80 (linear numbering as in FIG. 6).

In other embodiments, the heavy chain immunoglobulin of the modified anti-PSMA antibody, or antigen-binding fragment thereof, includes a non-human heavy chain variable region comprising three CDRs from a non-human, e.g., murine, anti-PSMA heavy chain variable region (e.g., the murine J591 heavy chain variable region shown in SEQ ID NO:19 (FIG. 1A), or the heavy chain variable region of the antibody produced by the hybridoma cell line having an ATCC Accession Number HB-12126) and a modified heavy chain framework which differs from the framework of the non-human anti-PSMA heavy chain variable region by at least one position while having a residue from the non-human anti-PSMA heavy chain variable region at least one, two, three, four, five, six, seven, eight, nine, ten, eleven, twelve, thirteen, or fourteen residues selected from the group consisting of 1-4,6-10, 13-15, 18, 20-25, 36-39, 42, 43, 45-49, 67-75, 78-83, 85, 86, 88-90, 92-98, 105-109, and 111-115 (numbering as in FIG. 3A). The modified heavy chain framework can differ at at least one, two, three, four, five, six, seven, eight, nine, ten, eleven, twelve, thirteen, or fourteen positions selected from the group consisting of 5, 11-12, 16-17, 19, 26-35, 40-41, 44, 50-66, 76-77, 84, 87, 91, 99-104, and 110 (numbering as in FIG. 3A).

In other embodiments, the heavy chain immunoglobulin of the anti-PSMA antibody, or antigen-binding fragment thereof, includes a non-human heavy chain variable region comprising three CDRs from a non-human, e.g., murine, anti-PSMA heavy chain variable region (e.g., the murine J415 heavy chain variable region shown in SEQ ID NO:47 (FIG. 5), or the heavy chain variable region of the antibody produced by the hybridoma cell line having an ATCC Accession Number HB-12109) and a heavy chain framework which differs from the framework of the non-human anti-PSMA heavy chain variable region by at least one position while having a residue from the non-human anti-PSMA heavy chain variable region at at least one, two, three, four, or five residues selected from the group consisting of 1-19, 21-86, 88-93, 96-111, and 113-116 (numbering as in FIG. 5). The heavy chain framework can differ at a positions selected from the group consisting of 20, 87, 94, 95 and 112 (numbering as in FIG. 5).

In yet another aspect, the heavy chain immunoglobulin of the anti-PSMA antibody, or antigen-binding fragment thereof, includes a heavy chain variable region comprising at least one, two, three, four, five, six, seven, eight, nine, ten, twenty, twenty-five, thirty, thirty-five, forty, forty-five, or fifty amino acid residues chosen from one or more of the following residues and located at a position chosen from one or more of: residue 1 (glutamate), 2 (valine), 4 (leucine), 7 (serine), 8 (glycine), 11 (leucine), 14 (proline), 15 (glycine), 19 (lysine), 20 (isoleucine), 21 (serine), 22 (cysteine), 25 (serine), 26 (glycine), 28 (threonine), 29 (phenylalanine), 32 (tyrosine), 36 (tryptophan), 37 (valine), 38 (arginine/lysine), 39 (glutamine), 41 (proline), 43 (lysine), 44 (glycine), 45 (leucine), 46 (glutamate), 47 (tryptophan), 51 (isoleucine), 67 (arginine/lysine), 73 (aspartate), 75 (serine), 80 (tyrosine), 85 (serine), 86 (leucine), 87 (arginine), 89 (glutamate), 90 (aspartate), 91 (threonine), 92 (alanine), 93 (valine), 94 (tyrosine), 95 (tyrosine), 96 (cysteine), 100 (tryptophan), 101 (asparagine), 105 (tryptophan), 106 (glycine), 107 (glutamine), 108 (glycine), 109 (threonine), 112 (threonine), 113 (valine), 114 (serine), or 115 (serine) (linear numbering as shown in FIG. 3A).

In one embodiment, the heavy chain immunoglobulin of the anti-PSMA antibody, or antigen-binding fragment thereof, includes one or more of:

a framework region 1 having at least one, two, three, four, five, six, seven, eight, nine, ten, eleven, twelve, thirteen amino acids selected from the group consisting of residue 1 (glutamate), 2 (valine), 4 (leucine), 7 (serine), 8 (glycine), 11 (leucine), 14 (proline), 15 (glycine), 19 (lysine), 20 (isoleucine), 21 (serine), 22 (cysteine), and 25 (serine) (linear numbering as shown in FIG. 3A);

a CDR1 having at least one, two, three, four amino acids selected from the group consisting of residue 26 (glycine), 28 (threonine), 29 (phenylalanine), and 32 (tyrosine) (linear numbering as shown in FIG. 3A);

a framework region 2 having at least one, two, three, four, five, six, seven, eight, nine, ten amino acids selected from the group consisting of residue 36 (tryptophan), 37 (valine), 38 (arginine/lysine), 39 (glutamine), 41 (proline), 43 (lysine), 44 (glycine), 45 (leucine), 46 (glutamate), and 47 (tryptophan) (linear numbering as shown in FIG. 3A);

a CDR2 having at least one isoleucine at position 51 (linear numbering as shown in FIG. 3A);

a framework region 3 having at least one, two, three, four, five, six, seven, eight, nine, ten, eleven, twelve, thirteen, fourteen amino acids selected from the group consisting of residue 67 (arginine/lysine), 73 (aspartate), 75 (serine), 80 (tyrosine), 85 (serine), 86 (leucine), 87 (arginine), 89 (glutamate), 90 (aspartate), 91 (threonine), 92 (alanine), 93 (valine), 94 (tyrosine), 95 (tyrosine), and 96 (cysteine) (linear numbering as shown in FIG. 3A);

a CDR3 having at least one, two amino acids selected from the group consisting of residue 100 (tryptophan) and 101 (asparagine) (linear numbering as shown in FIG. 3A); or

a framework region 4 having at least one, two, three, four, five, six, seven, eight, nine amino acids selected from the group consisting of residue 105 (tryptophan), 106-(glycine), 107 (glutamine), 108 (glycine), 1-09 (threonine), 112 (threonine), 113 (valine), 114 (serine), and 115 (serine) (linear numbering as shown in FIG. 3A).

In yet another embodiment, the light chain immunoglobulin of the modified anti-PSMA antibody, or antigen-binding fragment thereof, includes a light chain variable region comprising at least one, two, three, four, five, six, seven, eight, nine, ten, eleven, twelve, thirteen, fourteen, twenty, thirty, forty, fifty, sixty, or seventy amino acids chosen from one or more of the following residues and located at a position chosen from one or more of: residue 2 (isoleucine), 4 (methionine), 5 (threonine), 6 (glutamine), 8 (proline), 10 (serine), 12 (serine), 14 (serine), 16 (glycine), 17 (glutamate/aspartate), 18 (arginine), 20 (threonine), 21 (leucine), 22 (threonine), 23 (cysteine), 24 (lysine), 25 (alanine), 26 (serine), 29 (valine), 30 (glycine), 31 (threonine), 33 (valine), 35 (tryptophan), 36 (tyrosine), 37 (glutamine), 38 (glutamine), 39 (lysine), 40 (proline), 43 (serine), 44 (proline), 45 (lysine), 47 (leucine), 48 (isoleucine), 49 (tyrosine), 51 (alanine), 52 (serine), 54 (arginine), 56 (threonine), 57 (glycine), 59 (proline), 61 (arginine), 62 (phenylalanine), 63 (serine), 64 (glycine), 65 (serine), 66 (glycine), 67 (serine), 68 (glycine), 69 (threonine), 70 (aspartate), 71 (phenylalanine), 73 (leucine), 74 (threonine), 75 (threonine), 76 (serine), 77 (serine), 79 (glutamine), 81 (glutamate), 82 (aspartate), 85 (aspartate), 86 (tyrosine), 87 (tyrosine), 88 (cysteine), 90 (glutamine), 95 (proline), 97 (threonine), 98 (phenylalanine), 99 (glycine), 101 (glycine), 102 (threonine), 103 (lysine), 105 (glutamate/aspartate), or 107 (lysine) (linear numbering as in FIG. 3B).

In one embodiment, the light chain immunoglobulin of the anti-PSMA antibody, or antigen-binding fragment thereof, includes one or more of:

a framework region 1 having at least one, two, three, four, five, six, seven, eight, nine, ten, eleven, twelve, fourteen, fifteen amino acids selected from the group consisting of residue 2 (isoleucine), 4 (methionine), 5 (threonine), 6 (glutamine), 8 (pro line), 10 (serine), 12 (serine), 14 (serine), 16 (glycine), 17 (glutamate/aspartate), 18 (arginine), 20 (threonine), 21 (leucine), 22 (threonine), and 23 (cysteine) (linear numbering as shown in FIG. 3B);

a CDR1 having at least one, two, three, four, five, six, seven amino acids selected from the group consisting of residue 24 (lysine), 25 (alanine), 26 (serine), 29 (valine), 30 (glycine), 31 (threonine), and 33 (valine) (linear numbering as shown in FIG. 3B);

a framework region 2 having at least one, two, three, four, five, six, seven, eight, nine, ten, eleven, twelve amino acids selected from the group consisting of residue 35 (tryptophan), 36 (tyrosine), 37 (glutamine), 38 (glutamine), 39 (lysine), 40 (proline), 43 (serine), 44 (proline), 45 (lysine), 47 (leucine), 48 (isoleucine), and 49 (tyrosine) (linear numbering as shown in FIG. 3B);

a CDR2 having at least one, two, three, four amino acids selected from the group consisting of residue 51 (alanine), 52 (serine), 54 (arginine), and 56 (threonine) (linear numbering as shown in FIG. 3B);

a framework region 3 having at least one, two, three, four, five, six, seven, eight, nine, ten, eleven, twelve, thirteen, fourteen, fifteen, twenty, twenty-one, twenty-two, twenty-three, twenty-four amino acids selected from the group consisting of residue 59 (proline), 61 (arginine), 62 (phenylalanine), 63 (serine), 64 (glycine), 65 (serine), 66 (glycine), 67 (serine), 68 (glycine), 69 (threonine), 70 (aspartate), 71 (phenylalanine), 73 (leucine), 74 (threonine), 75 (threonine), 76 (serine), 77 (serine), 79 (glutamine), 81 (glutamate), 82 (aspartate), 85 (aspartate), 86 (tyrosine), 87 (tyrosine), and 88 (cysteine) (linear numbering as shown in FIG. 3B);

a CDR3 having at least one, two, three, four amino acids selected from the group consisting of residue 90 (glutamine), 95 (proline), 97 (threonine), and 98 (phenylalanine) (linear numbering as shown in FIG. 4B); or a framework region 4 having at least one, two, three, four, five, six amino acid selected from the group consisting of residue 99 (glycine), 101 (glycine), 102 (threonine), 103 (lysine), 105 (glutamate/aspartate), and 107 (lysine) (linear numbering as shown in FIG. 3B).

Other Methods of Producing Anti-PSMA Antibodies

Monoclonal anti-PSMA antibodies can also be generated by other methods known to those skilled in the art of recombinant DNA technology.

As used herein, “an in vitro generated” “antibody” or “immunoglobulin” refers to an immunoglobulin where all or part of the variable region, e.g., one or more or all CDRs, is generated in a non-immune cell selection, e.g., an in vitro phage-display, protein chip- or any other method in which candidate sequences can be tested for their ability to bind to an antigen.

An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, BioTechniques 11: 152-156). A similar strategy can also be used to amplify human heavy and light chain variable regions from human antibodies (Larrick et al., 1991, Methods: Companion to Methods in Enzymology 2:106-110).

In an illustrative embodiment, RNA is isolated from B lymphocytes, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combination, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFv gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

Once displayed on the surface of a display package (e.g., filamentous phage), the antibody library is screened with the antigen, or peptide fragment thereof, to identify and isolate packages that express an antibody having specificity for the antigen. Nucleic acid encoding the selected antibody can be recovered from the display package (e.g., from the phage genome) and subcloned into other expression vectors by standard recombinant DNA techniques.

Specific antibodies with high affinities for a surface protein can be made according to methods known to those in the art, e.g., methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies.

In particular, the Fv binding surface of a particular antibody molecule interacts with its target ligand according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence information, by a modeling procedure using previously determined three-dimensional structures from other antibodies obtained from NMR studies or crystallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol. 51, op. cit., pp 1-15.

An antigen-binding region can also be obtained by screening various types of combinatorial libraries with a desired binding activity, and to identify the active species, by methods that have been described.

In one embodiment, a variegated peptide library is expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are described above.

Other techniques include affinity chromatography with an appropriate “receptor” to isolate binding agents, followed by identification of the isolated binding agents or ligands by conventional techniques (e.g., mass spectrometry and NMR). Preferably, the soluble receptor is conjugated to a label (e.g., fluorophores, colorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library (see, e.g., W. C. Still et al., International Application WO 94/08051). In general, this method features the use of inert but readily detectable tags, that are attached to the solid support or to the compounds. When an active compound is detected, the identity of the compound is determined by identification of the unique accompanying tag. This tagging method permits the synthesis of large libraries of compounds which can be identified at very low levels among to total set of all compounds in the library.

Anti-PSMA antibody that are not intact antibodies are also useful in this invention. Such antibodies may be derived from any of the antibodies described above. For example, antigen-binding fragments, as well as full-length monomeric, dimeric or trimeric polypeptides derived from the above-described antibodies are themselves useful. Useful antibody homologs of this type include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

Antibody fragments may also be produced by chemical methods, e.g., by cleaving an intact antibody with a protease, such as pepsin or papain, and optionally treating the cleaved product with a reducing agent. Alternatively, useful fragments may be produced by using host cells transformed with truncated heavy and/or light chain genes.

Monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

In one embodiment, the constant region of the antibody can be replaced by another constant region from, e.g., a different species. This replacement can be carried out using molecular biology techniques. For example, the nucleic acid encoding the VL or VH region of a antibody can be converted to a full-length light or heavy chain gene, respectively, by operatively linking the VH or VL-encoding nucleic acid to another nucleic acid encoding the light or heavy chain constant regions. The sequences of human light and heavy chain constant region genes are known in the art. Preferably, the constant region is human, but constant regions from other species, e.g., rodent (e.g., mouse or rat), primate, camel, rabbit, can also be used. Constant regions from these species are known in the art (see e.g., Kabat, E. A., et al. (1991) Sequences of Proteins of Immunological Interest, Fifth Edition, U.S. Department of Health and Human Services, NIH Publication No. 91-3242).

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described, which if applied to immunoglobulins of murine or other species, would reduce or eliminate these functions.

An anti-PSMA antibody, or antigen-binding fragment thereof, can be derivatized or linked to another functional molecule (e.g., another peptide or protein). Accordingly, the antibodies and antibody portions of the invention are intended to include derivatized and otherwise modified forms of the antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized (or labeled) to include fluorescent compounds, various enzymes, prosthetic groups, luminescent materials, bioluminescent materials, fluorescent emitting metal atoms, e.g., europium (Eu), and other lanthanides, and radioactive materials (described below). Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibodies can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An anti-PSMA antibody or antigen-binding fragment thereof may be conjugated to a another molecular entity, typically a label or a therapeutic (e.g., a cytotoxic or cytostatic) agent or moiety.

Radioactive isotopes can be used in diagnostic or therapeutic applications. Radioactive isotopes that can be coupled to the anti-PSMA antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹² Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹ Cr), chlorine (³⁶Cl), cobalt (⁵⁷Co or ⁵¹Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹ At), rhenium (¹¹⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The anti-PSMA antibodies can be radiolabeled using techniques known in the art. For example, the method includes contacting an anti-PSMA antibody, e.g. an anti-PSMA antibody described herein, with a chelating agent, e.g., 1,4,7,10-tetraazacyclododecane-N,N′,N″,N′″-tetraacetic acid (DOTA), to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled anti-PSMA antibody. Detailed procedures for radiolabeling an anti-PSMA antibody are described in more detail in the sections below and the appended examples. For example, the anti-PSMA antibodies can be radiolabeled with ¹¹¹Indium, ⁹⁰Yttrium and ¹⁷⁷Lutetium by coupling with 1,4,7,10-tetraazacyclododecane-N,N′,N″,N′″-tetraacetic acid (DOTA) as described in U.S. S. No. 60/295,214, filed on Jun. 1, 2001, the contents of which are incorporated by reference in its entirety. Detailed experimental protocols for chelating anti-PSMA antibodies are described in Example 15 of U.S. S No. 60/295,214, which is specifically incorporated by reference in the present application and is reproduced below in the examples.

As is discussed above, the antibody can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol or DM1 (see U.S. Pat. No. 5,208,020), CC-1065 (see U.S. Pat. Nos. 5,475,092, 5,585,499, 5,846,545) calicheamicin, and analogs or homologs thereof. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. No. 6,333,410, the contents of which is incorporated herein by reference. The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include α₁-BR, α₂-BR, α₃-BR, α₄-BR, β₁-BR, β₂-BR and γ₁-BR. Iodo-complex calicheamicins include α₁-I, α₂-I, α₃-I, β₁-I, β₂-I, δ₁-I and γ₁-BR. Calicheamicin and mutants, analogs and mimics thereof are described, for example, in U.S. Pat. No. 4,970,198, issued Nov. 13, 1990, U.S. Pat. No. 5,264,586, issued Nov. 23, 1993, U.S. Pat. No. 5,550,246, issued Aug. 27, 1996, U.S. Pat. No. 5,712,374, issued Jan. 27, 1998, and U.S. Pat. No. 5,714,586, issued Feb. 3, 1998, the contents of which are incorporated herein by reference. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

The conjugates of the invention can be used for modifying a given biological response. The therapeutic agent is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Nucleic Acids, Vectors and Host Cells

Isolated nucleic acid, vector and host cell compositions that can be used for recombinant expression of the modified antibodies and antigen-binding fragments of the invention are disclosed. In one embodiment, a first and second isolated nucleic acid comprising a nucleotide sequence encoding heavy and light chain variable regions, respectively, of an anti-PSMA antibody, e.g., a modified anti-PSMA antibody (e.g., a deimmunized J591 or J415 anti-PSMA antibody), or an antigen fragment thereof, are provided.

The nucleotide and amino acid sequence of the modified (deimmunized) anti-PSMA J591 immunoglobulin light chain variable region is shown in FIG. 4B (SEQ ID NO:25 and 22, respectively). The non-coding complementary nucleotide sequence is also shown in FIG. 4B (SEQ ID NO:26). The J591 deimmunized anti-PSMA antibody light chain variable region contains the following regions: an FR1 domain corresponding to about amino acid residues 1-23 of SEQ ID NO:22 (linear numbering; see also SEQ ID NO:13), which is encoded by about nucleotides 261-329 of SEQ ID NO:25; a CDR1 domain corresponding to about amino acid residues 24-34 of SEQ ID NO:22 (linear numbering; see also SEQ ID NO:4), which is encoded by about nucleotides 330-362 of SEQ ID NO:25; an FR2 domain corresponding to about amino acid residues 35-49 of SEQ ID NO:22 (linear numbering; see also SEQ ID NO:14), which is encoded by about nucleotides 363-407 of SEQ ID NO:25; a CDR2 domain corresponding to about amino acid residues 50-56 of SEQ ID NO:22 (linear numbering; see SEQ ID NO:5), which is encoded by about nucleotides 408-428 of SEQ ID NO:25; an FR3 domain corresponding to about amino acid residues 57-88 of SEQ ID NO:22 (linear numbering; see also SEQ ID NO:15), which is encoded by about nucleotides 429-524 of SEQ ID NO:25; a CDR3 domain corresponding to about amino acid residues 89-97 of SEQ ID NO:22 (linear numbering; see also SEQ ID NO:6), which is encoded by about nucleotides 525-551 of SEQ ID NO:25; and an FR4 domain corresponding to about amino acid residues 98-107 of SEQ ID NO:22 (linear numbering; see also SEQ ID NO:16), which is encoded by about nucleotides 552-581 of SEQ ID NO:25.

The nucleotide and amino acid sequence of the modified (deimmunized) anti-PSMA J591 immunoglobulin heavy chain variable region is shown in FIG. 4A (SEQ ID NO:23 and 21, respectively). The non-coding complementary sequence is also shown in FIG. 4A (SEQ ID NO:24). The J591 deimmunized anti-PSMA antibody heavy chain variable region contains the following regions: an FR1 domain corresponding to about amino acid residues 1-25 of SEQ ID NO:21 (linear numbering; see also SEQ ID NO:9), which is encoded by about nucleotides 261-335 of SEQ ID NO:23; a CDR1 domain corresponding to about amino acid residues 26-35 of SEQ ID NO:21 (linear numbering; see also SEQ ID NO:1), which is encoded by about nucleotides 336-365 of SEQ ID NO:23; an FR2 domain corresponding to about amino acid residues 36-49 of SEQ ID NO:21 (linear numbering; see also SEQ ID NO:10), which is encoded by about nucleotides 366-407 of SEQ ID NO:23; a CDR2 domain of corresponding to about amino acid residues 50-66 of SEQ ID NO:21 (linear numbering; see also SEQ ID NO:2), which is encoded by about nucleotides 408-458 of SEQ ID NO:23; an FR3 domain corresponding to about amino acid residues 67-98 of SEQ ID NO:21 (linear numbering; see also SEQ ID NO:11), which is encoded by about nucleotides 459-554 of SEQ ID NO:23; a CDR3 domain corresponding to about amino acid residues 99-104 of SEQ ID NO:21 (linear numbering; see also SEQ ID NO:3), which is encoded by about nucleotides 555-572 of SEQ ID NO:23; and an FR4 domain corresponding to about amino acid residues 105-115 of SEQ ID NO:21 (linear numbering; see also SEQ ID NO:9), which is encoded by about nucleotides 573-605 of SEQ ID NO:23.

The nucleotide and amino acid sequence of the modified (deimmunized) anti-PSMA J415 immunoglobulin light chain variable region (J415DIVK1) is shown in FIG. 7A (SEQ ID NO:56 and 57, respectively). The non-coding complementary nucleotide sequence of J415DIVK1 is also shown in FIG. 8A (SEQ ID NO:58). The J415 deimmunized anti-PSMA antibody light chain variable region contains the following regions: an FR1 domain corresponding to about amino acid residues 1-23 of SEQ ID NO:57 (linear numbering; see also SEQ ID NO:41), which is encoded by about nucleotides 261-329 of SEQ ID NO:56; a CDR1 domain corresponding to about amino acid residues 24-34 of SEQ ID NO:57 (linear numbering; see also SEQ ID NO:32), which is encoded by about nucleotides 330-362 of SEQ ID NO:56; an FR2 domain corresponding to about amino acid residues 35-49 of SEQ ID NO:57 (linear numbering; see also SEQ ID NO:42), which is encoded by about nucleotides 363-407 of SEQ ID NO:56; a CDR2 domain corresponding to about amino acid residues 50-56 of SEQ ID NO:57 (linear numbering; see also SEQ ID NO:33), which is encoded by about nucleotides 408-428 of SEQ ID NO:56; an FR3 domain corresponding to about amino acid residues 57-88 of SEQ ID NO:57 (linear numbering; see also SEQ ID NO:43), which is encoded by about nucleotides 429-524 of SEQ ID NO:56; a CDR3 domain corresponding to about amino acid residues 89-97 of SEQ ID NO:57 (linear numbering; see also SEQ ID NO:34), which is encoded by about nucleotides 525-551 of SEQ ID NO:56; and an FR4 domain corresponding to about amino acid residues 98-107 of SEQ ID NO:57 (linear numbering; see also SEQ ID NO:44), which is encoded by about nucleotides 552-581 of SEQ ID NO:56. The nucleotide and amino acid sequences of the preferred modified (deimmunized) anti-PSMA J415 immunoglobulin light chain variable region (J415DIVK5) are shown in SEQ ID NO:50 and 52, respectively; J415DIVK5 can be broken down into its component sequences in a manner identical to that shown above for J415DIVK1.

The nucleotide and amino acid sequence of the modified (deimmunized) anti-PSMA J415 immunoglobulin heavy chain variable region is shown in FIG. 7A (SEQ ID NO:53 and 54, respectively). The non-coding complementary sequence is also shown in FIG. 7A (SEQ ID NO:55). The J415 deimmunized anti-PSMA antibody heavy chain variable region contains the following regions: an FR1 domain corresponding to about amino acid residues 1-25 of SEQ ID NO:54 (linear numbering; see also SEQ ID NO:37), which is encoded by about nucleotides 261-335 of SEQ ID NO:53; a CDR1 domain corresponding to about amino acid residues 26-35 of SEQ ID NO:54 (linear numbering; see also SEQ ID NO:29), which is encoded by about nucleotides 336-365 of SEQ ID NO:53; an FR2 domain corresponding to about amino acid residues 36-49 of SEQ ID NO:54 (linear numbering; see also SEQ ID NO:38), which is encoded by about nucleotides 366-407 of SEQ ID NO:53; a CDR2 domain corresponding to about amino acid residues 50-68 of SEQ ID NO:54 (linear numbering; see also SEQ ID NO:30), which is encoded by about nucleotides 408-464 of SEQ ID NO:53; an FR3 domain corresponding to about amino acid residues 69-100 of SEQ ID NO:54 (linear numbering; see also SEQ ID NO:39), which is encoded by about nucleotides 465-560 of SEQ ID NO:53; a CDR3 domain corresponding to about amino acid residues 101-105 of SEQ ID NO:54 (linear numbering; see also SEQ ID NO:31), which is encoded by about nucleotides 561-575 of SEQ ID NO:53; and an FR4 domain corresponding to about amino acid residues 106-116 of SEQ ID NO:54 (linear numbering; see also SEQ ID NO:40), which is encoded by about nucleotides 576-608 of SEQ ID NO:53. The nucleotide and amino acid sequences of the preferred modified (deimmunized) anti-PSMA J415 immunoglobulin heavy chain variable region (J415DIVH4) are shown in SEQ ID NO:51 and 49, respectively; J415DIVH4 can be broken down into its component sequences in a manner identical to that shown above for J415DIVH1.

It will be appreciated by the skilled artisan that nucleotide sequences encoding anti-PSMA modified antibodies (e.g., FR domains, e.g., FR1-4), can be derived from the nucleotide and amino acid sequences described in the present application using the genetic code and standard molecular biology techniques.

In one embodiment, the isolated nucleic acid comprises an anti-PSMA modified antibody heavy chain variable region nucleotide sequence having a nucleotide sequence as shown in FIG. 4A (SEQ ID NO:23), FIG. 7A (SEQ ID NO:53) or SEQ ID NO:51 (for J415DIVH4) or a complement thereof (e.g., SEQ ID NO:24 or SEQ ID NO:55), the nucleotide sequence of the heavy chain variable region of the antibody produced by the NSO cell line having ATCC Accession Number PTA-3709 or PTA-4174 or a complement thereof; a sequence at least 85%, 90%, 95%, 99% or more identity thereto; or a sequence capable of hybridizing under stringent conditions described herein (e.g., highly stringent conditions) to a nucleotide sequence shown in FIG. 4A (SEQ ID NO:23), FIG. 7A (SEQ ID NO:53), SEQ ID NO:51, or a complement thereof (e.g., SEQ ID NO:24 or SEQ ID NO:55), or the nucleotide sequence of the heavy chain variable region of the antibody produced by the NSO cell line having ATCC Accession Number PTA-3709 or PTA-4174, or a complement thereof.

In another embodiment, the isolated nucleic acid encodes an anti-PSMA modified antibody heavy chain variable region amino acid sequence having an amino acid sequence as shown in FIG. 2A (SEQ ID NO:21) or FIG. 5 (e.g., SEQ ID NO:49), or the amino acid sequence of the heavy chain variable region of the antibody produced by the NSO cell line having ATCC Accession Number PTA-3709 or PTA-4174; a sequence at least 85%, 90%, 95%, 99% or more identical thereto; or a sequence capable of hybridizing under stringent conditions described herein (e.g., highly stringent conditions) to a nucleotide sequence encoding the amino acid sequence as shown in FIG. 2A (SEQ ID NO:21), FIG. 5 (e.g., SEQ ID NO:49), or the amino acid sequence of the heavy chain variable region of the antibody produced by the NSO cell line having ATCC Accession Number PTA-3709 or PTA-4174.

In another embodiment, the isolated nucleic acid comprises a nucleotide sequence encoding at least one, preferably two, and most preferably three, CDRs of the heavy chain variable region of the anti-PSMA antibody chosen from the amino acid sequences of SEQ ID NO:1, 2, and 3, or 29, 30 and 31, or 93, 94, and 95, or 99, 100 and 101, or a CDR sequence which differs by one or two amino acids from the sequences described herein. In yet another embodiment, the isolated nucleic acid comprises a nucleotide sequence encoding CDRs 1, 2, or 3 shown in FIG. 4A (SEQ ID NO:23), in SEQ ID NO:51, in FIG. 7A (SEQ ID NO:125), in FIG. 9A (SEQ ID NO:73), or in FIG. 11A (SEQ ID NO:83), or a complement thereof, or a sequence encoding a CDR that differs by one or two amino acids from the sequences described herein.

In another embodiment, the isolated nucleic acid comprises a nucleotide sequence encoding at least one, preferably two, three and most preferably four amino acid sequences from the heavy chain variable framework region of the anti-PSMA modified antibody chosen from SEQ ID NO:9, 10, 11 and 12, or 37, 38, 39 and 40, or a sequence at least 85%, 90%, 95%, 99% or more identical thereto.

In yet another embodiment, the isolated nucleic acid comprises an anti-PSMA modified antibody light chain variable region nucleotide sequence having a sequence as shown in FIG. 4B (SEQ ID NO:25), FIG. 8A (SEQ ID NO:56), or SEQ ID NO:52, or a complement thereof (e.g., SEQ ID NO:26 or 58), or the nucleotide sequence of the light chain variable region of the antibody produced by the NSO cell line having ATCC Accession Number PTA-3709 or PTA-4174; a sequence at least 85%, 90%, 95%, 99% or more identical thereto; or a sequence capable of hybridizing under stringent conditions described herein (e.g., highly stringent conditions) to the nucleotide sequence as shown in FIG. 4B (SEQ ID NO:25), FIG. 8A (SEQ ID NO:56), SEQ ID NO:52, or a complement thereof (e.g., SEQ ID NO:26 or 58), or the nucleotide sequence of the light chain variable region of the antibody produced by the NSO cell line having ATCC Accession Number PTA-3709 or PTA-4174, or a complement thereof. In another embodiment, the isolated nucleic acid encodes an anti-PSMA modified antibody light chain variable region amino acid sequence having a sequence as shown in FIG. 2B (SEQ ID NO:22) or in FIG. 6 (e.g., SEQ ID NO:50), the amino acid sequence of the light chain variable region of the antibody produced by the NSO cell line having ATCC Accession Number PTA-3709 or PTA-4174; a sequence at least 85%, 90%, 95%, 99% or more identity thereto; or a sequence capable of hybridizing under stringent conditions described herein (e.g., highly stringent conditions) to a nucleotide sequence encoding the amino acid sequence as shown in FIG. 2B (SEQ ID NO:22) or in FIG. 6 (SEQ ID NO:50), or the amino acid sequence of the light chain variable region of the antibody produced by the NSO cell line having ATCC Accession Number PTA-3709 or PTA-4174.

In another embodiment, the isolated nucleic acid comprises a nucleotide sequence encoding at least one, preferably two, and most preferably three, CDRs of the light chain variable region of the anti-PSMA antibody chosen from the amino acid sequences of SEQ ID NO:4, 5, and 6, or 32, 33, and 34, or 96, 97, and 98, or 102, 103, and 104, or a sequence encoding a CDR which differs by one or two amino acids from the sequences described herein.

In yet another embodiment, the isolated nucleic acid comprises a nucleotide sequence encoding CDRs 1-3 of the light chain variable nucleotide sequence shown in SEQ ID NO:25, or a sequence encoding a CDR which differs by one or two amino acids from the sequences described herein. In another embodiment, the isolated nucleic acid comprises a nucleotide sequence encoding at least one, preferably two, three and most preferably four amino acid sequences from the light chain variable framework region of the anti-PSMA modified antibody chosen from SEQ ID NO:13, 14, 15, and 16, or 41, 42, 43, and 44, or a sequence at least 85%, 90%, 95%, 99% or more identical thereto.

In a preferred embodiment, there is an isolated first and second nucleic acid which have nucleotide sequences encoding a light chain and the heavy chain variable regions of an anti-PSMA antibody, respectively, wherein each isolated nucleic acid has at least one, two, three, four, five and preferably all CDRs chosen from the amino acid sequences of SEQ ID NO:1, 2, 3, 4, 5, and 6, or 29, 30, 31, 32, 33 and 34, or 93, 94, 95, 96, 97, and 98, or 99, 100, 101, 102, 103, and 104, or sequence encoding a CDR which differs by one or two amino acids from the sequences described herein.

The nucleic acid can encode only the light chain or the heavy chain variable region, or can also encode an antibody light or heavy chain constant region, operatively linked to the corresponding variable region. In one embodiment, the light chain variable region is linked to a constant region chosen from a kappa or a lambda constant region. Preferably, the light chain constant region is from a lambda type (e.g., a human type lambda). In another embodiment, the heavy chain variable region is linked to a heavy chain constant region of an antibody isotype selected from the group consisting of IgG (e.g., IgG1, IgG2, IgG3, IgG4), IgM, IgA1, IgA2, IgD, and IgE. Preferably, the heavy chain constant region is from an IgG (e.g., an IgG1) isotype, e.g., a human IgG1.

Nucleic acids of the invention can be chosen for having codons, which are preferred, or non-preferred, for a particular expression system. For example, the nucleic acid can be one in which at least one codon, at preferably at least 10%, or 20% of the codons has been altered such that the sequence is optimized for expression in E. coli, yeast, human, insect, NSO, or CHO cells.

In a preferred embodiment, the nucleic acid differs (e.g., differs by substitution, insertion, or deletion) from that of the sequences provided, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences. The differences are, preferably, differences or changes at nucleotides encoding a non-essential residue(s) or a conservative substitution(s).

In one embodiment, the first and second nucleic acids are linked, e.g., contained in the same vector. In other embodiments, the first and second nucleic acids are unlinked, e.g., contained in different vectors.

In another aspect, the invention features host cells and vectors (e.g., recombinant expression vectors) containing the nucleic acids, e.g., the first and second nucleic acids, of the invention.

Prokaryotic or eukaryotic host cells may be used. The terms “host cell” and “recombinant host cell” are used interchangeably herein. Such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E. coli, or eukaryotic, e.g., insect cells, yeast, or preferably mammalian cells (e.g., cultured cell or a cell line). Other suitable host cells are known to those skilled in the art.

Preferred mammalian host cells for expressing the anti-PSMA antibodies, or antigen-binding fragments thereof, include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), lymphocytic cell lines, e.g., NS0 myeloma cells and SP2 cells, COS cells, and a cell from a transgenic animal, e.g., e.g., mammary epithelial cell.

In another aspect, the invention features a vector, e.g., a recombinant expression vector. The recombinant expression vectors of the invention can be designed for expression of the modified antibodies, or an antigen-binding fragment thereof, in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to an antibody encoded therein, usually to the constant region of the recombinant antibody.

In addition to the antibody chain genes, the recombinant expression vectors of the invention carry regulatory sequences that are operatively linked and control the expression of the antibody chain genes in a host cell.

In an exemplary system for recombinant expression of a modified antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

Other PSMA Binding Agents

Also useful in the methods of this invention are PSMA mimetic agents. These agents, which include peptides, semi-peptidic compounds or non-peptidic compounds (e.g., small organic molecules), are inhibitors of PSMA activity.

In preferred embodiments, the agent is a member of a combinatorial library, e.g., a peptide or organic combinatorial library, or a natural product library. In a preferred embodiment, a plurality of test compounds, e.g., library members, includes at least 10, 10², 10³, 10⁴, 10⁵, 10⁶, 10⁷, or 10⁸ compounds. In a preferred embodiment, the plurality of test compounds, e.g., library members, share a structural or functional characteristic.

In one embodiment, the invention provides libraries of PSMA binding agents. The synthesis of combinatorial libraries is well known in the art and has been reviewed (see, e.g., E. M. Gordon et al., J. Med. Chem. (1994) 37:1385-1401; DeWitt, S. H.; Czamik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

Libraries of compounds of the invention can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen-binding site. It will be appreciated that a wide variety of peptidic, peptidomimetic, or non-peptidic compounds can be readily generated in this way.

The “split-pool” strategy results in a library of peptides, e.g., inhibitors, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Libraries of compounds can be screened to determine whether any members of the library have a desired activity, and, if so, to identify the active species. Methods of screening combinatorial libraries have been described (see, e.g., Gordon et al., J. Med. Chem., supra). Soluble compound libraries can be screened by affinity chromatography with an appropriate receptor to isolate ligands for the receptor, followed by identification of the isolated ligands by conventional techniques (e.g., mass spectrometry, NMR, and the like). Immobilized compounds can be screened by contacting the compounds with a soluble receptor; preferably, the soluble receptor is conjugated to a label (e.g., fluorophores, colorimetric enzymes, radioisotopes, luminescent compounds, and the like) that can be detected to indicate ligand binding. Alternatively, immobilized compounds can be selectively released and allowed to diffuse through a membrane to interact with a receptor. Exemplary assays useful for screening the libraries of the invention are described below.

In one embodiment, compounds of the invention can be screened for the ability to interact with PSMA polypeptide by assaying the activity of each compound to bind directly to the polypeptide, e.g., by incubating the test compound with a PSMA polypeptide and a lysate, in one well of a multiwell plate, such as a standard 96-well microtiter plate. In this embodiment, the activity of each individual compound can be determined. A well or wells having no test compound can be used as a control. After incubation, the activity of each test compound can be determined by assaying each well. Thus, the activities of a plurality of test compounds can be determined in parallel.

In still another embodiment, large numbers of test compounds can be simultaneously tested for binding activity. For example, test compounds can be synthesized on solid resin beads in a “one bead-one compound” synthesis; the compounds can be immobilized on the resin support through a photolabile linker. A plurality of beads (e.g., as many as 100,000 beads or more) can then be combined with yeast cells and sprayed into a plurality of “nano-droplets”, in which each droplet includes a single bead (and, therefore, a single test compound). Exposure of the nano-droplets to UV light then results in cleavage of the compounds from the beads. It will be appreciated that this assay format allows the screening of large libraries of test compounds in a rapid format.

Combinatorial libraries of compounds can be synthesized with “tags” to encode the identity of each member of the library (see, e.g., W. C. Still et al., U.S. Pat. No. 5,565,324 and PCT Publication Nos. WO 94/08051 and WO 95/28640). In general, this method features the use of inert, but readily detectable, tags, that are attached to the solid support or to the compounds. When an active compound is detected (e.g., by one of the techniques described above), the identity of the compound is determined by identification of the unique accompanying tag. This tagging method permits the synthesis of large libraries of compounds which can be identified at very low levels. Such a tagging scheme can be useful, e.g., in the “nano-droplet” screening assay described above, to identify compounds released from the beads.

In preferred embodiments, the libraries of compounds of the invention contain at least 30 compounds, more preferably at least 100 compounds, and still more preferably at least 500 compounds. In preferred embodiments, the libraries of compounds of the invention contain fewer than 10⁹ compounds, more preferably fewer than 10⁸ compounds, and still more preferably fewer than 10⁷ compounds.

Pharmaceutical Compositions

In another aspect, the present invention provides compositions, e.g., pharmaceutically acceptable compositions, which include a PSMA binding agent described herein, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. The carrier is suitable for intravenous, intramuscular, subcutaneous, parenteral, spinal or epidermal administration (e.g., by injection or infusion). Depending on the route of administration, the active compound, i.e., the PSMA binding agent may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection (e.g., by needleless injection). In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The PSMA binding agents can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In some embodiments, pharmaceutical compositions of PSMA binding agents, alone or in combination with other agent, can be administered orally. Additionally, the compositions can be delivered parenterally. Parenteral therapy is typically intra-dermal, intra-articular, intramuscular or intravenous. Alternatively, an aerosol can be used topically. In general, the route of administration is oral, or parenteral.

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other implants, delivery systems, and modules are known to those skilled in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. In one embodiment, the anti-PSMA antibody is administered by intravenous infusion at a rate of less than 10 mg/min, preferably less than or equal to 5 mg/min to reach a dose of about 1 to 100 mg/m², preferably about 5 to 50 mg/m², about 7 to 25 mg/m², and more preferably, about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of a binding agent. A “therapeutically effective amount” refers to an amount effective, at dosages and for periods of time necessary, to achieve the desired therapeutic result. A therapeutically effective amount of the PSMA binding agent may vary according to factors such as the disease state, age, sex, and weight of the individual, and the ability of the antibody or antibody portion to elicit a desired response in the individual. A therapeutically effective amount is also one in which any toxic or detrimental effects of the PSMA binding agent is outweighed by the therapeutically beneficial effects. A “therapeutically effective dosage” preferably inhibits a measurable parameter, relative to untreated subjects. The ability of a compound to inhibit a measurable parameter, e.g., blood glucose levels, can be evaluated in an animal model system predictive of efficacy in human insulin-related disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Kits

Also within the scope of the invention are kits including the PSMA binding agents of the invention along with instructions on how to use the PSMA agent or the combination of such agents to treat, prevent or detect a disorder described herein, e.g., an insulin-related disorder. In some embodiments, the kit can include one or more other elements including: instructions for use; other reagents, e.g., a label, a therapeutic agent, or an agent useful for chelating, or otherwise coupling, an antibody to a label or therapeutic agent, or a radio protective composition; devices or other materials for preparing the antibody for administration; pharmaceutically acceptable carriers; and devices or other materials for administration to a subject. Instructions for use can include instructions for diagnostic applications of the PSMA binding agent, e.g., anti-PSMA antibodies (or antigen-binding fragment thereof), to detect PSMA, in vitro, e.g., in a sample, e.g., a biopsy or cells from a patient having an insulin-related disorder, or in vivo. The kit can also have instructions for detecting PSMA in a co-existing or precipitating condition, e.g., a condition which exacerbates or aggravates an insulin-related disorder, in order to evaluate the risk of developing an insulin-related disorder. The instructions can include instructions for therapeutic application including suggested dosages and/or modes of administration, e.g., in a patient with an insulin-related disorder, e.g., diabetes mellitus, e.g., Type 2 diabetes. Other instructions can include instructions on coupling of the antibody to a chelator, a label or a therapeutic agent, or for purification of a conjugated antibody, e.g., from unreacted conjugation components. As discussed above, the kit can include a label, e.g., any of the labels described herein. As discussed above, the kit can include a therapeutic agent, e.g., a therapeutic agent described herein. The kit can include a reagent useful for chelating or otherwise coupling a label or therapeutic agent to the antibody, e.g., a reagent discussed herein. For example, a macrocyclic chelating agent, preferably 1,4,7,10-tetraazacyclododecane-N,N′,N″,N′″-tetraacetic acid (DOTA), can be included. The DOTA can be supplied as a separate component or the DOTA (or other chelator or conjugating agent) can be supplied already coupled to the antibody. Additional coupling agents, e.g., an agent such as N-hydroxysuccinimide (NHS), can be supplied for coupling the chelator, e.g., DOTA, to the antibody. In some applications the antibody will be reacted with other components, e.g., a chelator or a label or therapeutic agent, e.g., a radioisotope, e.g., yttrium or lutetium. In such cases, the kit can include one or more of a reaction vessel to carry out the reaction or a separation device, e.g., a chromatographic column, for use in separating the finished product from starting materials or reaction intermediates.

The kit can further contain at least one additional reagent, such as a diagnostic or therapeutic agent, e.g., a diagnostic or therapeutic agent as described herein, and/or one or more additional PSMA binding agents, e.g., anti-PSMA antibodies (or fragments thereof), formulated as appropriate, in one or more separate pharmaceutical preparations.

The kit can further contain a radioprotectant. The radiolytic nature of isotopes, e.g., ⁹⁰Yttrium (⁹⁰Y) is known. In order to overcome this radiolysis, radioprotectants may be included, e.g., in the reaction buffer, as long as such radioprotectants are benign, meaning that they do not inhibit or otherwise adversely affect the labeling reaction, e.g., of an isotope, such as of ⁹⁰Y, to the antibody.

The formulation buffer of the present invention may include a radioprotectant such as human serum albumin (HSA) or ascorbate, which minimize radiolysis due to yttrium or other strong radionuclides. Other radioprotectants are known in the art and could also be used in the formulation buffer of the present invention, i.e., free radical scavengers (phenol, sulfites, glutathione, cysteine, gentisic acid, nicotinic acid, ascorbyl palmitate, HOP(:O)H₂I glycerol, sodium formaldehyde sulfoxylate, Na₂S₂O, Na₂S₂O₃, and SO₂, etc.). A preferred kit is one useful for radiolabeling a chelator-conjugated protein or peptide with a therapeutic radioisotope for administration to a patient. The kit includes (i) a vial containing chelator-conjugated antibody, (ii) a vial containing formulation buffer for stabilizing and administering the radiolabeled antibody to a patient, and (iii) instructions for performing the radiolabeling procedure. The kit provides for exposing a chelator-conjugated antibody to the radioisotope or a salt thereof for a sufficient amount of time under amiable conditions, e.g., as recommended in the instructions. A radiolabeled antibody having sufficient purity, specific activity and binding specificity is produced. The radiolabeled antibody may be diluted to an appropriate concentration, e.g., in formulation buffer, and administered directly to the patient without further purification. The chelator-conjugated antibody may be supplied in lyophilized form.

Therapeutic and Prophylactic Methods

The methods of this invention are useful to treat or prevent a disorder, e.g., an insulin-related disorder (e.g., diabetes, e.g., Type 1 or Type 2 diabetes), by administering to a subject a PSMA-binding agent, e.g., an anti-PSMA antibody or antigen-binding fragment thereof, in an amount effective to treat or prevent such disorder.

In some embodiments, the method of the invention can be used, for example, to ablate or kill an aberrant cell, e.g., an aberrant PSMA-expressing cell, or modulate, e.g., reduce activity of PSMA in a PSMA-associated pathway. Thus in some embodiments, the methods of the invention can be used to treat or prevent a disorder, e.g., an insulin-related disorder (e.g., diabetes, e.g., Type 2 diabetes) associated with or secondary to a primary disorder characterized by unwanted cell proliferation, e.g., prostate or non-prostate cancer, or a non-malignant, hyperproliferative disorder, by administering to a subject a PSMA-binding agent, e.g., an anti-PSMA antibody or antigen-binding fragment thereof, in an amount effective to treat or prevent such disorder. Thus in some embodiments, the methods include selecting a population of patients having an insulin-related disorder secondary to a primary disorder characterized by unwanted cell proliferation.

The insulin-related disorder may involve the aberrant activity of a cell or a group of cells or layers in the pancreas (e.g., the islets), insulin resistance, B-cell dysfunction, and/or increase glucose output.

The methods of the invention may be practiced on any subject, e.g., a mammal, a higher primate preferably on humans. As used herein, the term “subject” is intended to include human and non-human animals. Preferred human animals include a human patient having an insulin-related disorder as described herein. The term “non-human animals” includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc.

Examples of insulin-related disorders that can be treated or prevented using the methods of the invention include obesity, hyperglycemia, hypoglycemia, hyperinsulinemia, insulin-resistance, IGT, IFG and diabetes mellitus (e.g., Type 1 or Type 2 diabetes), as well as other disorders involving insulin-stimulated glucose transport. Preferably, the disorder is diabetes mellitus, e.g., Type 2 diabetes.

Most preferably, the disorder is diabetes. The term “diabetes” or “diabetes mellitus” is intended to have its medical meaning, namely, a metabolic disorder of multiple etiology characterized by chronic hyperglycemia with disturbances of carbohydrate, fat and protein metabolism resulting from defects in insulin secretion, insulin action, or both. Symptoms of Type 1 diabetes include polyuria, polydipsia, blurring of vision and unexplained weight loss. Symptoms of Type 2 diabetes include hyperglycemia, hyperinsulinemia and obesity. A diagnosis of diabetes is often made when any three of these tests is positive, followed by a second positive test on a different day:

Fasting plasma glucose of greater than or equal to 126 mg/dl with symptoms of diabetes.

Casual plasma glucose (taken at any time of the day) of greater than or equal to 200 mg/dl with the symptoms of diabetes.

Oral glucose tolerance test (OGTT) value of greater than or equal to 200 mg/dl measured at a two-hour interval. The OGTT is given over a three-hour time span.

Diabetes is also diagnosed by a drop in glycated hemoglobin levels of about 1%.

The effects of diabetes mellitus include long-term damage, dysfunction and failure of various organs. In its most severe forms, ketoacidosis or a non-ketotic hyperosmolar state may develop and lead to stupor, coma and, in absence of effective treatment, death. Often symptoms are not severe, or may be absent, and consequently hyperglycemia sufficient to cause pathological and functional changes may be present for a long time before the diagnosis is made. The long-term effects of diabetes mellitus include progressive development of the specific complications of retinopathy with potential blindness, nephropathy that may lead to renal failure, and/or neuropathy with risk of foot ulcers, amputation, Charcot joints, and features of autonomic dysfunction, including sexual dysfunction. People with diabetes are at increased risk of cardiovascular, peripheral vascular and cerebrovascular disease.

Several pathogenetic processes are involved in the development of diabetes. The abnormalities of carbohydrate, fat and protein metabolism are due to deficient action of insulin on target tissues resulting from insensitivity or lack of insulin. Pathological indications of Type I diabetes include a reduction in number and/or size of pancreatic islet β-cells and high presence of lymphatic infiltrates in an around the islets. These lead to consequent insulin deficiency and glucose intolerance. The pathology of Type 2 diabetes includes fibrotic and/or amylin deposits in the islets of the pancreas, and/or a reduction in the size or number of pancreatic islet β-cells.

In other embodiments, the insulin-related disorder is IGT, which may be diagnosed by the fasting plasma glucose test (FPG) or the oral glucose tolerance test (OGTT). IGT is a condition in which the blood sugar level is elevated (between 140 and 199 milligrams per deciliter or mg/dL after a 2-hour oral glucose tolerance test), but is not high enough to be classified as diabetes.

In other embodiments, the insulin-related disorder is impaired fasting glycemia (or impaired fasting glucose) (IFG). Abnormal blood glucose levels as measured by fasting plasma glucose test (FPG) indicates IFG. IFG is a condition in which the fasting blood sugar level is elevated (between 110 and 125 mg/dL after an overnight fast) but is not high enough to be classified as diabetes.

Both IFG and IGT are considered risk factors for developing diabetes.

In other embodiments, the insulin-related disorder is gestational diabetes, or GDM. GDM maybe diagnosed on the basis of plasma glucose values of ≧105 mg/dl, >190 mg/dl, ≧165 mg/dl, ≧145 mg/dl for the fasting, 1 hour, 2-hour, and 3-hour samples, respectively, were considered abnormal. Two readings should exceed or equal these values to give a diagnosis of GDM. The offspring of mothers experiencing GDM are at a higher risk of intrauterine death or neonatal death.

In other embodiments, the methods of the invention can be used to treat insulin-related disorders, as described herein, that are secondary to, associated with, aggravated by or exacerbated by, a primary malignant or non-malignant, hyperproliferative disorders, e.g., cancers, or other disorders associated with expression of PSMA in the neovasculature. The invention provides a method of treating, e.g., ablating or killing, a cell, e.g., a prostatic cell (e.g., a cancerous or non-cancerous prostatic cell, e.g., a normal, benign or hyperplastic prostatic epithelial cell), or a malignant, non-prostatic cell, e.g., a cell found in a non-prostatic solid tumor, a soft tissue tumor, or a metastatic lesion (e.g., a cell found in renal, urothelial (e.g., bladder), testicular, colon, rectal, lung (e.g., non-small cell lung carcinoma), breast, liver, neural (e.g., neuroendocrine), glial (e.g., glioblastoma), pancreatic (e.g., pancreatic duct) cancer and/or metastasis, melanoma (e.g., malignant melanoma), or soft tissue sarcoma). Treatment of the primary disorder by the methods of the invention can result in treatment of the secondary insulin-related disorder.

In one aspect, the methods of the invention can be used to treat insulin-related disorders, as described herein, that are secondary to, associated with, aggravated by or exacerbated by, a primary prostatic disorder. Examples of prostatic disorders include, but are not limited to, genitourinary inflammation (e.g., inflammation of smooth muscle cells) as in prostatitis; benign enlargement, for example, nodular hyperplasia (benign prostatic hypertrophy or hyperplasia); and cancer, e.g., adenocarcinoma or carcinoma, of the prostate and/or testicular tumors.

As used herein, the term “cancer” is meant to include all types of cancerous growths or oncogenic processes, metastatic tissues or malignantly transformed cells, tissues, or organs, irrespective of histopathologic type or stage of invasiveness.

Examples of non-prostatic cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx. Adenocarcinomas include malignancies such as most colon cancers, rectal cancer, renal-cell carcinoma, liver cancer, non-small cell carcinoma of the lung, cancer of the small intestine and cancer of the esophagus. Metastatic lesions of the aforementioned cancers can also be treated or prevented using the methods and compositions of the invention.

The insulin-related disorder as described herein can be secondary to, associated with, aggravated by, or exacerbated by malignancies of the various organ systems, such as those affecting lung, breast, lymphoid, gastrointestinal (e.g., colon), bladder, genitourinary tract (e.g., prostate), pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods and compositions of the invention can be used to treat or prevent an insulin-related disorder in a subject having a primary condition such as a skin disorder. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, e.g., a dermal endothelial, fibroblast, immune cell (e.g., mast cell or macrophage) found in a dermal layer, e.g., the papillary layer or the reticular layer.

Examples of skin disorders include psoriasis, psoriatic arthritis, dermatitis (eczema), e.g., exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitites such as seborrheic dermatitis and solar dermatitis; keratoses such as seborrheic keratosis, senile keratosis, actinic keratosis. photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. Preferably, the co-existing, aggravating or precipitating disorder is dermatitis, e.g., atopic dermatitis or allergic dermatitis, or psoriasis.

In some embodiments, the skin disorder is psoriasis. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, psoriasis vulgaris, eruptive (gluttate) psoriasis, psoriatic erythroderma, generalized pustular psoriasis (Von Zumbusch), annular pustular psoriasis, and localized pustular psoriasis.

Methods of the invention include the steps of contacting the cell, or a nearby cell, e.g., a vascular endothelial cell proximate to the cell, with a modified anti-PSMA antibody, e.g., a modified anti-PSMA antibody as described herein, in an amount sufficient to treat, e.g., ablate or kill, the cell. The anti-PSMA antibodies, e.g., the modified anti-PSMA antibodies, or antigen-binding fragments thereof, can be used to kill or ablate cancerous cells and normal, benign hyperplastic, and cancerous prostate epithelial cells in vivo. For example, the anti-PSMA antibodies can be used to treat or prevent an insulin-related disorder, e.g., as described herein. The antibodies, e.g., the modified antibodies, (or fragments thereof) can be used by themselves or conjugated to a second agent, e.g., a cytotoxic drug, radioisotope, or a protein, e.g., a protein toxin or a viral protein. This method includes: administering the modified antibody, alone or conjugated to a cytotoxic drug, to a subject requiring such treatment.

The antibodies of the present invention may be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g., via a viral coat protein), or mixtures thereof. The therapeutic agent can be an intracellularly active drug or other agent, such as short-range radiation emitters, including, for example, short-range, high-energy α-emitters, as described herein. In some preferred embodiments, the anti-PSMA antibody, or antigen binding fragment thereof, can be coupled to a molecule of plant or bacterial origin (or derivative thereof), e.g., a maytansinoid (e.g., maytansinol or the DM1 maytansinoid). DM1 is a sulfhydryl-containing derivative of maytansine that can be linked to antibodies via a disulfide linker that releases DM1 when inside target cells. The disulfide linkers display greater stability in storage and in serum than other linkers. Maytansine is a cytotoxic agent that effects cell killing by preventing the formation of microtubules and depolymerization of extant microtubules. It is 100- to 1000-fold more cytotoxic than anticancer agents such as doxorubicin, methotrexate, and vinca alkyloid, which are currently in clinical use. Alternatively, the anti-PSMA antibody, or antigen binding fragment thereof, can be coupled to a taxane, a calicheamicin, a proteosome inhibitor, or a topoisomerase inhibitor. [(1R)-3-methyl-1-[[(2S)-1-oxo-3-phenyl-2-[(3-mercaptoacetyl)amino]propyl]amino]butyl]Boronic acid is a suitable proteosome inhibitor. N,N′-bis[2-(9-methylphenazine-1-carboxamido)ethyl]-1,2-ethanediamine is a suitable topoisomerase inhibitor.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. In a preferred embodiment, the anti-PSMA antibody is conjugated to maytansinoids, e.g., maytansinol (see U.S. Pat. No. 5,208,020), CC-1065 (see U.S. Pat. Nos. 5,475,092, 5,585,499, 5,846,545). Procedures for preparing enzymatically active polypeptides of the immunotoxins are described in WO84/03508 and WO85/03508, which are hereby incorporated by reference, and in the appended Examples below. Examples of cytotoxic moieties that can be conjugated to the antibodies include adriamycin, chlorambucil, daunomycin, methotrexate, neocarzinostatin, and platinum.

In some embodiments, the method can include administering a first antibody, e.g., a modified antibody, conjugated with a prodrug which is activated only when in close proximity with a prodrug activator. The prodrug activator is conjugated with a second antibody, e.g., a second modified antibody according to the present invention, preferably one that binds to a non-competing site on the prostate specific membrane antigen molecule. Whether two modified antibodies bind to competing or non-competing binding sites can be determined by conventional competitive binding assays. For example, monoclonal antibodies J591, J533, and E99 bind to competing binding sites on the prostate specific membrane antigen molecule. Monoclonal antibody J415, on the other hand, binds to a binding site that is non-competing with the site to which J591, J533, and E99 bind. Thus, for example, the first modified antibody can be one of J591, J533, and E99, and the second modified antibody can be J415. Alternatively, the first modified antibody can be J415, and the second modified antibody can be one of J591, J533, and E99. Drug-prodrug pairs suitable for use in the practice of the present invention are described in Blakely et al., “ZD2767, an Improved System for Antibody-directed Enzyme Prodrug Therapy That Results in Tumor Regressions in Colorectal Tumor Xenografts,” (1996) Cancer Research, 56:3287-3292, which is hereby incorporated by reference.

Alternatively, the antibody, e.g., the modified antibody, can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985), which is hereby incorporated by reference. Other suitable radioisotopes include β-emitters, such as ²¹²Bi, ²¹³Bi, and ²¹¹At, and β-emitters, such as ¹⁸⁶Re and ⁹⁰Y. Radiotherapy is expected to be particularly effective, because prostate epithelial cells and vascular endothelial cells within cancers are relatively radiosensitive. Moreover, Lu¹¹⁷ may also be used as both an imaging and cytotoxic agent.

The antibodies of the invention can also be conjugated or fused to viral surface proteins present on viral particles. For example, a single-chain anti-PSMA antibody of the present invention could be fused (e.g., to form a fusion protein) to a viral surface protein. Alternatively, a whole anti-PSMA antibody of the present invention, or a fragment thereof, could be chemically conjugated (e.g., via a chemical linker) to a viral surface protein. Preferably, the virus is one that fuses with endocytic membranes, e.g., an influenza virus, such that the virus is internalized along with the anti-PSMA antibody and thereby infects PSMA-expressing cells. The virus can be genetically engineered as a cellular toxin. For example, the virus could express or induce the expression of genes that are toxic to cells, e.g., cell death promoting genes. Preferably, such viruses would be incapable of viral replication.

The antibodies, e.g., the modified antibodies of the invention, can be used directly in vivo to eliminate antigen-expressing cells via natural complement or antibody-dependent cellular cytotoxicity (ADCC). Modified antibody molecules of the invention, which have complement binding sites, such as portions from IgG1, -2, or -3 or IgM which bind complement can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with modified antibodies or fragments thereof of the invention can be improved by binding of complement proteins. In another embodiment target cells coated with the modified antibodies or fragments thereof can also be lysed by complement.

Combination Therapy

The PSMA binding agents, e.g., anti-PSMA antibodies or antigen-binding fragments thereof, may be used in combination with other therapies. For example, the combination therapy can include a composition of the present invention coformulated with, and/or coadministered with, one or more additional therapeutic agents, e.g., one or more anti-cancer agents, cytotoxic or cytostatic agents and/or immunosuppressants. In other embodiments, the subject is administered a PSMA binding agent separately from the administration of the other therapy. The terms “cytotoxic agent” and “cytostatic agent” are used interchangeably herein and refer to agents that have the property of inhibiting the growth or proliferation (e.g., a cytostatic agent), or inducing the killing, of hyperproliferative cells, e.g., an aberrant T cell.

For example, the PSMA binding agents may be coformulated with, and/or coadministered with, one or more additional antibodies that bind other targets (e.g., antibodies that bind other cytokines or that bind cell surface molecules), including naked antibodies, immunotoxins, and radioconjugates, one or more cytokines, or immunosuppressants, e.g., cyclosporin A or FK506.

For insulin-related disorders, combination of PSMA binding agent with current therapeutic modalities for insulin-related disorders are preferred. Examples of such modalities include administration of insulin, sulfonylureas (e.g., meglitinides and nateglinides), biguanides, thiazolidinediones, and alpha-glucosidase inhibitors. Such combination therapy may in some cases advantageously utilize lower dosages of the therapeutic or prophylactic agents. Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. In some embodiments, the combination therapy results in a reduced dose of the therapeutic modality for the insulin-related disorder, e.g., the dose is reduced by at least 5%, 10%, 20%, 30%, 40%, 50% or more as compared to the dose in the absence of administration of the PSMA binding agent. In other embodiments, the combination therapy results in extended periods between administration of the therapeutic modality, e.g., the period between administration of the modality is at least 1, day, 2 days, 3, days, 4 days, 5 days, a week, two weeks, three weeks, a month, 2 months, three months or more as compared to the time between administrations in the absence of administration of the PSMA binding agent.

Combinations of PSMA binding agents, e.g., anti-PSMA antibodies, with one or more additional antibodies or ligands that bind other targets (e.g., antibodies that bind other cytokines or that bind cell surface molecules), including naked antibodies, fusion proteins, immunotoxins, and radioconjugates, can be used. Example of antibodies that can be used in combination with PSMA binding agents include antibodies against IL-8 (ABX-IL8 (Abgenix)); complement C5 protein (5G1.1 (Alexion)); CD2 (MEDI-507/BTI-322 (MedImmune/BioTransplant)); E selectin (CDP 850 (Celltech)); TNF alpha (Remicaide (Centocor)); CD4 (HuMax-CD4 (Genmab)); IL15 (HuMax-IL15 (Genmab/Immunex)); ICAM-3 (ICM3 (Icos); CD64 (MDX-44 (Medarex)); IL2-receptor (Zenepax (PDL)); CD3 (Nuvion (PDL)); and CD11a (Xanelim (Genentech/Xoma)). In addition to the above, immunoglobulin fusion proteins that bind to other targets can be used. For example, immunoglobulin fusions that bind to CD2, e.g., LFA-3-Ig, can be used.

Administered “in combination”, as used herein, means that two (or more) different treatments are delivered to the subject during the course of the subject's affliction with the disorder or disorders, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. E.g., the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In a preferred embodiment a delivery of the first treatment and a delivery of the second treatment occur within 1, 2, 5, 10, 15, or 30 days of one another.

The binding agents as described herein can be used as an adjunct to conventional treatments of insulin-related conditions, such as diabetes. For example, binding agents can be introduced prior to, concurrently with, or after pharmaceutical therapy for diabetes, e.g., the administration of insulin, sulfonylureas (e.g., meglitinides and nateglinides), biguanides, thiazolidinediones, and/or alpha-glucosidase inhibitors.

In other exemplary embodiments, a PSMA-binding agent can be administered over an extended period of time (e.g., a therapeutic treatment period of twelve weeks). During periods of remission or less active disease, the PSMA-binding agent can be administered alone or in combination with a second agent (e.g., insulin, sulfonylureas (e.g., meglitinides and nateglinides), biguanides, thiazolidinediones, and alpha-glucosidase inhibitors). During periods of active disease, a rapidly acting, but toxic auxiliary agent, such as methotrexate and/or cyclosporin, can be administered for a short treatment period, in addition to or in combination with the binding agent and the second agent.

In a preferred embodiment, the PSMA-binding agent (e.g., anti-PSMA antibody) or a pharmaceutical composition containing the same is administered systemically (e.g., intravenously, intramuscularly, subcutaneously, intraarticularly, intrathecally, periostally, intratumorally, intralesionally, perilesionally by infusion (e.g., using an infusion device), orally, topically or by inhalation). Preferably, the PSMA-binding agent is administered intramuscularly or intravenously. In other embodiments, the PSMA-binding agent is administered locally to an affected area.

Pharmacogenomics

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. See, for example, Eichelbaum, M. et al. (1996) Clin. Exp. Pharmacol. Physiol. 23:983-985 and Linder, M. W. et al. (1997) Clin. Chem. 43:254-266. Differences in metabolism of therapeutics can lead to severe toxicity or therapeutic failure by altering the relation between dose and blood concentration of the pharmacologically active drug. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment according to that individual's drug response genotype.

Information generated from pharmacogenomic research can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when administering a therapeutic composition, e.g., a composition consisting of one or more PSMA binding agents, or derivatized form(s) thereof, to a patient, as a means of treating a disorder, e.g., an insulin-related disorder as described herein.

In one embodiment, a physician or clinician may consider applying knowledge obtained in relevant pharmacogenomics studies when determining whether to administer a pharmaceutical composition, e.g., a composition consisting of one or more PSMA-binding agents, or derivatized form(s) thereof, and optionally a second agent, to a subject. In another embodiment, a physician or clinician may consider applying such knowledge when determining the dosage, e.g., amount per treatment or frequency of treatments, of a pharmaceutical composition, e.g., a pharmaceutical composition as described herein, administered to a patient.

In yet another embodiment, a physician or clinician may determine the genotypes, at one or more genetic loci, of a group of subjects participating in a clinical trial, wherein the subjects display a disorder, e.g., an insulin-related disorder as described herein, and the clinical trial is designed to test the efficacy of a pharmaceutical composition, e.g., a composition consisting of one or more PSMA-binding agents, and optionally a second agent, and wherein the physician or clinician attempts to correlate the genotypes of the subjects with their response to the pharmaceutical composition.

Diagnostic Uses

In one aspect, the present invention provides a diagnostic method for detecting the presence of PSMA, in vitro (e.g., a biological sample, such as plasma, tissue, biopsy, e.g., a pancreatic tissue) or in vivo (e.g., in vivo imaging in a subject). The method includes: (i) contacting the sample with a PSMA binding agent, or administering to the subject, the PSMA binding agent; (ii) contacting a control sample (e.g., a control biological sample, such as plasma, tissue, biopsy) or a control subject)); and (iii) detecting formation of a complex between the PSMA binding agent, and the sample or subject, or the control sample or subject, wherein a statistically significant change in the formation of the complex in the sample or subject relative to the control sample or subject is indicative of the presence of the antigen in the sample.

Preferably, the PSMA binding agent is directly or indirectly labeled with a detectable substance to facilitate detection of the bound or unbound antibody. Suitable detectable substances include various enzymes, prosthetic groups, fluorescent materials, luminescent materials and radioactive materials, as described above. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Complex formation between the PSMA binding agent and PSMA can be detected by measuring or visualizing either the antibody (or antibody fragment) bound to the PSMA antigen or unbound antibody (or antibody fragment). Conventional detection assays can be used, e.g., an enzyme linked immunosorbent assays (ELISA), an radioimmunoassay (RIA) or tissue immunohistochemistry.

Alternative to labeling the PSMA binding agent, the presence of PSMA can be assayed in a sample by a competition immunoassay utilizing standards labeled with a detectable substance and an unlabeled anti-PSMA antibody. In this assay, the biological sample, the labeled standards and the PSMA binding agent are combined and the amount of labeled standard bound to the unlabeled antibody is determined. The amount of PSMA in the sample is inversely proportional to the amount of labeled standard bound to the PSMA binding agent.

In still another embodiment, the invention provides a method for detecting the presence of a PSMA-expressing cell in vivo. The method comprises (i) administering to a subject (e.g., a patient having an insulin-related disorder, e.g., diabetes mellitus) a PSMA binding agent, conjugated to a detectable marker; (ii) exposing the subject to a means for detecting said detectable marker to the PSMA-expressing cells. Protocols for in vivo diagnostic assays are provided in PCT/US88/01941, EP 0 365 997 and U.S. Pat. No. 4,954,617.

In another aspect, the invention features methods for detecting the presence of a PSMA nucleic acid, e.g., mRNA or cDNA, or PSMA protein, in a sample, in vitro (e.g., a biological sample, such as plasma, tissue biopsy, e.g., a psoriatic lesion). The subject method can be used to evaluate, e.g., diagnose or stage a disorder described herein, e.g., an insulin-related disorder, e.g., diabetes. The method includes: (i) contacting the sample (and optionally, a reference, e.g., a control sample) with an agent specific for a PSMA nucleic acid, e.g., a probe or a primer, or a PSMA binding agent, under conditions that allow interaction of the agent and the PSMA nucleic acid, e.g., mRNA or cDNA, or protein to occur; and (ii) detecting formation of a complex between the agent, and the sample (and optionally, a reference, e.g., a control sample). Formation of the complex is indicative of the presence of PSMA nucleic acid or protein, and can indicate the suitability or need for a treatment described herein. For example, a statistically significant change in the formation of the complex in the sample relative to the control sample is indicative of the presence of PSMA in the sample. In one embodiment, the PSMA-binding agent is an anti-PSMA antibody, or antigen-binding fragment thereof, e.g., an anti-PSMA antibody, or antigen-binding fragment thereof as described herein. In other embodiments, the agent is a nucleic acid that specifically hybridizes to the PSMA nucleic acid.

In yet another aspect, the invention provides a method for detecting the presence of PSMA, in vivo (e.g., in vivo imaging in a subject). The subject method can be used to evaluate, e.g., diagnose or stage a disorder described herein, e.g., an insulin-related disorder, e.g., diabetes in a subject, e.g., a mammal, e.g., a primate, e.g., a human. The method includes: (i) administering to a subject (and optionally, a reference, e.g., a control subject) a PSMA binding agent, under conditions that allow interaction of the binding agent and the PSMA protein to occur; and (ii) detecting formation of a complex between the PSMA binding agent and PSMA. A statistically significant change in the formation of the complex in the subject relative to the reference, e.g., the control subject or subject's baseline, is indicative of the presence of PSMA.

In other embodiments, a method of diagnosing or staging, a disorder described herein, e.g., an insulin-related disorder, e.g., diabetes, is provided. The method includes: (i) identifying a subject having, or at risk of having, an insulin-related disorder, e.g., diabetes; (ii) obtaining a sample of a tissue or cell affected with the disorder; (iii) contacting said sample or a control sample with a labeled agent specific for a PSMA nucleic acid, e.g., a probe or a primer, or a labeled PSMA binding agent, under conditions that allow interaction of the binding agent and the PSMA nucleic acid, e.g., cDNA, mRNA, or PSMA protein to occur, and (iv) detecting formation of a complex. A statistically significant increase in the formation of the complex between the labeled agent with respect to a control sample is indicative of the insulin-related disorder or the stage of the disorder.

Preferably, the agent, e.g., the PSMA binding agent, e.g., the anti-PSMA antibody or fragment thereof, is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, and radioactive materials. In some embodiments, the modified anti-PSMA antibody or fragment thereof is coupled to a radioactive ion, e.g., indium (¹¹¹In), iodine (¹³¹I or ¹²⁵I), yttrium (⁹⁰Y), lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (⁹⁹mTc), praseodymium, or phosphorous (³²P).

The following invention is further illustrated by the following examples, which should not be construed as further limiting. The contents of all references, pending patent applications and published patents, cited throughout this application are hereby expressly incorporated by reference.

### EXAMPLES

### Example 1

### Treatment of Diabetes Mellitus Type 2 Using Deimmunized J591 Antibody

A male patient was diagnosed with metastatic prostate cancer and diabetes in 1994. Initially, the patient's diabetes was treated with Glipizide and Glucophage until he developed an allergic reaction to these drugs. Subsequently, he was begun on injectable insulin. The patients required insulin injections twice a day, 8 u in the morning and 6 u in the evening.

In 2002, the patient was treated with a single dose of J591-¹⁷⁷Lutetium for his prostate cancer. Prior to receiving this dose, the patient was taking the following medications: MSO₄ at 30 mg po q 4, Lasix at 40 mg qd, Senokot at 2 tabs bid, Colace at 100 mg bid, Lactulose at 1 tsp prn, Insulin at 8 u in the morning and 6 u in the evening (Humalog 75/25), Plavix at 75 mg qd, Erythromycin at 333 mg pre meals, Kayexylate at 60 ml qd, and Epogen at q week. The patient was weaned off of all of is medications except Celexa, Lupron and potentially Epogen. Prior to treatment, the patient was also on the Duragesic patch at 50 mcg, after treatment with J591-¹⁷⁷Lu, the dose was weaned to 25 mcg, and eventually was stopped.

After treatment with J591-¹⁷⁷Lu, the patient reported a significant relief of pain. Apparently, he had a palpable sternal mass. The patient reported sternal pain improved and the mass has decreased in size. The other change—is that he had no insulin requirements for over two weeks. The patients BGMs were tested three times a day over this period and were reported to range from 70 s-160 s and no insulin injections were required during that period. This is in comparison to his previous requirement of two injections a day.

### Example 2

### Chelation of Anti-PSMA Antibodies to ¹¹¹Indium, ⁹⁰Yttrium, and ¹⁷⁷Lutetium

The anti-PSMA monoclonal antibody of the present invention can be radiolabeled with ¹¹¹Indium, ⁹⁰Yttrium and ¹⁷⁷Lutetium coupled with 1,4,7,10-tetraazacyclododecane-N,N′,N″,N′″-tetraacetic acid (DOTA).

For example, as detailed below, the modified anti-PSMA monoclonal antibodies can be radiolabeled with ¹¹¹Indium, ⁹⁰Yttrium, or ¹⁷⁷Lutetium by directly coupling one of the four carboxylic acid groups of the chelator 1,4,7,10-tetraazacyclododecane-N,N′,N″,N′″-tetraacetic acid (DOTA) to primary amines present on the surface of the antibodies. The DOTA conjugated antibody is then purified, sterile filtered, and vialed. Prior to use, the purified antibodies can be mixed with the desired radiolabel which binds to DOTA.

Chelation Process

Monoclonal antibody deJ591 was conjugated with 1,4,7,10-tetraazacyclododecane-N,N″,N″,N′″-tetraacetic acid (DOTA) and subsequently radiolabeled with ¹¹¹In, ⁹⁰Y and ¹⁷⁷Lu. Radiolabeling and quality control tests were performed on three separate vials of clinical grade mAb deJ591.

All reagents used in the conjugation and purification of deJ591 were made from pyrogen-free water. In the specific case of NH₄OAC buffer and sodium phosphate buffer, the solutions were purified with Chelex 100 (Bio-Rad, CA) to remove any metal ions.

Conjugation of Antibody with 1,4,7,10-Tetraazacyclododecane-N,N′,N″,N′″-tetraacetic Acid (DOTA)

The monoclonal antibody deJ591 was modified with 1,4,7,10-tetraazacyclododecane-N,N′,N″,N′″-tetraacetic acid (DOTA) as follows. Briefly, 25 mg of deJ591 was concentrated in a 30 kDa microsep centrifugal concentrator (Pall Filtron, MA) and washed with 5×4 mL of 1% DTPA (pH 5.0), over a period of 24 hours. The antibody buffer was then changed to 0.1 M phosphate (pH 7.0) using the same centrifugal technique. An active ester of DOTA was created by dissolving 146 mg DOTA (0.361 mmoles) and 36 mg N-hydroxysuccinimide (0.313 mmoles) in 2 ml of water and adjusting the pH to 7.3 with NaOH, prior to the addition of 10 mg of 1-ethyl-3-(3-dimethylaminopropyl)carbodiimide (see below). This reaction mixture was cooled on ice for 1 hour before being added to the deJ591 solution. The resultant DOTA-deJ591 was separated from the excess DOTA and other reactants by repeated washing with 0.3 M NH₄OAc (20×4 mL) and centrifugal concentration. The purified conjugate was then sterilized by filtration through a 0.22 μm filter and stored in a sterile polypropylene vial at 4° C.

The concentration of the DOTA-deJ591 conjugate was assayed by determining the UV absorption at 280 nm and two 50 μL aliquots mixed with either 20 or 30 μL of a 1.30 mM solution of InCl₃ (0.01 M HCl) spiked with a tracer amount of ¹¹¹In. The mixture is incubated at 37° C. for 16 hours and then analyzed by ITLC, using silica gel impregnated glass fiber 10 cm strip (ITLC-SG, Gelman, prod. # 61885) and an eluant of 1% DTPA (pH 6.0). The antibody bound activity remains at the origin and free ¹¹¹In moves with the solvent front as an [In-DTPA] complex. The relative amounts of ¹¹¹In and ¹¹¹In-DOTA-J591 is determined by cutting the ITLC strip at a R_{f} of 0.5 and counting the two halves with a Na(Tl)I detector. The number of binding sites is calculated by considering the molar reaction ratio between ¹¹¹In and DOTA-deJ591 and the observed ratio of ¹¹¹In and ¹¹¹In-DOTA-J591 detected. Typically, 5.1 molecules of DOTA are conjugated to deJ591. Table 9 shows the results from two conjugations of deJ591.

TABLE 9
Calculation of the Mean Number of DOTA
Molecules Conjugated to deJ591

| | Known | Observed | Mean number of |
|---|---|---|---|
| | ¹¹¹In/DOTA-J591 | ¹¹¹In/DOTA-J591 | DOTA mols per |
| Test number | Reaction ratio | TLC ratio | mAb |
| A | 11.76 | 1.338 | 5.03 |
| B | 17.64 | 2.469 | 5.09 |

Radiolabeling

The following radiolabeling procedure is described for ¹¹¹In, but may be used with other radiolabels such as ⁹⁰Y or ¹⁷⁷Lu. Radiolabeling was achieved by adding the ¹¹¹In (in dilute HCl) to the ammonium acetate buffered DOTA-deJ591. To avoid the effects of autoradiolysis on the antibody, the reaction time was minimized and the reaction mixture purified with a size exclusion column prior to administration. Briefly, a mixture composed of 20 μL of ¹¹¹InCl₃ (8 mCi, 0.01 M HCl, 400 μL DOTA-deJ591 (4 mg/ml, 0.3 M NH₄OAc, pH 7) was allowed to react at 37° C. for 20 minutes. The reaction mixture was then separated on a 16 mL Biogel-P6DG column (Bio-Rad, CA) equilibrated with 4×10 mL of sterile 1% HSA in PBS (HSA meets specification for US licensed albumin; manufactured by Central Laboratory Blood Transfusion Service Swiss Red Cross, Bern, Switzerland, License No. 647). Once the reaction mixture was loaded onto the column, it was washed with a further 2 mL of 1% HSA PBS, before the main ¹¹¹In-DOTA-deJ591 fraction was eluted with 5 mL of 1% HSA PBS. The purified ¹¹¹In-DOTA-deJ591 was then sterile filtered into a sterile evacuated vial. Using this method, specific activity of 7.6 mCi ¹¹¹In/mg DOTA-deJ591 was achieved.

Radiochemical Purity

The amount of free ¹¹¹In in radiolabeled DOTA-deJ591 preparations was evaluated using the instant thin layer chromatography method with a silica gel impregnated glass fiber support and a mobile phase of 1% DTPA (pH 5.5). Briefly, a portion of the radiolabeled DOTA-deJ591 was spotted on a 10 cm ITLC-SG strip (Gelman, prod. # 61885) and developed in 1% DTPA (pH 5.5). Once the solvent front had reached the end of the strip, it was removed from the solvent and cut at a R_{f} of 0.5. The two portions were assayed for radioactivity and the radiochemical purity determined using the following equation:

Radiochemical purity=(Activity in between R_{f} 0 and 0.5)/(Total activity in strip)

Immunoreactivity

The immunoreactivity of the ¹¹¹In-DOTA-deJ591 preparations was assessed according to the method of Lindmo (Lindmo T. et al. (1994) J. Immunol. Methods, 72:77-89, 1994) that extrapolates the binding of the radiolabeled antibody at an infinite excess antigen. Briefly, five test solutions were prepared (in duplicate) containing 10,000 cpm of ¹¹¹In-DOTA-deJ591 and various amounts of LNCaP cells, in a total test volume of 250 μL of 0.2% BSA 10 mM HEPES. The solutions were incubated at 4° C. for 60 minutes prior to being isolated (by centrifugation) and washed with ice cold PBS. The membranes were then counted in a gamma counter with standards representing the total radioactivity added. The data was plotted using the Lindmo method as the reciprocal of the substrate concentration x-axis) against the reciprocal of the fraction bound (y-axis). The data was then fitted according to a least squares linear regression method (Sigma Plot) and the y intercept taken as the reciprocal of the immunoreactivity. A similar method using membranes derived from LNCaP cells, and subsequent centrifugation isolation of the membranes, gave similar results. The results gave an average immunoreactivity of 72% (see Table 9).

Immunohistochemistry

Immunohistochemistry was performed on the DOTA conjugated, partially purified, bulk intermediate deJ591. The results showed that the preparation was specific to prostate tissue and the reactivity was equivalent to the naked deJ591 antibody.

Sterility

The sterility of ¹¹¹In-DOTA-deJ591 preparations was determined using thioglycollate medium according to the USP 24/NF 19 method. Briefly, quadruplicate 0.1 mL samples of the ¹¹¹In-DOTA-deJ591 preparations were transferred to 15 mL of fluid thioglycollate medium and the mixture incubated at 35° C. for 14 days. The media were visually inspected on the 4th, 7th and 14 days of any signs of growth. All three preparations showed no growth (See Table 9).

Endotoxin

The endotoxin of ¹¹¹In-DOTA-deJ591 preparations was determined using the Limulus amebocyte lysate assay according to the USP 24/NF 19. Briefly, a Limulus amebocyte lysate kit (Bio Whittaker lot # 7L3790, sensitivity 0.125 EU/mL) was reconstituted with 0.25 mL of test sample. The quadruplicate test samples, artificially positive test samples, negative controls and positive controls were incubated at 37° C. for 60 minutes. Positive results were typified by the formation of a viscous gel that was unaffected by 180° inversion. The single preparation gave a value of less than 5 EU/mL. This assay can (and will) be repeated on the patient dose immediately prior to administration.

TABLE 10
Analytical Results of Radiolabeled ¹¹¹In-DOTA-deJ591

| Test | Result |
|---|---|
| Radiolabeling yield | 85% |
| Radiochemical Purity | >99% |
| Immunoreactivity | 72% |
| Endotoxin | <5 Eu/mL |
| Sterility | Sterile |

Lot # of dej591: BIOV983.2-2

Large-Scale Manufacture/Process

The large-scale manufacture of the DOTA conjugated deJ591 antibody is described in the following paragraphs. The major differences from the above methodology were the use of a stirred cell, instead of a microsep centrifugal concentrator to concentrate and diafilter the antibody and the use of a Sephadex G-25 column to remove the unreacted DOTA and other reagents from the DOTA conjugated antibody. These changes were necessitated by the increase in scale. The ratios of the starting materials are given in Table 11 for a nominal 1000 milligram scale. The process may be scaled up using equivalent ratios of starting materials.

TABLE 11
Unit Ratios of Starting Materials

| Starting Material | Unit Ratio | |
|---|---|---|
| deJ591 antibody | | X mgs |
| 1,4,7,10-tetraazacyclododecane-N,N′,N′′,N′′′- | 1.25 | X mgs |
| tetraacetic acid (DOTA) | | |
| N-hydroxysuccinimide (NHS) | 0.275 | X mgs |
| 1-Ethyl-3-(3-dimethylaminopropyl) | 0.3 | X mgs |
| carbodiimide (EDC) | | |

Aseptic practices were observed in order to minimize contamination and environmental monitoring was conducted at periodic intervals during the manufacture. All solutions, buffers and reagents used in the conjugation and purification of DOTA-deJ591 antibody were made with Water For Injection (WFI). Throughout the process, metal free components were used in the manufacture in order to avoid the chelation of any free metal residues by the DOTA moiety. In the specific case of ammonium acetate buffer and sodium phosphate buffer, the solutions were purified with Chelex 100 to remove any metal ions. Sterile, pyrogen free and metal free containers were used to mix reactants. The final bulk sterile filtration was conducted in an area that meets Class 100 specifications.

The deJ591 was prepared by buffer exchanging the antibody into metal free, 0.1 M Sodium Phosphate, pH 7.1, over a Chelex 100 (BioRad or equivalent) column. The antibody was then concentrated to approximately 10 mg/mL using a Stirred Cell Unit (Millipore or equivalent) equipped with a 30 kD cut-off membrane. The concentrated antibody was then sterile-filtered through a 0.22 μm filter.

To conjugate one gram of antibody, the active ester of DOTA was prepared by adding 6.3 mL of 0.49 M DOTA in metal free, Sodium Phosphate Buffer, pH 7.1, to 2.7 mL of 0.87 M N-hydroxysuccinimide in metal free, Sodium Phosphate Buffer, pH 7.1. To this mixture, 0.1 N Sodium Hydroxide was added until the DOTA was completely dissolved (approximately a 1:1 ratio of 0.1 M Sodium Hydroxide to DOTA/NHS solution). The pH was between 6.9 and 7.2. The solution was cooled for not less than 30 minutes at 2-8° C. To the DOTA/NHS solution, 1.5 mL of 1.0 M of EDC in Sodium Phosphate Buffer, pH 7.1, was added and allowed to cool at 2-8° C. for not less than 1 hour.

The active DOTA ester was added to 1 gram of antibody and incubated overnight (12-14 hrs) at 2-8° C. The DOTA conjugated antibody was purified over a Sephadex G-25 column (Pharmacia or equivalent) in metal free, 0.3 M Ammonium Acetate Buffer, pH 7.2. The eluate fraction containing the DOTA conjugated antibody was concentrated using a Stirred Cell equipped with a 30 kD cut-off membrane to approximately 10 mg/mL. The DOTA conjugated deJ591 Antibody was then diafiltered in 0.3 M Ammonium Acetate, pH 7.2 to remove any excess reagents and diluted to a final concentration of 8.0 mg/mL prior to sterile filtering through a 0.22 μm filter.

DOTA conjugated deJ591 was tested for concentration, immunoreactivity, conjugation, endotoxin, and sterility. The endotoxin limit is based on the low clinical dose of the radiolabeled DOTA conjugated deJ591 antibody required, which ranges from 1 to 5 mg. Bioburden testing was performed on the bulk purified DOTA conjugated antibody instead of sterility because of the small batch sizes. Sterility (21 CFR 610) will be performed on the final vialed drug product. The target for immunoreactivity and number of DOTA moles per antibody was based on previous clinical experience. DOTA conjugated antibody with immunoreactivity values of as low as 72% have been successfully used in the clinic. The number of DOTA moles per antibody is based on the results from previous clinical lots.

Protein Concentration

A sample of DOTA-deJ591 was analyzed by optical density in a spectrophotometer at a wavelength of 280 nm. The extinction coefficient used for these calculations was A₂₈₀, E_{1 cm}^{0.1%}=1.4. The test sample was suitably diluted to give an absorbance reading in the working range of the assay (0.2 OD units to 1.2 OD units, linear, CV less than 2%). The acceptable limit for protein concentration is 8.0 mg/mL±0.5 mg/mL.

Endotoxin 1003371 Samples of DOTA-deJ591 were tested for pyrogens using a validated Limulus Amebocyte Lysate test (LAL) Gel Clot Assay (BioWhittaker or equivalent). A 0.06 EU/mL sensitivity Lysate was utilized and samples were diluted either 1:10 or 1:25 in Endotoxin free water for analysis in order to overcome the inhibition level of certain chemicals to the gel clot assay. Duplicate determinations were made for each buffer or intermediate sample during processing and the sample values needed to be equal to or less than the value obtained at the dilution level set for that buffer. A positive and negative control, as well as an inhibition control, was run with every sample. The proposed acceptable limits were not more than 5 EU per mg of DOTA-deJ591.

Bioburden

Aliquots of DOTA-deJ591 were directly inoculated in fluid thioglycollate and soybean-casein broth. The media were examined after fourteen days of incubation. As necessary, both media showed no growth after fourteen days.

Immunoreactivity

The immunoreactivity of the DOTA-deJ591 preparations was assessed according to the method of Lindmo (Lindmo T. et al. (1994) J. Immunol. Methods 72:77-89) which extrapolates the binding of the radiolabeled antibody at an infinite amount of excess antigen. Briefly five test solutions were prepared (in duplicate) containing 10,000 cpm of ¹¹¹Indium labeled-DOTA-deJ591 and various amounts of LNCaP cells or cell membranes, in a total test volume of 250 μL of 0.2% BSA 10 mM HEPES. The solutions were incubated at 4° C. for 60 minutes prior to being isolated (by centrifugation) and washed with ice cold PBS. The membranes were then counted in a gamma counter with standards representing the total radioactivity added. The data was plotted using the Lindmo method as the reciprocal of the substrate concentration x-axis) against the reciprocal of the fraction bound (y-axis). The data was then fitted according to a least squares linear regression method (Sigma Plot) and the y intercept used as the reciprocal of the immunoreactivity. The target for immunoreactivity was not less than 75%.

Number of DOTA Moles per Antibody

The number of DOTA bound per antibody was determined using a saturation binding method with natural occurring isotope of Indium and ¹¹¹Indium. Multiple aliquots (minimum two) of DOTA-deJ591 were mixed with various amounts, ranging from 10 to 30 μL, of a 3.0 mM solution of InCl₃ (0.01 M HCl) spiked with a tracer amount of ¹¹¹In. The mixture was incubated at 37° C. for 16 hours and then analyzed by ITLC, using silica gel impregnated glass fiber 10 cm strip (ITLC-SG, Gelman, or equivalent) and an eluant of 1% DTPA (pH 6.0). The antibody bound activity remains at the origin and free ¹¹¹In moves with the solvent front as an [In-DTPA] complex. The relative amounts of ¹¹¹In and ¹¹¹In-DOTA-J591 was determined by cutting the ITLC strip at a R_{f} of 0.5 and counting the two halves with a Na(Tl)I detector. The number of binding sites was calculated by considering the molar reaction ratio between ¹¹¹In and DOTA-deJ591 and the observed ratio of ¹¹¹In and ¹¹¹In-DOTA-J591 detected. The target number of DOTA molecules per antibody was between 4 and 6.

The analytical results for a sample lot of DOTA conjugated deJ591 antibody are shown below in Table 12.

TABLE 12

| Test | Proposed Acceptable Limits | Results |
|---|---|---|
| Appearance | Clear Colorless Solution | Clear Colorless |
| | | Solution |
| Concentration | 8.0 mg/mL ± 0.5 mg/mL | 8.4 mg/mL |
| Endotoxin | NMT 5 EU per mg | <1.2 EU/mg |
| Bioburden | No growth | No growth |
| Immunoreactivity | For Information Only (Target | 95% |
| | NLT 75%) | |
| Number of DOTA | For Information Only (Target 4-6 | 6 |
| moles per | DOTA per Antibody) | |
| Antibody | | |

The DOTA conjugation numbers for a previous lot of DOTA conjugated antibody (Biov983.2-2) and current Lot 243101 are shown in Table 13. The average number of DOTA moles per antibody for Lot Biov983.2-2 was 5.06 and for Lot 243101 was 5.96. Although the number of moles of DOTA conjugated per antibody was slightly higher for Lot 243101, the immunoreactivity was not affected as shown in Table 14. In fact, the immunoreactivity for Lot 243101 was higher than that for the comparison lot, which is beneficial. It should be noted that other small-scale clinical lots have had immunoreactivity values of greater than 90% (data not shown).

TABLE 13
Comparison of the Mean Number of DOTA Molecules
Conjugated to deJ591 antibody

| | Known | | |
|---|---|---|---|
| | ¹¹¹In/DOTA- | Observed | Mean |
| | deJ591 | ¹¹¹In/DOTA-deJ591 | number of DOTA |
| Lot number | Reaction ratio | TLC ratio | mols per mAb |
| BIOV983.2-2 | | | |
| A | 11.76 | 1.338 | 5.03 |
| B | 17.64 | 2.469 | 5.09 |
| Ave | | | 5.06 |
| Lot 243101 | | | |
| A | 10.98 | 0.8608 | 5.90 |
| B | 16.46 | 1.7301 | 6.03 |
| C | 21.95 | 2.8226 | 5.74 |
| D | 32.93 | 4.3498 | 6.15 |
| Ave | | | 5.96 |

A = 10 μL of In-natural/¹¹¹In solution,
B = 15 μL of In-natural/¹¹¹In solution,
C = 20 μL of In-natural/¹¹¹In solution,
D = 30 μL of In-natural/¹¹¹In solution

TABLE 14
Comparison of Immunoreactivity of DOTA-deJ591

| | | |
|---|---|---|
| Test | Lot BIOV983.2-2 | Lot 243101 |

| | | |
|---|---|---|
| Immunoreactivity | 72% | 95% |

An alternative synthesis is as follows: 956.5 mg of deJ591 was diafiltered six times. The antibody was concentrated in a 30 kDa microsep centrifugal concentrator (Pall Filtron, MA) to approximately 15 mg/mL and diluted 12.5 fold with metal free 0.1 M Sodium phosphate at pH 7.1. This procedure is performed six times. An active ester of DOTA was created by mixing 598 mg DOTA (1.48 mmoles) in 5.95 mL 0.1 M metal free phosphate buffer and 132 mg N-hydroxysuccinimide (1.15 mmoles) in 2.7 ml of 0.1 M metal free phosphate buffer. The pH was adjusted to 6.9-7.2 with NaOH, prior to the addition of 144 mg (0.75 mmoles) of 1-ethyl-3-(3-dimethylaminopropyl)carbodiimide in 1.45 mL 0.1 M metal free phosphate buffer. This reaction mixture was filtered through a 0.2 micron sterile filter and cooled on ice for 1 hour before being added to the deJ591 solution and incubated overnight at 2-8 C for 14-18 hours. The resultant DOTA-deJ591 was separated from the excess DOTA and other reactants by purifying it through a G-25 column equilibrated in 0.3 M metal free ammonium acetate. The purified conjugate was concentrated to 10 mg/mL in a stirred cell unit and washed with 0.3 M ammonium acetate, then sterilized by filtration through a 0.22 um filter and stored in a sterile polypropylene vial at 2-8° C.

### Example 3

### Use of the mAbs for Targeted Delivery of Cytotoxic Drugs to PSMA Expressing Cells

Anti-PSMA antibodies can be conjugated to substances with high cytotoxic potential, such as drugs of the maytansinoid class. Maytansinoids exert their cytotoxic effects by interfering with the formation and stabilization of microtubules. They have 100- to 1000-fold greater cytotoxic potential than conventional chemotherapeutic agents (such as doxorubicin, methotrexate, and Vinca alkaloids) (Chari, R. V. J. et al. (1992) Cancer Res. 52: 127).

Both murine and deimmunized J591 antibodies have been conjugated to the maytansinoid, DM1, via a hindered disulfide bond. This bond is cleaved intracellularly allowing release of the drug. One or more lysine residues in the constant regions of the antibodies were conjugated to a linker containing a pyridyldithio group, which was, in turn, coupled to a maytansinoid toxin. A ratio of 3 to 4 moles of maytansinoid per mole of IgG is preferred.

The process for the DM1-linked J591 antibodies starts by reacting J591 with a linker that contains both a pyridyldithio group and a N-hydroxysuccinimide leaving group. In this case, the linker was N-succinimidyl 4-(2-pyridyldithio)propionate (or SPP), although other linkers can be used. The products of the reaction include modified J591 antibodies that contain one or more linker groups (4-(2-pyridyldithio)propionone) attached to surface exposed lysine groups, with the linker groups retaining the pyridyldithio reactive groups, and N-hydroxysuccinimide leaving groups. The J591 antibodies are then separated from the reaction mixture and N-hydroxysuccinimide by gel filtration, e.g., using sephadex G25. The modified J591 antibodies are reacted with DM1, which contains a thiol group that reacts with the pyridyldithio groups now present on the surface of the modified antibody, thereby producing J591-DM1 immunoconjugates and thiopyridine. The J591-DM1 immunoconjugate is isolated from the reaction mixture and thiopyridine by size exclusion chromatography, e.g., using a sephacryl S300 column. Methods for preparing maytansinoid conjugates are described in U.S. Pat. Nos. 5,208,020; 5,475,092; 5,585,499; 5,846,545; and 6,333,410, the contents of which are incorporated by reference.

### Example 4

### Conjugation of deJ591 to the Maytansinoid Cytotoxin DM1

This example describes a process for the production of the deJ591-DM1 immunoconjugate. The process is based on standard methods known in the art and can therefore be generalized to other antibodies, including other antibodies of the invention such as deJ415.

The methods of conjugation are based on several small scale experiments, including one experiment performed using 5 g of deJ591 starting material (Lot 1552-60S) and three experiments performed using between 6.7 g and 7.3 g of deJ591 starting material (Lots 1552-168, 1552-104, and 1610-036).

The steps involved in the methods of conjugation are as follows:

1) 5 g to 7.5 g of deJ591 antibody is concentrated by tangential flow filtration (10 kD NMWCO membranes) to 25-30 mg/ml and diafiltered against 5 volumes of 50 mM potassium phosphate, 2 mM EDTA, pH 6.0. The yield is typically between 98% and 100%.

2) The concentrated antibody is filtered through a 0.2μ filter, if opalescent, and then modified with N-succinimidyl 4-(2-pyridyldithio)propionate (SPP) at a concentration of 20-22 mg/ml antibody and 7 molecules of SPP per molecule of antibody. The modification is done in 50 mM potassium phosphate, 2 mM EDTA, 5% ethanol, pH 6.0, for 2.5+/−0.5 hours. The modification vessel is a 500 ml round bottom flask.

3) The modified antibody is separated from the reaction mixture of step 2) using gel filtration chromatography and a Sephadex G-25™ column. The column load represents about 25% of the column volume and the chromatography is done in 50 mM potassium phosphate, 2 mM EDTA, pH 6.0, at a flow rate of 50 cm/hr. The modified antibody elutes between 38-75% column volume. Typically the yield of this step between 95% and 100% and the SPP to antibody ration is about 5.4 to 5.9 SPP molecule/antibody.

4) At a concentration of about 10 mg/ml, the modified antibody is conjugated with DM1 (using 1.7 molecules of DM1/molecule of SPP conjugated to the antibodies) for 20+/−4 hours. Typically, the reaction time is between 16.25 and 17.7 hours and is carried out in a 1 L round bottom glass flask equipped with a magnetic stirring bar. The conjugation reaction is done in 3% DMA, 10% sucrose (100 mg sucrose/ml of reaction). At the end of the reaction the conjugated antibody is filtered through a 2.0μ filter and a spectrophotometric reading is taken.

5) The conjugated antibody is separated from unreacted DM1 by gel filtration chromatography using a Sephadex G-25™ column. The column load represents 22-23% of the column volume and the flow rate is about 50 cm/hr. The column is equilibrated and run in 20 mM succinate, 5% sucrose (50 mg/ml), pH 5.5. The antibody conjugate elutes between about 31% and 65% of column volume, and is collected from the start of the peak elution to the start of the peak trailing edge as a single fraction, followed by fractionation of the remaining peak material in 1 5×2% column volume fractions. All fractions are adjusted to 100 mg/ml of sucrose (10% sucrose) through the addition of appropriate amounts of 50% sucrose. The 2% column volume fractions are assayed by analytical sizing (TSK 3000SWL) and selected fractions (fractions 1 and 2) are pooled together with the main peak. The fractions are assayed using analytical sizing with the pooling criterion being the 24 minute peak representing <20% or the total peak area. Typically the yield of this step is between 60% and 65% with the exception of run 1552-104 where there was no sucrose present in the reaction and/or purification mixture. The eluted antibody concentration ranges from 3.8 to 4.2 mg/ml and the ration of DM1/antibody ranges from 3.6 to 3.9.

6) The antibody conjugate is then concentrated to 7-10 mg/ml using a 10 kD NMWCO tangential flow filtration membrane and diafiltered against 5 volumes of 50 mM succinate, 10% sucrose, pH 5.5 (Inlet Pressure<10 psi). Following diafiltration the antibody conjugate is adjusted to 5 mg/ml. Typical yield for this step is between 92% and 100%, with the final protein concentration being between 4.85 and 5.1 mg/ml.

7) Finally, the antibody conjugate is filtered through a 0.2 m filter and aliquoted to the specified volumes. Step yield is between 90% and 100% and the final DM1-antibody ratio is 3.5 to 3.8.

The resulting deJ591-DM1 conjugates were analyzed according to appearance, concentration, DM1/antibody ratio, endotoxin, non-specific cytotoxicity, acetone extractable DM1, analytical sizing, reduced and non-reduced SDS-PAGE, pH, bioburden, specific cytotoxicity, and IEF. Selected analytical results for lots 1552-168, 1552-104, and 1552-036 are shown in Table 15, below.

TABLE 15

| | | | | Process |
|---|---|---|---|---|
| | Amount | Concentration | | Recovery |
| Lot No. | Recovered | (mg/ml) | DM1/antibody | (%) |
| 1552-168 | 3.85 | 5.1 | 3.7 | 57.2 |
| 1552-104 | 2.75 | 4.85 | 3.5 | 37.8* |
| 1610-036 | 3.41 | 5.05 | 3.8 | 47.2 |
| Mean | 3.34 | 5.00 | 3.67 | 47.4 |
| Standard Dev. | 0.55 | 0.13 | 0.15 | 9.7 |
| % c.v. | 16.6 | 2.6 | 4.2 | 20.5 |

*Lower recovery due partly to the lack of sucrose in the conjugation reaction and the second G-25 gel filtration run and partly to the fact that the front end of the product peak was not collected due to a malfunction in the chart recorder.

### Example 5

### Radiolabeling of DOTA-deJ591 with ¹¹¹In, ⁹⁰Y, and ¹⁷⁷Lu

a) Radiolabeling with ¹¹¹In

The following radiolabeling procedure can be used for the routine preparation of ¹¹¹In-DOTA-J591 for clinical studies and stability studies. Radiolabeling is achieved by the addition of ¹¹¹In chloride and Ammonium acetate buffer (1 M) to DOTA-J591 solution (8 mg/ml, 0.3 M Ammonium acetate, pH 7). To avoid the effects of autoradiolysis on the antibody, the reaction time has been minimized. The labeled ¹¹¹In-DOTA-J591 is purified using a size exclusion column and sterile filtered using a 0.2μ Millipore membrane filter prior to administration to patients. Briefly, ammonium acetate, (10 μL for each mCi of ¹¹¹In) is added to a reaction vial containing ¹¹¹In-chloride solution. Subsequently, DOTA-J591 solution (30 μL or 0.24 mg for each mCi of ¹¹¹In) is added to the reaction vial and the mixture is gently mixed and incubated at 37° C. for 20-30 min. An aliquot of the mixture is tested to determine labeling efficiency using ITLC (SG and 5 mM DTPA, pH 5). If the binding is optimal (>70%), the reaction is stopped by the addition of 10-40 μL of 5 mM DTPA.

In order to separate or purify ¹¹¹In-DOTA-J591 from free ¹¹¹In, the reaction mixture is applied on a Biogel-P6DG column (Bio-Rad, CA), prewashed with 4×10 ml of PBS containing 1% Human Serum Albumin (meets specification for US licensed albumin; manufactured by Central Laboratory Blood Transfusion Service Swiss Red Cross, Bern, Switzerland, License No. 647). The ¹¹¹In-DOTA-J591 is eluted from the column using PBS with 1% HSA and the fractions containing the labeled antibody (typically 5-8 ml) are collected into a sterile container. Following determination of radiochemical purity using ITLC (as before), and if the labeling efficiency is >95%, the labeled complex is filtered into a sterile vial using 0.2μ Filter. The final specific activity is typically 3-5 mCi/mg of antibody.

b) Radiolabeling with ⁹⁰Y

The procedure is identical to the procedure described above for ¹¹¹In, except the incubation time is 10-15 min. Radiochemical purity of ⁹⁰Y-DOTA-J591 must be >97%.

c) Radiolabeling with ¹⁷⁷Lu

The procedure is similar to the procedure described above except for two changes. The amount of Ammonium acetate added is reduced (3-5 μL for each mCi of ¹⁷⁷Lu) and the incubation time is only 5 min. Radiochemical purity of ¹⁷⁷Lu-DOTA-J591 should be >97%.

Alternative Procedures

a) Radiolabeling with ¹¹¹In

The following radiolabeling procedure is described for ¹¹¹In, but may be used with other radiolabels such as ⁹⁰Y or ¹⁷⁷Lu. Radiolabeling is achieved by adding ¹¹¹In (in dilute HCl) to ammonium acetate buffered DOTA-deJ591. To avoid the effects of autoradiolysis on the antibody, the reaction time has been minimized and the reaction mixture purified with a size exclusion column prior to administration. Briefly, a mixture composed of 20 mL of ¹¹¹In C13 (8 mCi, 0.01 M HCl, 400 mL DOTA-deJ591 (4 mg/ml, 0.3 M NH4OAc, pH 7) is allowed to react at 37° C. for 20 minutes. The reaction mixture is then separated on a 16 mL Biogel-P6DG column (Bio-Rad, CA) equilibrated with 4×10 mL of sterile 1% HSA in PBS (HAS meets specification for US licensed albumin; manufactured by Central Laboratory Blood Transfusion Service Swiss Red Cross, Bern, Switzerland, License No. 647). Once the reaction mixture is loaded onto the column, it is washed with a further 2 mL of 1% HSA PBS, before the main ¹¹¹In-DOTA-deJ591 fraction is eluted with 5 mL of 1% HSA PBS. The purified ¹¹¹In-DOTA-deJ591 is then sterile filtered into a sterile evacuated vial. Using this method, specific activity of 7.6 mCi ¹¹¹In/mg DOTA-deJ591 is achieved.

b) Radiolabeling (¹⁷⁷Lu)

The radiolabeling of DOTA-huJ591 with ¹⁷⁷Lu is achieved by adding the radionuclide (in dilute HCl) to ammonium acetate buffered DOTA-hu-J591. To avoid the effects of autoradiolysis on the antibody the reaction time has been minimized and the reaction mixture purified with a size exclusion column prior to administration. Briefly, a mixture composed of 20 μL of ¹⁷⁷Lu (30 mCi, 0.01 M HCl, MURR), 1000 μL DOTA-hu-J591 (4 mg/ml, 0.3 M NH4OAc, pH 7) is allowed to react at 37 C for 10 minutes. The reaction mixture is then separated on a 18 mL Biogel-P6DG column (Bio-Rad, CA) equilibrated with 4×10 mL of sterile 1% HSA in PBS. Once the reaction mixture is loaded onto the column, it is washed with a further 4 mL of 1% HSA PBS, before the main ¹⁷⁷Lu-DOTA-hu-J591 fraction is eluted with 2 mL of 1% HSA PBS. The purified ¹⁷⁷Lu-DOTA-hu-J591 is then sterile filtered into a sterile evacuated vial. Using this method, specific activity of 8 mCi ¹⁷⁷Lu/mg DOTA-hu-J591 have been achieved.

### Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of treating or preventing an insulin-related disorder in a subject, the method comprising administering to the subject a therapeutically effective amount of an antibody or antigen-binding portion thereof specific for PSMA.

2. The method of claim 1, wherein the subject has a co-existing, aggravating, or precipitating condition characterized by unwanted cellular proliferation in addition to the insulin-related disorder.

3. The method of claim 1, wherein the insulin-related disorder is selected from the group consisting of obesity, hyperglycemia, hypoglycemia, hyperinsulinemia, insulin-resistance, impaired glucose tolerance (IGT), impaired fasting glucose (IFG); Type 1 diabetes mellitus, Type 2 diabetes mellitus; and gestational diabetes.

4. The method of claim 2, wherein the condition characterized by unwanted cell proliferation is selected from the group consisting of cancer and non-malignant, hyperproliferative disorders.

5. The method of claim 4, wherein the cancer is prostate cancer.

6. The method of claim 1, wherein the subject is a human.

7. The method of claim 1, wherein the antibody or antigen-binding portion thereof specific for PSMA is an anti-PSMA monospecific antibody or an antigen-binding fragment thereof.

8. The method of claim 1, wherein the insulin-related disorder is diabetes.

9. The method of claim 8, further comprising monitoring the subject for an improvement in one or more indicators of diabetes.

10. The method of claim 9, wherein the improvement in one or more indicators of diabetes is reduced insulin dependence.

11. The method of claim 9, wherein the improvement in one or more indicators of diabetes is a reduction in a symptom selected from the group consisting of diabetic neuropathy, diabetic foot disease, renal impairment, heart disease, and diabetic retinopathy.

12. The method of claim 1, wherein the antibody or antigen-binding portion thereof specific for PSMA is administered in combination with a systemic agent selected from the group consisting of systemic glucocorticoids, sulfones, aminoquinolines, cytotoxic agents, antimetabolic agents, retinoids, antihistamines, immunosuppressive drugs, immunomodulatory drugs, and thalidomide.

13. The method of claim 12, wherein the antibody or antigen-binding portion thereof specific for PSMA and the systemic agent are administered sequentially

14. The method of claim 12, wherein the antibody or antigen-binding portion thereof specific for PSMA and the systemic agent are administered simultaneously.

15. The method of claim 1, wherein the antibody or antigen-binding portion thereof specific for PSMA is administered in combination with at least one agent useful in the treatment of an insulin-related disorder.

16. The method of claim 15, wherein the at least one agent useful in the treatment of diabetes is selected from the group consisting of insulin, a sulfonylurea, a biguanide, a thiazolidinedione, and an alpha-glucosidase inhibitor.

17. The method of claim 16, wherein the sulfonylurea is selected from the group consisting of a meglitinide and a nateglinide.
